SEQUENCE LISTING (44 sequences)

SEQ ID NO: 1 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 2 - protein, 13 aa, Artificial Sequence
  Synthetic Construct
FSLSTGGVGVGWI

SEQ ID NO: 3 - protein, 20 aa, Artificial Sequence
  Synthetic Construct
LALIDWADDKYYSPSLKSRL

SEQ ID NO: 4 - protein, 15 aa, Artificial Sequence
  Synthetic Construct
ARGGSDTVIGDWFAY

SEQ ID NO: 5 - protein, 11 aa, Artificial Sequence
  Synthetic Construct
RASQSIGSYLA

SEQ ID NO: 6 - protein, 9 aa, Artificial Sequence
  Synthetic Construct
DASNLETGV

SEQ ID NO: 7 - protein, 10 aa, Artificial Sequence
  Synthetic Construct
YCQQGYYLWT

SEQ ID NO: 8 - protein, 123 aa, Artificial Sequence
  Synthetic Construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSS

SEQ ID NO: 9 - protein, 107 aa, Artificial Sequence
  Synthetic Construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 10 - protein, 450 aa, Artificial Sequence
  Synthetic Construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 213 aa, Artificial Sequence
  Synthetic Construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 12 aa, Artificial Sequence
  Synthetic Construct
YSITSGHYWAWI

SEQ ID NO: 13 - protein, 21 aa, Artificial Sequence
  Synthetic Construct
VSSISGYGSTTYYADSVKGRF

SEQ ID NO: 14 - protein, 15 aa, Artificial Sequence
  Synthetic Construct
ARGGSDAVLGDWFAY

SEQ ID NO: 15 - protein, 11 aa, Artificial Sequence
  Synthetic Construct
RASQGIGSFLA

SEQ ID NO: 16 - protein, 9 aa, Artificial Sequence
  Synthetic Construct
DASNLETGV

SEQ ID NO: 17 - protein, 10 aa, Artificial Sequence
  Synthetic Construct
YCQQGYYLWT

SEQ ID NO: 18 - protein, 123 aa, Artificial Sequence
  Synthetic Construct
EVQLVESGGGLVQPGGSLRLSCAASGYSITSGHYWAWIRQAPGKGLEWVSSISGYGSTTYYADSVKGRFTISRDNSKNTLYLQLNSLRAEDTAVYYCARGGSDAVLGDWFAYWGQGTLVTVSS

SEQ ID NO: 19 - protein, 107 aa, Artificial Sequence
  Synthetic Construct
DIQLTQSPSSLSASVGDRVTITCRASQGIGSFLAWYQQKPGKAPKLLIYDASNLETGVPSRFSGSGSGTDFTLTISSLQPEDFATYYCQQGYYLWTFGQGTKVEIKR

SEQ ID NO: 20 - protein, 450 aa, Artificial Sequence
  Synthetic Construct
EVQLVESGGGLVQPGGSLRLSCAASGYSITSGHYWAWIRQAPGKGLEWVSSISGYGSTTYYADSVKGRFTISRDNSKNTLYLQLNSLRAEDTAVYYCARGGSDAVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 213 aa, Artificial Sequence
  Synthetic Construct
DIQLTQSPSSLSASVGDRVTITCRASQGIGSFLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 13 aa, Artificial Sequence
  Synthetic Construct
FSLSTSGVGVGWI

SEQ ID NO: 23 - protein, 20 aa, Artificial Sequence
  Synthetic Construct
LALIDWDDDKYYSPSLKSRL

SEQ ID NO: 24 - protein, 15 aa, Artificial Sequence
  Synthetic Construct
ARGGSDTVLGDWFAY

SEQ ID NO: 25 - protein, 11 aa, Artificial Sequence
  Synthetic Construct
RASQSVSPYLA

SEQ ID NO: 26 - protein, 9 aa, Artificial Sequence
  Synthetic Construct
DASSLESGV

SEQ ID NO: 27 - protein, 10 aa, Artificial Sequence
  Synthetic Construct
YCQQGYSLWT

SEQ ID NO: 28 - protein, 123 aa, Artificial Sequence
  Synthetic Construct
EVQLVESGGGLVQPGGSLRLSCAASGFSLSTSGVGVGWIRQAPGKGLEWLALIDWDDDKYYSPSLKSRLTISRDNSKNTLYLQLNSLRAEDTAVYYCARGGSDTVLGDWFAYWGQGTLVTVSS

SEQ ID NO: 29 - protein, 107 aa, Artificial Sequence
  Synthetic Construct
DIQLTQSPSSLSASVGDRVTITCRASQSVSPYLAWYQQKPGKAPKLLIYDASSLESGVPSRFSGSGSGTDFTLTISSLQPEDFATYYCQQGYSLWTFGQGTKVEIKR

SEQ ID NO: 30 - protein, 450 aa, Artificial Sequence
  Synthetic Construct
EVQLVESGGGLVQPGGSLRLSCAASGFSLSTSGVGVGWIRQAPGKGLEWLALIDWDDDKYYSPSLKSRLTISRDNSKNTLYLQLNSLRAEDTAVYYCARGGSDTVLGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 213 aa, Artificial Sequence
  Synthetic Construct
DIQLTQSPSSLSASVGDRVTITCRASQSVSPYLAWYQQKPGKAPKLLIYDASSLESGVPSRFSGSGSGTDFTLTISSLQPEDFATYYCQQGYSLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 11 aa, Artificial Sequence
  Synthetic Construct
  VARIANT 1: Xaa = F or Y
  VARIANT 4: Xaa = S or T
  VARIANT 5: Xaa = G, N, or S
  VARIANT 7: Xaa = A, G, or W
XTFXXYXIHWV

SEQ ID NO: 33 - protein, 12 aa, Artificial Sequence
  Synthetic Construct
  VARIANT 4: Xaa = S or T
  VARIANT 7: Xaa = H or Y
  VARIANT 8: Xaa = H or Y
  VARIANT 10: Xaa = A, D, G, N, S, or T
YSIXSGXXWXWI

SEQ ID NO: 34 - protein, 13 aa, Artificial Sequence
  Synthetic Construct
  VARIANT 6: Xaa = G or S
  VARIANT 9: Xaa = A or G
  VARIANT 11: Xaa = A, G, S, or T
FSLSTXGVXVXWI

SEQ ID NO: 35 - protein, 20 aa, Artificial Sequence
  Synthetic Construct
  VARIANT 7: Xaa = A, D, or Y
  VARIANT 8: Xaa = D or G
  VARIANT 11: Xaa = R, S, or Y
  VARIANT 14: Xaa = P or T
LALIDWXXDKXYSXSLKSRL

SEQ ID NO: 36 - protein, 20 aa, Artificial Sequence
  Synthetic Construct
  VARIANT 3: Xaa = D or E
  VARIANT 9: Xaa = N or S
  VARIANT 13: Xaa = N or S
IGXIYHSGXTYYXPSLKSRV

SEQ ID NO: 37 - protein, 21 aa, Artificial Sequence
  Synthetic Construct
  VARIANT 3: Xaa = A, G, S, V, or Y
  VARIANT 7: Xaa = A, D, S, or Y
  VARIANT 9: Xaa = D, G, or S
  VARIANT 10: Xaa = S or T
VSXISGXGXXTYYADSVKGRF

SEQ ID NO: 38 - protein, 15 aa, Artificial Sequence
  Synthetic Construct
  VARIANT 3: Xaa = E or G
  VARIANT 5: Xaa = E or S
  VARIANT 6: Xaa = D or T
  VARIANT 7: Xaa = A, T, or V
  VARIANT 9: Xaa = A, I, L, T, or V
  VARIANT 14: Xaa = A, D, or G
ARXGXXXVXGDWFXY

SEQ ID NO: 39 - protein, 11 aa, Artificial Sequence
  Synthetic Construct
  VARIANT 1: Xaa = Q or R
  VARIANT 5: Xaa = D, G, or S
  VARIANT 6: Xaa = I or V
  VARIANT 7: Xaa = G, R, S, or T
  VARIANT 8: Xaa = P, R, S, or T
  VARIANT 9: Xaa = A, D, F, S, V, or Y
  VARIANT 10: Xaa = L or V
  VARIANT 11: Xaa = A, G, or N
XASQXXXXXXX

SEQ ID NO: 40 - protein, 9 aa, Artificial Sequence
  Synthetic Construct
  VARIANT 1: Xaa = A or D
  VARIANT 4: Xaa = N, S, or T
  VARIANT 5: Xaa = L or R
  VARIANT 6: Xaa = A, E, or Q
  VARIANT 7: Xaa = S or T
  VARIANT 9: Xaa = I or V
XASXXXXGX

SEQ ID NO: 41 - protein, 10 aa, Artificial Sequence
  Synthetic Construct
  VARIANT 5: Xaa = A, G, S, or Y
  VARIANT 7: Xaa = Q, S, or Y
  VARIANT 8: Xaa = I, L, T, or Y
  VARIANT 9: Xaa = I, S, V, or W
YCQQXYXXXT

SEQ ID NO: 42 - protein, 11 aa, Artificial Sequence
  Synthetic Construct
  VARIANT 3: Xaa = E or Q
  VARIANT 5: Xaa = P, S, or Y
  VARIANT 6: Xaa = D, L, S, T, or Y
  VARIANT 7: Xaa = D, E, H, S, or T
  VARIANT 8: Xaa = D, L, T, or W
  VARIANT 10: Xaa = L, P, R, or V
YCXQXXXXPXT

SEQ ID NO: 43 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 44 - protein, 134 aa, Artificial Sequence
  Synthetic Construct
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

(19) United States
(12) Patent Application Publication
(10) Pub. No.: US 2023/0242663 A1
(43) Pub. Date: Aug. 3, 2023

(54) COMBINATION THERAPY COMPRISING ANTI-CD137 ANTIBODIES

(71) Applicant: Adagene PTE. LTD., Singapore (SG)

(72) Inventors: Peter Peizhi LUO, Lansdale, PA (US); Guizhong LIU, Suzhou, Jiangsu (CN); Aaron NGUYEN, Suzhou, Jiangsu (CN)

(73) Assignee: Adagene PTE. LTD., Singapore (SG)

(21) Appl. No.: 18/011,801

(22) PCT Filed: Jun. 23, 2021
(86) PCT No.: PCT/US2021/038718

§ 371 Date: Dec. 20, 2022

Related U.S. Application Data
(60) Provisional application No. 63/043,042, filed on Jun. 23, 2020.

(51) Int. Cl. C07K 16/28; A61P 35/00; C07K 14/55

(52) U.S. Cl. CPC C07K 16/2896; A61P 35/00; C07K 14/55; C07K 2317/33; C07K 2317/52; C07K 2317/56

(57) ABSTRACT

The present application provides compositions and methods for treating cancers in a subject using an anti-CD137 antibody and an agent that induces expression of CD137 on an immune cell and/or induces expression of CD137L on a cancer cell of the subject. In some embodiments, the agent is a cytokine such as IL-2. In some embodiments, the agent is a histone deacetylase (HDAC) inhibitor. In some embodiments, the agent is a DNA-damaging agent.

### CROSS REFERENCE TO RELATED APPLICATIONS

This application claims the priority benefit of U.S. Provisional Application No. 63/043,042, filed on Jun. 23, 2020, which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 695402000740SEQLIST.txt, date recorded: Jun. 21, 2021, size: 38 KB).

### FIELD

The present application is in the field of cancer therapeutics, and relates to compositions and methods for treating cancers using antibodies that bind to human CD137.

### BACKGROUND

CD137 (also referred to as CD137 receptor, 4-1BB, TNFRSF9, etc.) is a transmembrane protein of the Tumor Necrosis Factor Receptor Superfamily (TNFRS). Current understanding of CD137 indicates that its expression is generally activation dependent and is present in a broad subset of immune cells including activated NK and NKT cells, regulatory T cells, dendritic cells (DC), stimulated mast cells, differentiating myeloid cells, monocytes, neutrophils, and eosinophils (Wang, 2009, Immunological Reviews 229: 192-215). CD137 expression has also been demonstrated on tumor vasculature (Broll, 2001, Amer. J. Clin. Pathol. 115(4):543-549; Seaman, 2007, Cancer Cell 11: 539-554) and at sites of inflamed or atherosclerotic endothelium (Drenkard, 2007 FASEB J. 21: 456-463; Olofsson, 2008, Circulation 117: 1292-1301). The ligand that stimulates CD137, i.e., CD137 Ligand (CD137L), is expressed on activated antigen-presenting cells (APCs), myeloid progenitor cells, and hematopoietic stem cells.

Numerous studies of murine and human T cells indicate that CD137 promotes enhanced cellular proliferation, survival, and cytokine production (Croft, 2009, Nat Rev Immunol 9:271-285). Studies have indicated that some CD137 agonist monoclonal antibodies (mAbs) increase costimulatory molecule expression and markedly enhance cytolytic T lymphocyte responses, resulting in anti-tumor efficacy in various models. CD137 agonist mAbs have demonstrated efficacy in prophylactic and therapeutic settings. Further, CD137 monotherapy and combination therapy tumor models have established durable anti-tumor protective T cell memory responses (Lynch, 2008, Immunol Rev. 22: 277-286). CD137 agonists also have been shown to inhibit autoimmune reactions in a variety of art-recognized autoimmunity models (Vinay, 2006, J Mol Med 84:726-736). This dual activity of CD137 offers the potential to provide anti-tumor activity while dampening autoimmune side effects that can be associated with immunotherapy approaches that break immune tolerance.

### BRIEF SUMMARY

The present application provides methods for treating cancers in a subject using an anti-CD137 antibody and an agent that induces expression of CD137 on an immune cell and/or induces expression of CD137L on a cancer cell of the subject.

The present invention in one aspect provides a method of treating a cancer in a subject (e.g., a human subject), comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of an agent that induces expression of CD137 on an immune cell and/or induces expression of CD137L on a cancer cell of the subject. In some embodiments, the agent induces expression of CD137 on an immune cell of the subject. In some embodiments, the immune cell is selected from the group consisting of CD8+ T cells, regulatory T (Treg) cells, natural killer (NK) cells, and NK-T cells. In some embodiments, the agent induces expression of CD137L on a cancer cell of the subject.

In some embodiments, there is provided a method of treating a cancer in a subject (e.g., a human subject), comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of a cytokine that induces expression of CD137 on an immune cell of the subject. In some embodiments, the cytokine is selected from the group consisting of IL-2, IL-12, IL-10 and INFγ. In some embodiments, the cytokine induces expression of CD137L on a cancer cell of the subject.

In some embodiments, there is provided a method of treating a cancer in a subject (e.g., a human subject), comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of IL-2. In some embodiments, the IL-2 is a wildtype IL-2, a chemically modified IL-2 variant (e.g., a PEGylated IL-2), or an IL-2 analog. In some embodiments, the IL-2 is aldesleukin. In some embodiments, the IL-2 is a polyethylene glycol (PEG) modified IL-2, such as bempegaldesleukin. In some embodiments, the IL-2 is administered at a dose of no more than about 2.8×10⁶ IU/m² (e.g., about 7.2×10⁴ IU/kg or about 2.8×10⁶ IU/m²). In some embodiments, the IL-2 is administered twice or three times daily. In some embodiments, the IL-2 is administered no more than once every three days. In some embodiments, the IL-2 is administered at a dose of no more than about 1.4×10⁷ IU/m² (e.g., 7.2×10³ IU/kg or about 1.4×10⁷ IU/m²).

In some embodiments, there is provided a method of treating a cancer in a subject (e.g., a human subject), comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of a histone deacetylase (HDAC) inhibitor that induces expression of CD137 on an immune cell of the subject. In some embodiments, the HDAC inhibitor is selected from the group consisting of belinostat, vorinostat, romidepsin, and chidamide. In some embodiments, the HDAC inhibitor is belinostat. In some embodiments, the HDAC inhibitor induces expression of CD137L on a cancer cell of the subject.

In some embodiments, there is provided a method of treating a cancer in a subject (e.g., a human subject), comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of a DNA-damaging agent that induces expression of CD137 on an immune cell of the subject. In some embodiments, the DNA-damaging agent is a DNA chelator, such as mitomycin, bleomycin, or doxorubicin. In some embodiments, the DNA-damaging agent is an alkylating agent, such as bendamustine. In some embodiments, the DNA-damaging agent induces expression of CD137L on a cancer cell of the subject.

In some embodiments according to any one of the methods described above, the agent (including cytokine e.g., IL-2, HDAC inhibitor, and DNA-damaging agent) is administered intravenously. In some embodiments, the agent (including cytokine e.g., IL-2, HDAC inhibitor, and DNA-damaging agent) is administered prior to administration of the anti-CD137 antibody. In some embodiments, the agent (including cytokine e.g., IL-2, HDAC inhibitor, and DNA-damaging agent) and the anti-CD137 antibody are administered simultaneously.

In some embodiments according to any one of the methods described above, the method further comprises administering an effective amount of an anti-CD20 antibody. In some embodiments, the anti-CD20 antibody is rituximab.

In some embodiments according to any one of the methods described above, the method further comprises administering an effective amount of an immune checkpoint inhibitor. In some embodiments, the immune checkpoint inhibitor is an anti-PD-1 antibody, such as 2E5.

In some embodiments according to any one of the methods described above, the cancer is a liquid cancer. In some embodiments, the cancer is non-Hodgkin's lymphoma. In some embodiments, the cancer is T-cell lymphoma. In some embodiments, the cancer is B-cell lymphoma. In some embodiments, the cancer is multiple myeloma.

In some embodiments according to any one of the methods described above, the cancer is a solid cancer. In some embodiments, the cancer is selected from the group consisting of breast cancer, ovarian cancer, colorectal cancer, gastric cancer, melanoma, liver cancer, lung cancer, thyroid cancer, kidney cancer, brain cancer, cervical cancer, bladder cancer, and esophageal cancer. In some embodiments, the cancer is lung cancer. In some embodiments, the cancer is melanoma.

In some embodiments according to any one of the methods described above, the cancer is in adjuvant setting. In some embodiments, the cancer is in neoadjuvant setting.

In some embodiments according to any one of the methods described above, the anti-CD137 antibody is administered at a dose of no more than 500 mg, e.g., about 125 mg to about 500 mg. In some embodiments, the anti-CD137 antibody is administered at a dose of no more than about 10 mg/kg, e.g., about 2.5 mg/kg to about 10 mg/kg. In some embodiments, the anti-CD137 antibody is administered intravenously. In some embodiments, the anti-CD137 antibody is administered about once every three weeks.

In some embodiments according to any one of the methods described above, the cancer is advanced-stage cancer. In some embodiments, the cancer is metastatic cancer.

In some embodiments according to any one of the methods described above, the anti-CD137 antibody is cross-reactive with a CD137 polypeptide from at least one non-human species selected from the group consisting of cynomolgus monkey, mouse, rat and dog. In some embodiments, the anti-CD137 antibody binds to amino acid residues 51, 63-67, 69-73, 83, 89, 92, 98-104 and 112-114 of SEQ ID NO: 1.

In some embodiments according to any one of the methods described above, the anti-CD137 antibody comprises a heavy chain variable region (VH) and a light chain variable region (VL), wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4; and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO. 7. In some embodiments, the VH comprises the amino acid sequence of SEQ ID NO: 8, and/or the VL comprises the amino acid sequence of SEQ ID NO: 9. In some embodiments, the antibody comprises a heavy chain and a light chain, and wherein the heavy chain comprises the amino acid sequence of SEQ ID NO: 10, and/or the light chain comprises the amino acid sequence of SEQ ID NO: 11.

In some embodiments according to any one of the methods described above, the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14; and wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 17. In some embodiments, the VH comprises the amino acid sequence of SEQ ID NO: 18, and/or the VL comprises the amino acid sequence of SEQ ID NO: 19. In some embodiments, the antibody comprises a heavy chain and alight chain, wherein the heavy chain comprises the amino acid sequence of SEQ ID NO: 20, and/or the light chain comprises the amino acid sequence of SEQ ID NO: 21.

In some embodiments according to any one of the methods described above, the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24; and wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27. In some embodiments, the VH comprises the amino acid sequence of SEQ ID NO: 28, and/or the VL comprises the amino acid sequence of SEQ ID NO: 29. In some embodiments, the antibody comprises a heavy chain and a light chain, wherein the heavy chain comprises the amino acid sequence of SEQ ID NO: 30, and/or the light chain comprises the amino acid sequence of SEQ ID NO: 31.

In some embodiments according to any one of the methods described above, the anti-CD137 antibody comprises a human IgG1 Fc region. In some embodiments, the anti-CD137 antibody comprises a human IgG4 Fc region. In some embodiments, the human IgG4 Fc region comprises an S241P mutation, wherein numbering is according to Kabat.

Also provided are compositions, kits, and articles of manufacture for use in any one of the methods described herein.

It is to be understood that one, some, or all of the properties of the various embodiments described above and herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIGS. 1A-1B show CD137 levels on the surface of sorted peripheral blood mononuclear cells (PBMCs) treated with recombinant human IL-2. In each of FIG. 1A and FIG. 1B, the type of sorted PBMCs and the concentration of IL-2 in IU/ml are indicated on the x-axis, and the percentage of cells that expressed CD137 is indicated on the y-axis. Two panels of markers were used to sort the PBMCs into NK, NKT, CD8+, CD4+ and Treg cells, as shown in Table 1. FIG. 1A shows CD137 levels of cells sorted using Panel 1. FIG. 1B shows CD137 levels of cells sorted using Panel 2.

FIGS. 2A-2D show the effect of treatment with anti-CD137 antibody (ADG106) and/or a continuous high dose of IL-2 on reduction of tumor volumes in a mouse model of Lewis lung cancer. In each of FIGS. 2A-2D, the number of days post inoculation is shown on the x-axis, and the tumor volume in mm³ is shown on the y-axis. FIG. 2A shows tumor volume in individual mice treated with vehicle over time. FIG. 2B shows tumor volume in individual mice treated with anti-CD137 antibody ADG106 over time. 5 mg/kg of ADG106 was administered two times a week for 4 doses. FIG. 2C shows tumor volume in individual mice treated with IL-2 over time. 1.4×10⁷ IU/m² IL-2 was administered twice a day for 27 doses. FIG. 2D shows tumor volume in individual mice treated with ADG106 and IL-2 over time. 5 mg/kg of ADG106 was administered two times a week for 4 doses, and 1.4×10⁷ IU/m² IL-2 was administered twice a day for 13 doses.

FIGS. 3A-3F show the effect of treatment with anti-CD137 antibody (ADG106) and/or IL-2 at a low frequency high dose or a continuous low dose on reduction of tumor volume in a mouse model of Lewis lung cancer. In each of FIGS. 3A-3F, the number of days post inoculation is shown on the x-axis, and the volume of the tumor in mm³ is shown on the y-axis. FIG. 3A shows tumor volume in individual mice treated with vehicle over time. FIG. 3B shows tumor volume in individual mice treated with anti-CD137 antibody ADG106 over time. 2.5 mg/kg of ADG106 was administered two times a week for 4 doses. FIG. 3C shows tumor volume in individual mice treated with a high dose of IL-2 over time. 1.410⁷ IU/m² IL-2 was administered twice a day, every 3 days for 4 doses. FIG. 3D shows tumor volume in individual mice treated with a low dose of IL-2 over time. 2.8×10⁶ IU/m² of IL-2 was administered twice a day for 5 consecutive days, for a total of 10 doses. FIG. 3E shows tumor volume in individual mice treated with anti-CD137 antibody ADG106 and a high dose of IL-2 over time. 1.4×10⁷ IU/m² IL-2 was administered twice a day every 3 days for 4 doses, and 2.5 mg/kg of ADG106 was administered two times a week for 4 doses. FIG. 3F shows tumor volume in individual mice treated with anti-CD137 antibody ADG106 and a low dose of IL-2 over time. 2.8×10⁶ IU/m² of IL-2 was administered twice a day for 5 consecutive days, for a total of 10 doses, and 2.5 mg/kg of ADG106 was administered two times a week for 4 doses.

FIGS. 4A-4E show the effect of treatment with anti-CD137 antibody (ADG106) and/or Bendamustine on reduction of tumor volume in a mouse model of A20 B-cell lymphoma model. The number of days post inoculation is shown on the x-axis, and the volume of the tumor in mm³ is shown on the y-axis. FIG. 4A shows tumor growth curves of different treatment groups. Data points represent group mean, and error bars represent standard error of mean (SEM). FIG. 4B shows tumor volume in individual mice treated with vehicle over time. FIG. 4C shows tumor volume in individual mice treated with anti-CD137 antibody ADG106 over time. 2.5 mg/kg of ADG106 was administered two times a week for 4 doses. FIG. 4D shows tumor volume in individual mice treated with Bendamustine over time. 12.5 mg/kg of Bendamustine was administered once daily for 4 doses. FIG. 4E shows tumor volume in individual mice treated with ADG106 in combination of Bendamustine over time. 2.5 mg/kg of ADG106 was administered two times a week for 4 doses, 12.5 mg/kg of Bendamustine was administered once daily for 4 doses.

FIGS. 5A-5E show the effects of treatment with different dose of romidepsin, bortezomib, chidamide, belinostat, and vincristine on CD137L protein expression levels on HUT78 cutaneous T cell lymphoma (CTCL) cells surface. FIG. 5A shows the effects of treatment with romidepsin on CD137L protein expression levels on HUT78 CTCL cells surface. FIG. 5B shows the effects of treatment with bortezomib on CD137L protein expression levels on HUT78 CTCL cells surface. FIG. 5C shows the effects of treatment with chidamide on CD137L protein expression levels on HUT78 CTCL cells surface. FIG. 5D shows the effects of treatment with belinostat on CD137L protein expression levels on HUT78 CTCL cells surface. FIG. 5E shows the effects of treatment with vincristine on CD137L protein expression levels on HUT78 CTCL cells surface. FIG. 5F shows the effects of treatment with romidepsin on CD137L protein expression levels on HUT78 CTCL cells surface. FIG. 5G shows the effects of treatment with bortezomib on CD137L protein expression levels on HUT78 CTCL cells surface. FIG. 5H shows the effects of treatment with chidamide on CD137L protein expression levels on HUT78 CTCL cells surface. Cells in FIGS. 5A-5E were stained with PE-conjugated Isotype Control (Biolegend catalog #400112) or anti-human-CD137L (Biolegend catalog #311504) antibodies; cells in FIGS. 5F-5H were stained with PE-Cy7-conjugated Isotype Control (Thermofisher catalog #25-4714-80) and anti-human-CD137L (Thermofisher catalog #25-5906-42) antibodies.

FIGS. 6A-6B show the effects of treatment with romidepsin and bortezomib, on CD137L protein expression levels on HUT78 CTCL cells surface at different time points. FIG. 6A shows the effects of treatment with 0.003 μM romidepsin on CD137L protein expression levels on HUT78 CTCL cells surface at different time points. FIG. 6B shows the effects of treatment with 0.01 μM bortezomib on CD137L protein expression levels on HUT78 CTCL cells surface at different time points.

FIGS. 7A-7C show the effects of treatment with different dose of romidepsin on mRNA expression in HUT102, HUT78, and SU-DHL1 human T cell lymphoma (TCL) cells. FIG. 7A shows the effects of treatment with romidepsin on mRNA expression in HUT102 human TCL cells. FIG. 7B shows the effects of treatment with romidepsin on mRNA expression in HUT78 human TCL cells. FIG. 7C shows the effects of treatment with romidepsin on mRNA expression in SU-DHL1 human TCL cells. # indicates genes with low basal expression.

FIGS. 8A-8C show the effects of treatment with different dose of belinostat on mRNA expression in HUT102, HUT78, and SU-DHL1 human TCL cells. FIG. 8A shows the effects of treatment with belinostat on mRNA expression in HUT102 human TCL cells. FIG. 8B shows the effects of treatment with belinostat on mRNA expression in HUT78 human TCL cells. FIG. 8C shows the effects of treatment with belinostat on mRNA expression in SU-DHL1 human TCL cells. # indicates genes with low basal expression.

FIGS. 9A-9C show the effects of treatment with different dose of bortezomib on mRNA expression in HUT102, HUT78, and SU-DHL1 human TCL cells. FIG. 9A shows the effects of treatment with bortezomib on mRNA expression in HUT102 human TCL cells. FIG. 9B shows the effects of treatment with bortezomib on mRNA expression in HUT78 human TCL cells. FIG. 9C shows the effects of treatment with bortezomib on mRNA expression in SU-DHL1 human TCL cells. # indicates genes with low basal expression.

FIGS. 10A-10C show the effects of treatment with different dose of vincristine on mRNA expression in HUT102, HUT78, and SU-DHL1 human TCL cells. FIG. 10A shows the effects of treatment with vincristine on mRNA expression in HUT102 human TCL cells. FIG. 10B shows the effects of treatment with vincristine on mRNA expression in HUT78 human TCL cells. FIG. 10C shows the effects of treatment with vincristine on mRNA expression in SU-DHL1 human TCL cells. # indicates genes with low basal expression.

FIGS. 11A-11C show the effects of treatment with different dose of romidepsin, bortezomib, and chidamide on viability of HUT78 human TCL cells. FIG. 11A shows the effects of treatment with different dose of romidepsin on viability of HUT78 human TCL cells. FIG. 11B shows the effects of treatment with different dose of bortezomib on viability of HUT78 human TCL cells. FIG. 11C shows the effects of treatment with different dose of chidamide on viability of HUT78 human TCL cells.

FIGS. 12A-12C show the effects of treatment with different dose of romidepsin, bortezomib, and chidamide on viability of purified human T cells. FIG. 12A shows the effects of treatment with different dose of romidepsin on viability of purified human T cells. FIG. 12B shows the effects of treatment with different dose of bortezomib on viability of purified human T cells. FIG. 12C shows the effects of treatment with different dose of chidamide on viability of purified human T cells.

FIGS. 13A-13I show the effects of treatment with anti-CD137 antibody ADG106, anti-PD1 antibody 2E5, IL-2, ADG106 in combination with IL-2, 2E5 in combination with IL-2, ADG106 in combination with 2E5, and ADG106 in combination with both IL-2 and 2E5 on a B16F10 mouse model. FIG. 13A shows a comparison of the average tumor volume over time among the various treatment groups. FIG. 13B shows individual response in the ADG106 monotherapy group. FIG. 13C shows individual response in the vehicle (control) group. FIG. 13D shows individual response in the IL-2 monotherapy group. FIG. 13E shows individual response in the ADG106+IL-2 combination therapy group. FIG. 13F shows individual response in the 2E5 monotherapy group. FIG. 13G shows individual response in the ADG106+2E5 combination therapy group. FIG. 13H shows individual response in the 2E5+IL-2 combination therapy group. FIG. 13I shows individual response in the ADG106+IL-2+2E5 combination therapy group.

### DETAILED DESCRIPTION

The present application provides methods of treating cancers in a subject using an anti-CD137 antibody and an agent such as a cytokine (e.g., IL-2) or a histone deacetylase (HDAC) inhibitor that induces expression of CD137 on an immune cell and/or induces expression of CD137L on a cancer cell in the subject. The methods described herein are based at least in part on the inventors' discovery that IL-2 induces expression of CD137 on T cells, NK cells and NK-T cells, which may contribute to the synergistic effects of IL-2 and an anti-CD137 antibody in a combination therapy for treating cancer. Furthermore, although combination of an anti-CD137 antibody with a continuous high dose of IL-2 led to significant toxicity in an in vivo mouse model of lung cancer, combination of an anti-CD137 antibody with IL-2 at a low-frequency high dose or at a continuous low dose showed synergistic anti-tumor effects without incurring toxicity.

Accordingly, the present application in one aspect provides a method of treating a cancer in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1, and (b) an agent (e.g., IL-2) that induces expression of CD137 on an immune cell and/or induces expression of CD137L on a cancer cell of the subject.

### I. Definitions

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, antibody engineering, immunotherapy, cell and tissue culture, molecular biology, immunology, microbiology, genetics and protein and nucleic acid chemistry described herein are those well-known and commonly used in the art.

The terms “CD137” and “CD137 receptor” are used interchangeably in the present application, and include the human CD137 receptor, as well as variants, isoforms, and species homologs thereof. Accordingly, a binding molecule, as defined and disclosed herein, may also bind CD137 from species other than human. In other cases, a binding molecule may be completely specific for the human CD137 and may not exhibit species or other types of cross-reactivity.

The term “CD137 antibody” refers to an antibody, as defined herein, capable of binding to human CD137 receptor.

The term “antibody” is used herein in the broadest sense and specifically covers monoclonal antibodies (including full length monoclonal antibodies), polyclonal antibodies, multispecific antibodies (e.g., bispecific antibodies), and antibody fragments (e.g., a single-chain variable fragment or scFv) so long as they exhibit the desired biological activity.

The term “antibody” is an art-recognized term and may refer to an antigen-binding protein (i.e., immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as VH) followed by a constant region. The heavy chain constant region is comprised of three domains, CH1, CH2 and CH3. Each light chain has, at the N-terminus, a variable region (abbreviated herein as VL) followed by a constant region at its other end. The light chain constant region is comprised of one domain, CL. The VL is aligned with the VH and the CL is aligned with the first constant domain of the heavy chain (CH1). The pairing of a VH and VL together forms a single antigen-binding site. An IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain.

The VH and VL regions can be further subdivided into regions of hypervariability, termed hyper-variable regions (HVR) based on the structural and sequence analysis. HVRs are interspersed with regions that are more conserved, termed framework regions (FW). For comparison, the Kabat CDR definition by Yvonne Chen, et al. (Selection and Analysis of an Optimized Anti-VEGF Antibody: Crystal Structure of an Affinity-matured Fab in Complex with Antigen, J. Mol. Biol. (1999) 293, 865-881) is listed below. Each VH and VL is composed of three HVRs and four FWs, arranged from amino-terminus to carboxy-terminus in the following order FW1, HVR1, FW2, HVR2, FW3, HVR3. FW4. Throughout the present disclosure, the three HVRs of the heavy chain are referred to as HVR_H1, HVR_H2, and HVR_H3. Similarly, the three HVRs of the light chain are referred to as HVR_L1, HVR_L2, and HVR_L3.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616(1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs as defined by each of the above-cited references, are set forth below in Table A as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

TABLE A
CDR DEFINITIONS

| | Kabat¹ | Chothia² | MacCallum³ | IMGT⁴ | AHo⁵ |
|---|---|---|---|---|---|
| VH CDR1 | 31-35 | 26-32 | 30-35 | 27-38 | 25-40 |
| VH CDR2 | 50-65 | 53-55 | 47-58 | 56-65 | 58-77 |
| VH CDR3 | 95-102 | 96-101 | 93-101 | 105-117 | 109-137 |
| VL CDR1 | 24-34 | 26-32 | 30-36 | 27-38 | 25-40 |
| VL CDR2 | 50-56 | 50-52 | 46-55 | 56-65 | 58-77 |
| VL CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Within light and heavy chains, the variable and constant regions are joined by a “J” region of about 12 or more amino acids, with the heavy chain also including a “D” region of about 10 or more amino acids. See generally, Fundamental Immunology Ch. 7 (Paul, W., ed., 2^{nd}ed. Raven Press, N.Y. (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. There are five classes of antibodies: IgA, IgD, IgE, IgG, and IgM, having heavy chains designated α (alpha), δ (delta), ε (epsilon), γ (gamma), and μ (mu), respectively. The IgG class of antibody can be further classified into four subclasses IgG1, IgG2, IgG3, and IgG4 by the gamma heavy chains, Y1-Y4, respectively.

“Fc region” as used herein refers to the polypeptide comprising the constant region of an antibody heavy chain excluding the first constant region immunoglobulin domain. For IgG, the Fc region may comprise immunoglobulin domains CH2 and CH3 and the hinge between CH1 and CH2.

The term “antibody derivative” or “derivative” of an antibody refers to a molecule that is capable of binding to the same antigen (e.g., CD137) that the antibody binds to and comprises an amino acid sequence of the antibody linked to an additional molecular entity. The amino acid sequence of the antibody that is contained in the antibody derivative may be a full-length heavy chain, a full-length light chain, any portion or portions of a full-length heavy chain, any portion or portions of the full-length light chain of the antibody, any other fragment(s) of an antibody, or the complete antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional molecular entity by chemical coupling, genetic fusion, noncovalent association, or otherwise. The term “antibody derivative” also encompasses chimeric antibodies, humanized antibodies, and molecules that are derived from modifications of the amino acid sequences of an antibody (e.g., a CD137 antibody), such as conservation amino acid substitutions, additions, and insertions.

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see, e.g. Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucleic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

The term “antigen-binding fragment” or “antigen binding portion” of an antibody refers to one or more portions of an antibody that retain the ability to bind to the antigen that the antibody bonds to (e.g., CD137). Examples of “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546 (1989)), which consists of a VH domain; and (vi) an isolated complementarity determining region (CDR).

The term “binding molecule” encompasses (1) antibody, (2) antigen-binding fragment of an antibody, and (3) derivative of an antibody, each as defined herein.

The term “binding CD137,” “binds CD137,” “binding to CD137,” or “binds to CD137” refers to the binding of a binding molecule, as defined herein, to the human CD137 in an in vitro assay, such as a Biacore assay, with an affinity (K_{D}) of 100 nM or less.

The term “specifically binds” or “specifically binds to,” in reference to the interaction of a binding molecule, as defined herein, (e.g., an antibody) with its binding partner (e.g., an antigen), refers to the ability of the binding molecule to discriminate between an antigen of interest from an animal species and the antigen orthologue from a different animal species under a given set of conditions. A CD137 binding molecule is said to specifically bind to human CD137 if it binds to human CD137 at an EC50 that is below 50 percent of the EC50 at which it binds CD137 of rat or mouse as determined in an in vitro assay. Binding specificity of an antibody can be determined using methods known in the art. Examples of such methods include FACS using PHA stimulated primary cells, Western blots, ELISA-, RIA-, ECL-, IRMA-tests and peptide scans.

The term “compete for binding” refers to the interaction of two antibodies in their binding to a binding target. A first antibody competes for binding with a second antibody if binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not, be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s).

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants. See, e.g., Epitope Mapping Protocols in Methods in Molecular Biology, Vol. 66, G. E. Morris, Ed. (1996). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

The term “human antibody” refers to an antibody in which the entire amino acid sequences of the light chains and heavy chains are from the human immunoglobulin genes. A human antibody may contain murine carbohydrate chains if produced in a mouse, in a mouse cell or in a hybridoma derived from a mouse cell. Human antibodies may be prepared in a variety of ways known in the art.

The term “humanized antibody” refers to a chimeric antibody that contains amino acid residues derived from human antibody sequences. A humanized antibody may contain some or all of the CDRs or HVRs from a non-human animal or synthetic antibody while the framework and constant regions of the antibody contain amino acid residues derived from human antibody sequences.

The term “chimeric antibody” refers to an antibody that comprises amino acid sequences derived from different animal species, such as those having a variable region derived from a human antibody and a murine immunoglobulin constant region.

The term “isolated antibody” or “isolated binding molecule” refers to an antibody or a binding molecule, as defined herein, that: (1) is not associated with naturally associated components that accompany it in its native state; (2) is free of other proteins from the same species; (3) is expressed by a cell from a different species; or (4) does not occur in nature. Examples of isolated antibodies include a CD137 antibody that has been affinity purified using CD137, a CD137 antibody that has been generated by hybridomas or other cell line in vitro, and a CD137 antibody derived from a transgenic animal.

The term “isolated nucleic acid” refers to a nucleic acid molecule of genomic, cDNA, or synthetic origin, or a combination thereof, which is separated from other nucleic acid molecules present in the natural source of the nucleic acid. For example, with regard to genomic DNA, the term “isolated” includes nucleic acid molecules, which are separated from the chromosome with which the genomic DNA is naturally associated. Preferably, an “isolated” nucleic acid is free of sequences, which naturally flank the nucleic acid (i.e., sequences located at the 5′ and 3′ ends of the nucleic acid of interest.

An “individual” or a “subject” is a mammal, more preferably a human. Mammals also include, but are not limited to, farm animals, sport animals, pets (such as cats, dogs, and horses), primates, mice and rats.

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “prevent” or “preventing,” with reference to a certain disease condition in a mammal, refers to preventing or delaying the onset of the disease, or preventing the manifestation of clinical or subclinical symptoms thereof.

As used herein, an “effective amount” refers to an amount of an agent or drug effective to treat a disease or disorder in a subject. In the case of cancer, the effective amount of the agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“Adjuvant setting” refers to a clinical setting in which an individual has had a history of cancer, and generally (but not necessarily) been responsive to therapy, which includes, but is not limited to, surgery (e.g., surgery resection), radiotherapy, and chemotherapy. Treatment or administration in the “adjuvant setting” refers to a subsequent mode of treatment.

“Neoadjuvant setting” refers to a clinical setting in which the method is carried out before the primary/definitive therapy.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

An “adverse event” or “AE” as used herein refers to any untoward medical occurrence in an individual receiving a marketed pharmaceutical product or in an individual who is participating on a clinical trial who is receiving an investigational or non-investigational pharmaceutical agent. The AE does not necessarily have a causal relationship with the individual's treatment. Therefore, an AE can be any unfavorable and unintended sign, symptom, or disease temporally associated with the use of a medicinal product, whether or not considered to be related to the medicinal product. An AE includes, but is not limited to: an exacerbation of a pre-existing illness; an increase in frequency or intensity of a pre-existing episodic event or condition; a condition detected or diagnosed after study drug administration even though it may have been present prior to the start of the study; and continuously persistent disease or symptoms that were present at baseline and worsen following the start of the study. An AE generally does not include: medical or surgical procedures (e.g., surgery, endoscopy, tooth extraction, or transfusion); however, the condition that leads to the procedure is an adverse event; pre-existing diseases, conditions, or laboratory abnormalities present or detected at the start of the study that do not worsen; hospitalizations or procedures that are done for elective purposes not related to an untoward medical occurrence (e.g., hospitalizations for cosmetic or elective surgery or social/convenience admissions); the disease being studied or signs/symptoms associated with the disease unless more severe than expected for the individual's condition; and overdose of study drug without any clinical signs or symptoms.

A “serious adverse event” or (SAE) as used herein refers to any untoward medical occurrence at any dose including, but not limited to, that: a) is fatal; b) is life-threatening (defined as an immediate risk of death from the event as it occurred); c) results in persistent or significant disability or incapacity; d) requires in-patient hospitalization or prolongs an existing hospitalization (exception: Hospitalization for elective treatment of a pre-existing condition that did not worsen during the study is not considered an adverse event. Complications that occur during hospitalization are AEs and if a complication prolongs hospitalization, then the event is serious); e) is a congenital anomaly/birth defect in the offspring of an individual who received medication; or f) conditions not included in the above definitions that may jeopardize the individual or may require intervention to prevent one of the outcomes listed above unless clearly related to the individual's underlying disease. “Lack of efficacy” (progressive disease) is not considered an AE or SAE. The signs and symptoms or clinical sequelae resulting from lack of efficacy should be reported if they fulfill the AE or SAE definitions.

The following definitions may be used to evaluate response based on target lesions: “complete response” or “CR” refers to disappearance of all target lesions; “partial response” or “PR” refers to at least a 30% decrease in the sum of the longest diameters (SLD) of target lesions, taking as reference the baseline SLD; “stable disease” or “SD” refers to neither sufficient shrinkage of target lesions to qualify for PR, nor sufficient increase to qualify for PD, taking as reference the nadir SLD since the treatment started; and “progressive disease” or “PD” refers to at least a 20% increase in the SLD of target lesions, taking as reference the nadir SLD recorded since the treatment started, or, the presence of one or more new lesions.

The following definitions of response assessments may be used to evaluate a non-target lesion: “complete response” or “CR” refers to disappearance of all non-target lesions; “stable disease” or “SD” refers to the persistence of one or more non-target lesions not qualifying for CR or PD; and “progressive disease” or “PD” refers to the “unequivocal progression” of existing non-target lesion(s) or appearance of one or more new lesion(s) is considered progressive disease (if PD for the individual is to be assessed for a time point based solely on the progression of non-target lesion(s), then additional criteria are required to be fulfilled.

“Progression free survival” (PFS) indicates the length of time during and after treatment that the cancer does not grow. Progression-free survival includes the amount of time individuals have experienced a complete response or a partial response, as well as the amount of time individuals have experienced stable disease.

The terms “polypeptide,” “protein,” and “peptide” are used interchangeably herein and may refer to polymers of two or more amino acids.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptulose, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“PEG” or “polyethylene glycol,” as used herein, is meant to encompass any water soluble poly(ethylene oxide). Unless otherwise indicated, a, “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present invention will comprise one of the two following structures: “—(CH₂CH₂O)ₙ” or “—(CH₂CH₂O)ₙ₋₁CH₂CH₂—,” depending upon whether or not the terminal oxygen(s) has been displaced, e.g., during a synthetic transformation. As stated above, for the PEG polymers, the variable (n) ranges from about 3 to 4000, and the terminal groups and architecture of the overall PEG can vary.

The methods and techniques of the present disclosure are generally performed according to methods well known in the art and as described in various general and more specific references that are cited and discussed throughout the present specification unless otherwise indicated. Such references include, e.g., Sambrook and Russell, Molecular Cloning. A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990). Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

It is understood that embodiments of the present application described herein include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A. B, and/or C” is intended to encompass each of the following embodiments: A, B, and C; A, B, or C; A or C; A or B; B or C; A and C; A and B; B and C; A (alone); B (alone); and C (alone).

### II. Methods of Treatment

The present application provides methods for treating cancers using an anti-CD137 antibody that specifically binds to an extracellular domain of human CD137 in combination with an agent (“CD137-inducing agent”) that induces expression of CD137 on immune cells and/or induces expression of CD137L on a cancer cell. Any one of the anti-CD137 antibodies in Section III “Anti-CD137 Antibodies” may be used in combination with any one of the CD137-inducing agents in the subsection “CD137-inducing agents” below for the methods described herein.

In some embodiments, there is provided a method of treating a cancer in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89,92, 95-104 and 112-116 of SEQ ID NO: 1; and (b) an effective amount of an agent that induces expression of CD137 on an immune cell (e.g., CD8+ T cells, Treg cells, NK cells and/or NK-T cells) and/or induces expression of CD137L on a cancer cell of the subject. In some embodiments, the agent induces expression of CD137 on an immune cell of the subject. In some embodiments, the agent induces expression of CD137L on a cancer cell of the subject. In some embodiments, the agent induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4, and wherein the VL comprises a HVR-L1 comprising the amino acid sequence of SEQ ID NO: 5, a HVR-L2 comprising the amino acid sequence of SEQ ID NO. 6, and a HVR-L3 comprising the amino acid sequence of SEQ ID NO: 7; and (b) an effective amount of an agent that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 8, and/or a VL comprises the amino acid sequence of SEQ ID NO: 9. In some embodiments, the agent induces expression of CD137 on an immune cell of the subject. In some embodiments, the agent induces expression of CD137L on a cancer cell of the subject. In some embodiments, the agent induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14, and wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 17; and (b) an effective amount of an agent that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 18, and/or a VL comprises the amino acid sequence of SEQ ID NO: 19. In some embodiments, the agent induces expression of CD137 on an immune cell of the subject. In some embodiments, the agent induces expression of CD137L on a cancer cell of the subject. In some embodiments, the agent induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24, and wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27; and (b) an effective amount of an agent that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 28, and/or a VL comprises the amino acid sequence of SEQ ID NO: 29. In some embodiments, the agent induces expression of CD137 on an immune cell of the subject. In some embodiments, the agent induces expression of CD137L on a cancer cell of the subject. In some embodiments, the agent induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of a cytokine that induces expression of CD137 on an immune cell an agent that induces expression of CD137 on an immune cell (e.g., CD8+ T cells, Treg cells, NK cells and/or NK-T cells) and/or induces expression of CD137L on a cancer cell of the subject. In some embodiments, the cytokine is selected from the group consisting of IL-2, IL-12, IL-10 and INFγ. In some embodiments, the cytokine induces expression of CD137 on an immune cell of the subject. In some embodiments, the cytokine induces expression of CD137L on a cancer cell of the subject. In some embodiments, the cytokine induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4, and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7; and (b) an effective amount of a cytokine that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 8, and/or a VL comprises the amino acid sequence of SEQ ID NO: 9. In some embodiments, the cytokine is selected from the group consisting of IL-2, IL-12, IL-10 and INFγ. In some embodiments, the cytokine induces expression of CD137 on an immune cell of the subject. In some embodiments, the cytokine induces expression of CD137L on a cancer cell of the subject. In some embodiments, the cytokine induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14, and wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 17; and (b) an effective amount of a cytokine that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 18, and/or a VL comprises the amino acid sequence of SEQ ID NO: 19. In some embodiments, the cytokine is selected from the group consisting of IL-2, 11-12, IL-10 and INFγ. In some embodiments, the cytokine induces expression of CD137 on an immune cell of the subject. In some embodiments, the cytokine induces expression of CD137L on a cancer cell of the subject. In some embodiments, the cytokine induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24, and wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27; and (b) an effective amount of a cytokine that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 28, and/or a VL comprises the amino acid sequence of SEQ ID NO: 29. In some embodiments, the cytokine is selected from the group consisting of IL-2, IL-12, IL-10 and INFγ. In some embodiments, the cytokine induces expression of CD137 on an immune cell of the subject. In some embodiments, the cytokine induces expression of CD137L on a cancer cell of the subject. In some embodiments, the cytokine induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of an IL-2. In some embodiments, the IL-2 is a wildtype IL-2, a chemically modified IL-2 variant, or an IL-2 analog. In some embodiments, the IL-2 is bempegaldesleukin.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4, and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7; and (b) an effective amount of an IL-2. In some embodiments, the anti-CD137 antibody comprises a VH comprising the amino acid sequence of SEQ ID NO: 8, and/or a VL comprises the amino acid sequence of SEQ ID NO: 9. In some embodiments, the IL-2 is a wildtype IL-2, a chemically modified IL-2 variant, or an IL-2 analog. In some embodiments, the IL-2 is bempegaldesleukin.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14, and wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 17; and (b) an effective amount of an IL-2. In some embodiments, the anti-CD137 antibody comprises a VH comprising the amino acid sequence of SEQ ID NO: 18, and/or a VL comprises the amino acid sequence of SEQ ID NO: 19. In some embodiments, the IL-2 is a wildtype IL-2, a chemically modified IL-2 variant, or an IL-2 analog. In some embodiments, the IL-2 is bempegaldesleukin.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24, and wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27; and (b) an effective amount of an IL-2. In some embodiments, the anti-CD137 antibody comprises a VH comprising the amino acid sequence of SEQ ID NO: 28, and/or a VL comprises the amino acid sequence of SEQ ID NO: 29. In some embodiments, the IL-2 is a wildtype IL-2, a chemically modified IL-2 variant, or an IL-2 analog. In some embodiments, the IL-2 is bempegaldesleukin.

In some embodiments, there is provided a method of treating a cancer (e.g., lung cancer or melanoma) in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of an IL-2, wherein the IL-2 is administered at a dose of no more than about 2.8×10⁶ IU/m². In some embodiments, the IL-2 is a wildtype IL-2, a chemically modified IL-2 variant, or an IL-2 analog. In some embodiments, the IL-2 is bempegaldesleukin. In some embodiments, the IL-2 is administered twice daily. In some embodiments, the IL-2 is administered at a dose of about 7.2×10⁴ IU/kg or about 2.8×10⁶ IU/m².

In some embodiments, there is provided a method of treating a cancer (e.g., lung cancer or melanoma) in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4, and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7; and (b) an effective amount of an IL-2, wherein the IL-2 is administered at a dose of no more than about 2.8×10⁶ IU/m². In some embodiments, the anti-CD137 antibody comprises a VH comprising the amino acid sequence of SEQ ID NO: 8, and/or a VL comprises the amino acid sequence of SEQ ID NO: 9. In some embodiments, the IL-2 is a wildtype IL-2, a chemically modified IL-2 variant, or an IL-2 analog. In some embodiments, the IL-2 is bempegaldesleukin. In some embodiments, the IL-2 is administered twice daily. In some embodiments, the IL-2 is administered at a dose of about 7.2×10⁴ IU/kg or about 2.8×10⁶ IU/m².

In some embodiments, there is provided a method of treating a cancer (e.g., lung cancer or melanoma) in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14, and wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 7; and (b) an effective amount of an IL-2, wherein the IL-2 is administered at a dose of no more than about 2.8×10⁶ IU/m². In some embodiments, the anti-CD137 antibody comprises a VH comprising the amino acid sequence of SEQ ID NO: 18, and/or a VL comprises the amino acid sequence of SEQ ID NO: 19. In some embodiments, the IL-2 is a wildtype IL-2, a chemically modified IL-2 variant, or an IL-2 analog. In some embodiments, the IL-2 is bempegaldesleukin. In some embodiments, the IL-2 is administered twice daily. In some embodiments, the IL-2 is administered at a dose of about 7.2×10⁴ IU/kg or about 2.8×100 IU/m².

In some embodiments, there is provided a method of treating a cancer (e.g., lung cancer or melanoma) in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24, and wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27; and (b) an effective amount of an IL-2, wherein the IL-2 is administered at a dose of no more than about 2.8×10⁶ IU/m². In some embodiments, the anti-CD137 antibody comprises a VH comprising the amino acid sequence of SEQ ID NO: 28, and/or a VL comprises the amino acid sequence of SEQ ID NO: 29. In some embodiments, the IL-2 is a wildtype IL-2, a chemically modified IL-2 variant, or an IL-2 analog. In some embodiments, the IL-2 is bempegaldesleukin. In some embodiments, the IL-2 is administered twice daily. In some embodiments, the IL-2 is administered at a dose of about 7.2×10⁴ IU/kg or about 2.8×10⁶ IU/m².

In some embodiments, there is provided a method of treating a cancer (e.g., lung cancer or melanoma) in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of an IL-2, wherein the IL-2 is administered no more than once every three days. In some embodiments, the IL-2 is a wildtype IL-2, a chemically modified IL-2 variant, or an IL-2 analog. In some embodiments, the IL-2 is bempegaldesleukin. In some embodiments, the IL-2 is administered at a dose of no more than about 1.4×10⁷ IU/m², e.g., about 7.2×10⁵ IU/kg or about 1.4×10⁷ IU/m².

In some embodiments, there is provided a method of treating a cancer (e.g., lung cancer or melanoma) in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4, and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7; and (b) an effective amount of an IL-2, wherein the IL-2 is administered no more than once every three days. In some embodiments, the anti-CD137 antibody comprises a VH comprising the amino acid sequence of SEQ ID NO: 8, and/or a VL comprises the amino acid sequence of SEQ ID NO: 9. In some embodiments, the IL-2 is a wildtype IL-2, a chemically modified IL-2 variant, or an IL-2 analog. In some embodiments, the IL-2 is bempegaldesleukin. In some embodiments, the IL-2 is administered at a dose of no more than about 1.4×10⁷ IU/m², e.g., about 7.2×10⁵ IU/kg or about 1.4×10⁷ IU/m².

In some embodiments, there is provided a method of treating a cancer (e.g., lung cancer or melanoma) in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14, and wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 17; and (b) an effective amount of an IL-2, wherein the IL-2 is administered no more than once every three days. In some embodiments, the anti-CD137 antibody comprises a VH comprising the amino acid sequence of SEQ ID NO: 18, and/or a VL comprises the amino acid sequence of SEQ ID NO: 19. In some embodiments, the IL-2 is a wildtype IL-2, a chemically modified IL-2 variant, or an IL-2 analog. In some embodiments, the IL-2 is bempegaldesleukin. In some embodiments, the IL-2 is administered at a dose of no more than about 1.4×10⁷ IU/m², e.g., about 7.2×10⁵ IU/kg or about 1.4×10⁷ IU/m².

In some embodiments, there is provided a method of treating a cancer (e.g., lung cancer or melanoma) in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24, and wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27; and (b) an effective amount of an IL-2, wherein the IL-2 is administered no more than once every three days. In some embodiments, the anti-CD137 antibody comprises a VH comprising the amino acid sequence of SEQ ID NO: 28, and/or a VL comprises the amino acid sequence of SEQ ID NO: 29. In some embodiments, the IL-2 is a wildtype IL-2, a chemically modified IL-2 variant, or an IL-2 analog. In some embodiments, the IL-2 is bempegaldesleukin. In some embodiments, the IL-2 is administered at a dose of no more than about 1.4×10⁷ IU/m², e.g., about 7.2×10⁵ IU/kg or about 1.4×10⁷ IU/m².

In some embodiments, there is provided a method of treating a cancer in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of a histone deacetylase (HDAC) inhibitor that induces expression of CD137 on an immune cell (e.g., CD8+ T cells, Treg cells, NK cells and/or NK-T cells) and/or induces expression of CD137L on a cancer cell of the subject. In some embodiments, the HDAC inhibitor is selected from the group consisting of belinostat, vorinostat, romidepsin, and chidamide. In some embodiments, the HDAC inhibitor induces expression of CD137 on an immune cell of the subject. In some embodiments, the HDAC inhibitor induces expression of CD137L on a cancer cell of the subject. In some embodiments, the HDAC inhibitor induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4, and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7; and (b) an effective amount of an HDAC inhibitor that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 8, and/or a VL comprises the amino acid sequence of SEQ ID NO: 9. In some embodiments, the HDAC inhibitor is selected from the group consisting of belinostat, vorinostat, romidepsin, and chidamide. In some embodiments, the HDAC inhibitor induces expression of CD137 on an immune cell of the subject. In some embodiments, the HDAC inhibitor induces expression of CD137L on a cancer cell of the subject. In some embodiments, the HDAC inhibitor induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14, and wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 17; and (b) an effective amount of an HDAC inhibitor that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 18, and/or a VL comprises the amino acid sequence of SEQ ID NO: 19. In some embodiments, the HDAC inhibitor is selected from the group consisting of belinostat, vorinostat, romidepsin, and chidamide. In some embodiments, the HDAC inhibitor induces expression of CD137 on an immune cell of the subject. In some embodiments, the HDAC inhibitor induces expression of CD137L on a cancer cell of the subject. In some embodiments, the HDAC inhibitor induces expression of CD137 on an immune cell of the subject and induces expression of CD137L on a cancer cell of the subject.

In some embodiments, there is provided a method of treating a cancer in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24, and wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27; and (b) an effective amount of an HDAC inhibitor that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 28, and/or a VL comprises the amino acid sequence of SEQ ID NO: 29. In some embodiments, the HDAC inhibitor is selected from the group consisting of belinostat, vorinostat, romidepsin, and chidamide. In some embodiments, the HDAC inhibitor induces expression of CD137 on an immune cell of the subject. In some embodiments, the HDAC inhibitor induces expression of CD137L on a cancer cell of the subject. In some embodiments, the HDAC inhibitor induces expression of CD137 on an immune cell of the subject and induces expression of CD137L on a cancer cell of the subject.

In some embodiments, there is provided a method of treating a cancer (e.g., B-cell lymphoma) in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of a DNA-damaging agent (e.g., bendamustine) that induces expression of CD137 on an immune cell (e.g., CD8+ T cells, Treg cells, NK cells and/or NK-T cells) and/or induces expression of CD137L on a cancer cell of the subject. In some embodiments, the DNA-damaging agent is a DNA chelator or an alkylating agent. In some embodiments, the DNA-damaging agent is selected from the group consisting of mitomycin, bleomycin, doxorubicin and bendamustine. In some embodiments, the DNA-damaging agent induces expression of CD137 on an immune cell of the subject. In some embodiments, the DNA-damaging agent induces expression of CD137L on a cancer cell of the subject. In some embodiments, the DNA-damaging agent induces expression of CD137 on an immune cell of the subject and induces expression of CD137L on a cancer cell of the subject.

In some embodiments, there is provided a method of treating a cancer (e.g., B-cell lymphoma) in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4, and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7; and (b) an effective amount of a DNA-damaging agent (e.g., bendamustine) that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 8, and/or a VL comprises the amino acid sequence of SEQ ID NO: 9. In some embodiments, the DNA-damaging agent is a DNA chelator or an alkylating agent. In some embodiments, the DNA-damaging agent is selected from the group consisting of mitomycin, bleomycin, doxorubicin and bendamustine. In some embodiments, the DNA-damaging agent induces expression of CD137 on an immune cell of the subject. In some embodiments, the DNA-damaging agent induces expression of CD137L on a cancer cell of the subject. In some embodiments, the DNA-damaging agent induces expression of CD137 on an immune cell of the subject and induces expression of CD137L on a cancer cell of the subject.

In some embodiments, there is provided a method of treating a cancer (e.g., B-cell lymphoma) in a subject, comprising administering to the subject: (a) an effective amount of an anti-CD137 antibody that specifically binds to an extracellular domain of human CD137, wherein the anti-CD137 antibody comprises a VH and a VL, wherein the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14, and wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 17; and (b) an effective amount of a DNA-damaging agent (e.g., bendamustine) that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 18, and/or a VL comprises the amino acid sequence of SEQ ID NO: 19. In some embodiments, the DNA-damaging agent is a DNA chelator or an alkylating agent. In some embodiments, the DNA-damaging agent is selected from the group consisting of mitomycin, bleomycin, doxorubicin and bendamustine. In some embodiments, the DNA-damaging agent induces expression of CD137 on an immune cell of the subject. In some embodiments, the DNA-damaging agent induces expression of CD137L on a cancer cell of the subject. In some embodiments, the DNA-damaging agent induces expression of CD137 on an immune cell of the subject and induces expression of CD137L on a cancer cell of the subject.

In some embodiments, there is provided a method of treating a cancer (e.g., B-cell lymphoma) in a subject, comprising administering to the subject: (a) an effective amount of an anti-CD137 antibody that specifically binds to an extracellular domain of human CD137, wherein the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24, and wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27; and (b) an effective amount of a DNA-damaging agent (e.g., bendamustine) that induces expression of CD137 on an immune cell (e.g., CD8+ T cells, Treg cells, NK cells and/or NK-T cells) and/or induces expression of CD137L on a cancer cell of the subject. In some embodiments, the anti-CD137 antibody comprises a VH comprising the amino acid sequence of SEQ ID NO: 28, and/or a VL comprises the amino acid sequence of SEQ ID NO: 29. In some embodiments, the DNA-damaging agent is a DNA chelator or an alkylating agent. In some embodiments, the DNA-damaging agent is selected from the group consisting of mitomycin, bleomycin, doxorubicin and bendamustine. In some embodiments, the DNA-damaging agent induces expression of CD137 on an immune cell of the subject. In some embodiments, the DNA-damaging agent induces expression of CD137L on a cancer cell of the subject. In some embodiments, the DNA-damaging agent induces expression of CD137 on an immune cell of the subject and induces expression of CD137L on a cancer cell of the subject.

The anti-CD137 antibody and the CD137-inducing agent (e.g., cytokine, HDAC inhibitor, or DNA-damaging agent) may be administered in combination with one or more additional therapeutic agents or therapies. In some embodiment, the anti-CD137 antibody and the CD137-inducing agent (e.g., cytokine, HDAC inhibitor, or DNA-damaging agent) are administered in combination with one or more additional therapeutic agents for separate, sequential or simultaneous administration. The term “additional therapeutic agent” refers to any therapeutic agent other than an anti-CD137 antibody or a CD137-inducing agent (e.g., cytokine, HDAC inhibitor, or DNA-damaging agent) provided herein. Exemplary additional therapeutic agents or therapies include, for example, chemotherapeutic agents, immunotherapeutic agents, and hormone therapeutic agents. In some embodiments, the one or more additional therapeutic agents are selected from the group consisting of selected from the group consisting of viral gene therapy, immune checkpoint inhibitors, targeted therapies, radiation therapies, and chemotherapies.

In some embodiments, the anti-CD137 antibody and the CD137-inducing agent (e.g., cytokine, HDAC inhibitor, or DNA-damaging agent) are administered in combination with an anti-CD20 antibody. Exemplary anti-CD20 antibodies include, but are not limited to, rituximab, obinutuzumab, B-Lyl, 11B8, AT80, H147, 2C6, 2F2, 2H7 and GA101, biosimilars thereof, and derivatives thereof. In some embodiments, the anti-CD20 antibody is a type I anti-CD20 antibody. In some embodiments, the anti-CD20 antibody is a type II anti-CD20 antibody. In some embodiments, art recognized anti-CD20 antibodies can be used. For example, the anti-CD-20 antibodies disclosed in U.S. Pat. No. 7,879,984, WO2005/044859, WO2004/035607, WO2005/103081, WO2004/056312, WO2007/031875, and WO2015/095410 can be used in the methods disclosed herein. The teachings of each of the aforementioned publications are hereby incorporated by reference. In some embodiments, the antibodies that compete with any of these art-recognized antibodies for binding to CD-20 also can be used. In some embodiments, the anti-CD20 antibody is rituximab.

In some embodiments, there is provided a method of treating a cancer (e.g., B-cell lymphoma) in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1: (b) an effective amount of a DNA-damaging agent that that induces expression of CD137 on an immune cell (e.g., CD8+ T cells, Treg cells, NK cells and/or NK-T cells) and/or induces expression of CD137L on a cancer cell of the subject; and (c) an effective amount of an anti-CD20 antibody. In some embodiments, the anti-CD20 antibody is rituximab. In some embodiments, the DNA-damaging agent is a DNA chelator or an alkylating agent. In some embodiments, the DNA-damaging agent is selected from the group consisting of mitomycin, bleomycin, doxorubicin and bendamustine. In some embodiments, the DNA-damaging agent induces expression of CD137 on an immune cell of the subject. In some embodiments, the DNA-damaging agent induces expression of CD137L on a cancer cell of the subject. In some embodiments, the DNA-damaging agent induces expression of CD137 on an immune cell of the subject and induces expression of CD137L on a cancer cell of the subject.

In some embodiments, there is provided a method of treating a B-cell lymphoma in a subject, comprising administering to the subject: (a) an effective amount of any one of the anti-CD137 antibodies described herein; (b) an effective amount of bendamustine; and (c) an effective amount of an anti-CD20 antibody. In some embodiments, the anti-CD20 antibody is rituximab, a biosimilar thereof, or a derivative thereof. In some embodiments, the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4, and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7. In some embodiments, the anti-CD137 antibody comprises a VH comprising the amino acid sequence of SEQ ID NO: 8, and/or a VL comprises the amino acid sequence of SEQ ID NO: 9. In some embodiments, the anti-CD137 antibody comprises a heavy chain and a light chain, and wherein the heavy chain comprises the amino acid sequence of SEQ ID NO: 10, and/or the light chain comprises the amino acid sequence of SEQ ID NO: 11.

In some embodiments, the anti-CD137 antibody and the CD137-inducing agent (e.g., cytokine, HDAC inhibitor, or DNA-damaging agent) are administered in combination with an immune checkpoint inhibitor. Immune checkpoint inhibitors are compounds that inhibit the activity of control mechanisms of the immune system. Immune system checkpoints, or immune checkpoints, are inhibitory pathways in the immune system that generally act to maintain self-tolerance or modulate the duration and amplitude of physiological immune responses to minimize collateral tissue damage. Checkpoint inhibitors can inhibit an immune system checkpoint by stimulating the activity of a stimulatory checkpoint molecule, or inhibiting the activity of an inhibitory checkpoint molecule in the pathway. Immune system checkpoint molecules include, but are not limited to, cytotoxic T-lymphocyte antigen 4 (CTLA-4), programmed cell death 1 protein (PD-1), programmed cell death 1 ligand 1 (PD-L1), programmed cell death 1 ligand 2 (PD-L2), lymphocyte activation gene 3 (LAG3), B7-1, B7-H3, B7-H4, T cell membrane protein 3 (TIM3), B- and T-lymphocyte attenuator (BTLA), V-domain immunoglobulin (Ig)-containing suppressor of T-cell activation (VISTA), Killer-cell immunoglobulin-like receptor (KIR), and A2A adenosine receptor (A2aR). As such, checkpoint inhibitors include antagonists of CTLA-4, PD-1, PD-L1, PD-L2, LAG3, B7-1, B7-H3, B7-H4, BTLA, VISTA, KIR, A2aR, or TIM3. For example, antibodies that bind to CTLA-4, PD-1, PD-L1, PD-L2, LAG3, B7-1, B7-H3, B7-H4, BTLA, VISTA, KIR, A2aR, or TIM3 and antagonize their function are checkpoint inhibitors. Moreover, any molecule (e.g., peptide, nucleic acid, small molecule, etc.) that inhibits the inhibitory function of an immune system checkpoint is a checkpoint inhibitor.

In some embodiments, the immune checkpoint inhibitor is an antibody that specifically binds to an immune checkpoint molecule. In some embodiments, the immune checkpoint inhibitor is selected from the group consisting of an anti-PD-1 antibody, an anti-PD-L1 antibody, and an anti-CTLA-4 antibody.

In some embodiments, the immune checkpoint inhibitor is an anti-PD−1 antibody. Exemplary anti-PD-1 antibodies include, but are not limited to, 2E5 (Cstone Pharmaceuticals), tislelizumab (BGB-A317), BGB-108, STI-A1110, AM0001, BI 754091, sintilimab (1B1308), cetrelimab (JNJ-63723283), toripalimab (JS-001), camrelizumab (SHR-1210, INCSHR-1210, HR-301210), MEDI-0680 (AMP-S14), MGA-012 (INCMGA 0012), nivolumab (BMS-936558, MDX1106, ONO-4538), spartalizumab (PDROOI), pembrolizumab (MK-3475, SCH 900475), PF-06801591, cemiplimab (REGN-2810, REGEN2810), dostarlimab (TSR-042, ANB011), pidilizumab (CT-011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021. PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-01, BY-24.3, CB-201, IBI-319, ONCR-177, Max-1, CS-4100, JBI-426, CCC-0701, CCX-4503, biosimilars thereof, and derivatives thereof. In some embodiments, the antibodies that compete with any of these art-recognized antibodies for binding to PD-1 also can be used. In some embodiments, the immune checkpoint inhibitor is 2E5, 2E5 and related anti-PD-1 antibodies have been described, for example, in CN107840887A, which is incorporated herein by reference in its entirety. In some embodiments, the immune checkpoint inhibitor is toripalimab. Toripalimab and related anti-PD−1 antibodies have been described, for example, in U.S. Ser. No. 10/066,013B2, which is incorporated herein by reference in its entirety.

In some embodiments, the immune checkpoint inhibitor is an anti-PD-L1 antibody. Exemplary anti-PD-L1 antibodies include, but are not limited to, atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, FS-118, biosimilars thereof, and derivatives thereof. In some embodiments, the antibodies that compete with any of these art-recognized antibodies for binding to PD-L1 also can be used. In some embodiments, the immune checkpoint inhibitor is atezolizumab.

In some embodiments, the immune checkpoint inhibitor is an anti-CTLA-4 antibody. Exemplary anti-CTLA-4 antibodies include, but are not limited to, ipilimumab (IBI310, BMS-734016, MDX010, MDX-CTLA4, MEDI4736), tremelimumab (CP-675, CP-675,206), APL-509, AGEN1884, and CS1002, AGEN1181, Abatacept (Orencia, BMS-188667, RG2077), BCD-145, ONC-392, ADU-1604, REGN4659, ADG116, KN044, KN046, biosimilars thereof and derivatives thereof. In some embodiments, art recognized anti-CTLA-4 antibodies can be used. For example, the anti-CTLA-4 antibodies disclosed in: WO2019/149281, U.S. Pat. No. 8,119,129, WO 01/14424, WO 98/42752; WO 00/37504 (CP675,206, also known as tremelimumab; fonnerly ticilimumab), U.S. Pat. No. 6,207,156; WO2001014424, WO2000037504, and U.S. Pat. No. 8,017,114; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. In some embodiments, the antibodies that compete with any of these art-recognized antibodies for binding to CTLA-4 also can be used. In some embodiments, the anti-CTLA-4 antibody is ADG116. ADG116 (also known as TY21580) and related anti-CTLA-4 antibodies have been described, for example, in WO2019/149281, which is incorporated herein by reference in its entirety.

In some embodiments, there is provided a method of treating a cancer (e.g., melanoma) in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; (b) an effective amount of a cytokine that that induces expression of CD137 on an immune cell (e.g., CD8+ T cells, Treg cells, NK cells and/or NK-T cells) and/or induces expression of CD137L on a cancer cell of the subject; and (c) an effective amount of an anti-PD-1 antibody. In some embodiments, the anti-PD-1 antibody is 2E5. In some embodiments, the cytokine induces expression of CD137 on an immune cell of the subject. In some embodiments, the cytokine induces expression of CD137L on a cancer cell of the subject. In some embodiments, the cytokine induces expression of CD137 on an immune cell of the subject and induces expression of CD137L on a cancer cell of the subject. In some embodiments, the cytokine s selected from the group consisting of IL-2, IL-12, IL-10 and INFγ. In some embodiments, the cytokine is IL-2. In some embodiments, the IL-2 is administered at a dose of no more than about 2.8×10⁶ IU/m², e.g., about 7.2×10⁴ IU/kg or about 2.8×10⁶ IU/m².

In some embodiments, there is provided a method of treating a melanoma in a subject, comprising administering to the subject: (a) an effective amount of any one of the anti-CD137 antibodies described herein; (b) an effective amount of IL-2; and (c) an effective amount of an anti-PD-1 antibody. In some embodiments, the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4, and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7. In some embodiments, the anti-CD137 antibody comprises a VH comprising the amino acid sequence of SEQ ID NO: 8, and/or a VL comprises the amino acid sequence of SEQ ID NO: 9. In some embodiments, the anti-CD137 antibody comprises a heavy chain and a light chain, and wherein the heavy chain comprises the amino acid sequence of SEQ ID NO: 10, and/or the light chain comprises the amino acid sequence of SEQ ID NO: 11.

Cancer treatments can be evaluated by, e.g., tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

The anti-CD137 antibodies and the CD137-inducing agents (e.g., cytokine, HDAC inhibitor, or DNA-damaging agent) provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The antibodies and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art. In some embodiments, the anti-CD137 antibody is administered intravenously. In some embodiments, the CD137-inducing agent (e.g., cytokine, HDAC inhibitor, or DNA-damaging agent) is administered intravenously.

In some embodiments, the anti-CD137 antibody is administered at a flat dose. In some embodiments, the anti-CD137 antibody is administered at a dose of no more than any one of 500 mg, 475 mg, 450 mg, 425 mg, 400 mg, 390 mg, 380 mg, 370 mg, 360 mg, 350 mg, 340 mg, 330 mg, 320 mg, 310 mg, 300 mg, 275 mg, 250 mg, 225 mg, 200 mg, 175 mg, 150 mg, or 125 mg. In some embodiments, the dose of the anti-CD137 antibody is within any one of the following ranges, wherein the ranges have an upper limit of any one of: 500 mg, 475 mg, 450 mg, 425 mg, 400 mg, 390 mg, 380 mg, 370 mg, 360 mg, 350 mg, 340 mg, 330 mg, 320 mg, 310 mg, 300 mg, 275 mg, 250 mg, 225 mg, 200 mg, 175 mg, or 150 mg, and an independently selected lower limit of any one of 475 mg, 450 mg, 425 mg, 400 mg, 390 mg, 380 mg, 370 mg, 360 mg, 350 mg, 340 mg. 330 mg, 320 mg, 310 mg, 300 mg, 275 mg, 250 mg, 225 mg, 200 mg, 175 mg, 150 mg, or 125 mg, and wherein the lower limit is less than the upper limit. In some embodiments, the anti-CD137 antibody is administered at a dose of any one of about 150 mg to about 200 mg, about 150 mg to about 300 mg, about 150 mg to about 400 mg, about 150 mg to about 500 mg, about 125 mg to about 200 mg, about 200 mg to about 300 mg, about 300 mg to about 400 mg, about 400 mg to about 500 mg, about 125 mg to about 300 mg, about 300 mg to about 500 mg, about 200 mg to about 400 mg, about 125 mg to about 250 mg, about 250 mg to about 500 mg, about 250 mg to about 400 mg, about 125 mg to about 400 mg, about 200 mg to about 500 mg, or about 125 mg to about 500 mg. The doses described herein may refer to a suitable dose for a human, or an equivalent dose for the specific species of the subject. In some embodiments, the anti-CD137 antibody is administered at a dose equivalent to no more than 500 mg (such as no more than 400 mg/kg) for a human subject. In some embodiments, the anti-CD137 antibody is administered at a dose of about 125 mg to about 500 mg, such as about any one of 125, 150, 200, 250, 300, 350, 400, 450 or 500 mg.

In some embodiments, the anti-CD137 antibody is administered at a dose of no more than any one of 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.8 mg/kg, 0.6 mg/kg, 0.5 mg/kg, 0.4 mg/kg, 0.3 mg/kg, 0.2 mg/kg, 0.1 mg/kg, 0.08 mg/kg, 0.05 mg/kg. 0.04 mg/kg, or 0.03 mg/kg. In some embodiments, the dose of the anti-CD137 antibody is within any one of the following ranges, wherein the ranges have an upper limit of any one of: 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.8 mg/kg, 0.6 mg/kg, 0.5 mg/kg, 0.4 mg/kg, 0.3 mg/kg, 0.2 mg/kg, 0.1 mg/kg, 0.08 mg/kg, 0.05 mg/kg, or 0.04 mg/kg, and an independently selected lower limit of any one of 9 mg/kg, 8 mg/kg. 7 mg/kg, 6 mg/kg, 5 mg/kg, 4 mg/kg, 3 mg/kg, 2 mg/kg, 1 mg/kg, 0.8 mg/kg, 0.6 mg/kg, 0.5 mg/kg, 0.4 mg/kg, 0.3 mg/kg, 0.2 mg/kg, 0.1 mg/kg, 0.08 mg/kg, 0.05 mg/kg, 0.04 mg/kg, or 0.03 mg/kg, and wherein the lower limit is less than the upper limit. In some embodiments, the anti-CD137 antibody is administered at a dose of any one of about 0.03 mg/kg to about 10 mg/kg, about 0.1 mg/kg to about 10 mg/kg, about 0.3 mg/kg to about 10 mg/kg, about 1 mg/kg to about 10 mg/kg, about 3 mg/kg to about 10 mg/kg, about 5 mg/kg to about 10 mg/kg, about 0.03 mg/kg to about 0.1 mg/kg, about 0.1 mg/kg to about 0.3 mg/kg, about 0.3 mg/kg to about 1 mg/kg, about 1 mg/kg to about 3 mg/kg, about 3 mg/kg to about 5 mg/kg, about 0.1 mg/kg to about 3 mg/kg, or about 1 mg/kg to about 5 mg/kg. The doses described herein may refer to a suitable dose for a human, or an equivalent dose for the specific species of the subject. In some embodiments, the anti-CD137 antibody is administered at a dose equivalent to about 0.1 mg/kg to about 10 mg/kg (such as about 3 mg/kg to about 8 mg/kg, or about 5 mg/kg to about 10 mg/kg) for a human subject. In some embodiments, the anti-CD137 antibody is administered at a dose equivalent to no more than 10 mg/kg (such as no more than 8 mg/kg, or no more than 5 mg/kg) for a human subject. In some embodiments, the anti-CD137 antibody is administered at a dose of about 0.03 mg/kg to about 10 mg/kg, such as about any one of 0.03, 0.1, 0.3, 1, 3, 5, 8 or 10 mg/kg.

The effective amount of the anti-CD137 antibody may be administered in a single dose or in multiple doses. For methods that comprises administration of the anti-CD137 antibody in multiple doses, exemplary dosing frequencies include, but are not limited to, weekly, weekly without break, weekly for two out of three weeks, weekly for three out of four weeks, once every three weeks, once every two weeks, monthly, every six months, yearly, etc. In some embodiments, the anti-CD137 antibody is administered about weekly, once every 2 weeks, or once every 3 weeks. In some embodiments, the intervals between each administration are less than about any of 3 years, 2 years, 12 months, 11 months, 10 months, 9 months, 8 months, 7 months, 6 months, 5 months, 4 months, 3 months, 2 months, 1 month, 4 weeks, 3 weeks, 2 weeks, or 1 week. In some embodiments, the intervals between each administration are more than about any of 1 week, 2 weeks, 3 weeks, 4 weeks, 1 month, 2 months, 3 months, 4 months, 5 months, 6 months, 7 months, 8 months, 9 months, 10 months, 11 months, 12 months, 2 years, or 3 years. In some embodiments, there is no break in the dosing schedule.

In some embodiments, the anti-CD137 antibody is administered at a low frequency, for example, any one of no more frequent than once per week, once every other week, once per three weeks, once per month, once per 2 months, once per 3 months, once per 4 months, once per 5 months, once per 6 months, once per 7 months, once per 8 months, once per 9 months, once per 10 months, once per 11 months, once per year, or less. In some embodiments, the anti-CD137 antibody is administered in a single dose. In some embodiments, the anti-CD137 antibody is administered about once every three weeks.

In some embodiments, the anti-CD137 antibody is administered at a dose of no more than 500 mg, such as no more than any one of 400 mg. 350 mg, 300 mg, 250 mg, 200 mg, 150 mg or 125 mg once every three weeks. In some embodiments, the anti-CD137 antibody is administered at a dose of about 125 mg to about 500 mg, such as about any one of 125 mg, 200 mg, 250 mg, 300 mg, 350 mg, or 400 mg, once every three weeks.

In some embodiments, the anti-CD137 antibody is administered at a dose of no more than 10 mg/kg, such as no more than any one of 8 mg/kg, 5 mg/kg, 3 mg/kg, 2 mg/kg, or 1 mg/kg once every three weeks. In some embodiments, the anti-CD137 antibody is administered at a dose of about 0.03 mg/kg to about 10 mg/kg, such as about any one of 0.03, 0.1, 0.3, 1, 3, 5, 8, or 10 mg/kg, once every three weeks.

Suitable dosages for the CD137-inducing agent (e.g., cytokine, HDAC inhibitor, or DNA-damaging agent) depend on factors such as the nature of the CD137-inducing agent, type of the cancer being treated, and the routes of administration. Exemplary doses of the CD137-inducing agent (e.g., cytokine, HDAC inhibitor, or DNA-damaging agent) include, but are not limited to, about any one of 1 mg/m², 5 mg/m², 10 mg/m², 20 mg/m², 50 mg/m², 100 mg/m², 200 mg/m², 300 mg/m², 400 mg/m², 500 mg/m², 750 mg/m², 1000 mg/m², or more. In some embodiments, the dose of the CD137-inducing agent (e.g., cytokine, HDAC inhibitor, or DNA-damaging agent) is included in any one of the following ranges: about 1 to about 5 mg/m², about 5 to about 10 mg/m², about 10 to about 20 mg/m², about 20 to about 50 mg/m², about 50 to about 100 mg/m², about 100 mg/m² to about 200 mg/m², about 200 to about 300 mg/m², about 300 to about 400 mg/m², about 400 to about 500 mg/m², about 500 to about 750 mg/m², or about 750 to about 1000 mg/m². In some embodiments, the dose of the CD137-inducing agent (e.g., cytokine, HDAC inhibitor, or DNA-damaging agent) is about any one of 1 μg/kg, 2 μg/kg, 5 μg/kg, 10 μg/kg, 20 μg/kg, 50 μg/kg, 0.1 mg/kg, 0.2 mg/kg, 0.3 mg/kg, 0.4 mg/kg, 0.5 mg/kg, 1 mg/kg, 2 mg/kg, 5 mg/kg, 10 mg/kg, 20 mg/kg, 50 mg/kg, 100 mg/kg, or more. In some embodiments, the dose of the CD137-inducing agent (e.g., cytokine, HDAC inhibitor, or DNA-damaging agent) is any one of about 1 μg/kg to about 5 μg/kg, about 5 μg/kg to about 10 μg/kg, about 10 μg/kg to about 50 μg/kg, about 50 μg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 1 mg/kg, about 1 mg/kg to about 5 mg/kg, about 5 mg/kg to about 10 mg/kg, about 10 mg/kg to about 20 mg/kg, about 20 mg/kg to about 50 mg/kg, about 50 mg/kg to about 100 mg/kg, or about 1 mg/kg to about 100 mg/kg. In some embodiments, the dose of the CD137-inducing agent (e.g., cytokine, HDAC inhibitor, or DNA-damaging agent) is about any one of 1 μg, 10 μg, 50 μg, 100 μg, 500 μg, 1 mg, 10 mg, 50 mg, 100 mg, 500 mg or 1000 mg. In some embodiments, the dose of the CD137-inducing agent (e.g., cytokine, HDAC inhibitor, or DNA-damaging agent) is any one of about 1 μg to about 10 μg, about 10 μg to about 50 10 μg, about 50 μg to about 100 μg, about 100 μg to about 500 μg, about 500 μg to about 1 mg, about 1 mg to about 5 mg, about 5 mg to about 10 mg, about 10 mg to about 25 mg, about 25 mg to about 50 mg, about 50 mg to about 100 mg, about 100 mg to about 500 mg, about 500 mg to about 1000 mg, about 1 μg to about 1 mg, about 1 mg to about 1000 mg, or about 1 μg to about 1000 mg.

In some embodiments, the CD137-inducing agent (e.g., cytokine, HDAC inhibitor, or DNA-damaging agent) is administered daily. In some embodiments, the CD137-inducing agent (e.g., cytokine, HDAC inhibitor, or DNA-damaging agent) is administered is administered at least about any one of 1×, 2×, 3×, 4×, 5×, 6×, or 7× (i.e., daily) a week. In some embodiments, the CD137-inducing agent (e.g., cytokine, HDAC inhibitor, or DNA-damaging agent) is administered weekly. In some embodiments, the CD137-inducing agent (e.g., cytokine, HDAC inhibitor, or DNA-damaging agent) is administered weekly without break; weekly, two out of three weeks; weekly three out of four weeks; once every two weeks; once every 3 weeks; once every 4 weeks; once every 6 weeks; once every 8 weeks, monthly, or every two to 12 months. In some embodiments, the intervals between each administration are less than about any one of 6 months, 3 months, 1 month, 20 days, 15, days, 12 days, 10 days, 9 days, 8 days, 7 days, 6 days, 5 days, 4 days, 3 days, 2 days, or 1 day. In some embodiments, the intervals between each administration are more than about any one of 1 month, 2 months, 3 months, 4 months, 5 months, 6 months, 8 months, or 12 months. In some embodiments, there is no break in the dosing schedule. In some embodiments, the interval between each administration is no more than about a week. In some embodiments, the CD137-inducing agent (e.g., cytokine, HDAC inhibitor, or DNA-damaging agent) is administered with the same dosing schedule as the anti-CD137 antibody. In some embodiments, the CD137-inducing agent (e.g., cytokine, HDAC inhibitor, or DNA-damaging agent) is administered with a different dosing schedule as the anti-CD137 antibody.

In some embodiments, the IL-2 is administered at a continuous low dose. In some embodiments, the IL-2 is administered at least daily. In some embodiments, the IL-2 is administered twice daily. In some embodiments, the IL-2 is administered three times per day, i.e., every 8 hours. In some embodiments, the IL-2 is administered at least daily for at least 7, 8, 9, 10, 11, 12, 13, 14, 15, 16, 17, 18, 19, 20, 21, 22, 23, 24, 25, 26, 27, 28 days or more. In some embodiments, the IL-2 is administered twice daily for about 14 days to about 28 days. In some embodiments, the IL-2 is administered at a dose of no more than about any one of 2.8×10⁶, 2.5×10⁶, 2×10⁶, 1.5×10⁶, 1×10⁶, 9×10⁵, 8×10⁵, 7×10⁵, 6×10⁵, 5×10⁵, 4×10⁵, 3-10⁵, 2×10⁵, 1.4×10⁵ International Units (IU)/m². In some embodiments, the IL-2 is administered at a dose of about any one of 1.4×10⁵ IU/m² to 5-10 IU/m², 5×10 IU/m² to 1×10⁶ IU/m², 1×10⁶ IU/m² to 1.5×10⁶ IU/m², 1×10⁶ IU/m² to 2×10⁶ IU/m², 1×10⁶ IU/m² to 2.8×10⁶ IU/m², 1.4×10⁶ IU/m² to 2.8×10⁶ IU/m², 7×10⁵ IU/m² to 2.8×10⁶ IU/m², or 1.4×10⁵ IU/m² to 2.8×10⁶ IU/m². In some embodiments, the IL-2 is administered at a dose of no more than about any one of 8×10⁴, 7.2×10⁴, 6×10⁴, 5×10⁴, 4×10⁴, 3×10⁴, 2×10⁴, 1×10⁴, 9×10³, 8×10³, 7×10³, 6×10³, or 5×10³ IU/kg. In some embodiments, the IL-2 is administered at a dose of about any one of 5×10³ IU/kg to 1×10⁴ IU/kg, 1×10⁴ IU/kg to 4×10⁴ IU/kg, 4×10⁴ IU/kg to 8×10⁴ IU/kg, 5×10³ IU/kg to 8×10⁴ IU/kg, 5×10³ IU/kg to 5×10⁴ IU/kg, 5×10³ IU/kg to 7.2×10⁴ IU/kg, 1×10⁴ IU/kg to 7.2×10⁴ IU/kg, or 7.2×10³ IU/kg to 7.2×10⁴ IU/kg. In some embodiments, the IL-2 is administered twice or three times daily at a dose of about 7.2×10⁴ ILU/kg. In some embodiments, the IL-2 is administered twice or three times daily at a dose of about 2.8×10⁶ IU/m². The doses described herein may refer to a suitable dose for a mouse, or an equivalent dose for the specific species of the subject (e.g., human).

In some embodiments, the IL-2 is administered at a low frequency. In some embodiments, the IL-2 is administered at a frequency of no more than about once every 2, 3, 4, 5, 6, 7 days or more. In some embodiments, the IL-2 is administered at a frequency of no more than once every three days. In some embodiments, the IL-2 is administered for no more than about any one of 14, 13, 12, 11, 10, 9, 8, 7, 6, 5, 4, or 3 doses. In some embodiments, the IL-2 is administered at a dose of at least about any one of 2.8×10⁶, 3×10⁶, 4×10⁶, 5×10⁶, 6×10⁶, 7×10⁶, 8×10⁶, 9×10⁶, 1×10⁷, 1.2×10⁷, or 1.4×10⁷ IU×m². In some embodiments, the IL-2 is administered at a dose of no more than about any one of 1.4×10⁷, 1.2×10⁷, 1×10⁷, 9×10⁶, 8×10⁶, 7×10⁶, 6×10⁶, 5×10⁶, 4×10⁶, 3×10⁶, or 2.8×10⁶ IU/m². In some embodiments, the IL-2 is administered at a dose of about any one of 2.8×10⁶ IU/m² to 5×10⁶ IU/m², 5×10⁶ IU/m² to 1×10⁷ IU/m², 2.8×10⁶ IU/m² to 7×10⁶ IU/m², 7×10⁶ IU/m² to 1.4×10⁷ IU/m², 7×10⁷ IU/m² to 1.4×10⁷ IU/m², 2.8×10⁶ IU/m² to 1×10⁷ IU/m², or 2.8×10⁶ IU/m² to 1.4×10⁷ IU/m². In some embodiments, the IL-2 is administered at a dose of at least about any one of 7.2×10⁴, 8×10⁴, 9×10⁴, 1×10⁵, 2×10⁵, 3×10⁵, 4×10⁵, 5×10⁵, 6×10⁵, or 7.2×10⁵ IU/kg. In some embodiments, the IL-2 is administered at a dose of no more than about any one of 7.2×10⁵, 6×10⁵, 5×10⁵, 4×10⁵, 3×10⁵, 2×10⁵, 1×10⁵, 9×10⁴, 8×10⁴, or 7.2×10⁴ IU/kg. In some embodiments, the IL-2 is administered at a dose of about any one of 7.2×10⁴ IU/kg to 1×10⁵ IU/kg, 1×10⁵ IU/kg to 3×10⁵ IU/kg, 3×10⁵ IU/kg to 7.2×10⁵ IU/kg, 7.2×10⁴ IU/kg to 2×10⁵ IU/kg, 1×10⁵ IU/kg to 7.2×10⁵ IU/kg, or 7.2×10⁴ IU/kg to 7.2×10⁵ IU/kg. In some embodiments, the IL-2 is administered once every three days at a dose of about 7.2×10⁵ IU/kg. In some embodiments, the IL-2 is administered once every three days at a dose of about 1.4×10⁷ IU/m². The doses described herein may refer to a suitable dose for a mouse, or an equivalent dose for the specific species of the subject (e.g., human).

In some embodiment, the DNA-damaging agent (e.g., bendamustine) is administered at a dose of about 12.5 mg/kg. In some embodiments, the DNA-damaging agent (e.g., bendamustine) is administered once daily. In some embodiments, the DNA-damaging agent is administered for at least about any one of 3, 4, 5, 6, 7, or more doses. The doses described herein may refer to a suitable dose for a mouse, or an equivalent dose for the specific species of the subject (e.g., human).

In some embodiments, the anti-CD137 antibody and the CD137-inducing agent (e.g., cytokine, HDAC inhibitor, or DNA-damaging agent) are administered sequentially, i.e., the anti-CD137 antibody is administered before or after the administration of the CD137-inducing agent (e.g., cytokine, HDAC inhibitor, or DNA-damaging agent). In some embodiments, the CD137-inducing agent (e.g., cytokine, HDAC inhibitor, or DNA-damaging agent) is administered prior to the administration of the anti-CD137 antibody. In some embodiments, the CD137-inducing agent (e.g., cytokine, HDAC inhibitor, or DNA-damaging agent) is administered no more than about any of 15 minutes. 30 minutes, 1 hour, 2 hours, 3 hours, 4 hours, 5 hours, 6 hours, 12 hours, or 24 hours prior to the administration of the anti-CD137 antibody. In some embodiments, the CD137-inducing agent (e.g., cytokine, HDAC inhibitor, or DNA-damaging agent) is administered about days or weeks (such as about any of 1 day, 2 days, 3 days, 4 days, 5 days, 6 days, 1 week, 2 weeks, 3 weeks, 4 weeks, or more) prior to the administration of the anti-CD137 antibody. In some embodiments, the CD137-inducing agent (e.g., cytokine, HDAC inhibitor, or DNA-damaging agent) is administered after the administration of the anti-CD137 antibody. In some embodiments, the CD137-inducing agent (e.g., cytokine, HDAC inhibitor, or DNA-damaging agent) is administered no more than about any of 15 minutes, 30 minutes, 1 hour, 2 hours, 3 hours, 4 hours, 5 hours, 6 hours, 12 hours, or 24 hours after the administration of the anti-CD137 antibody. In some embodiments, the CD137-inducing agent (e.g., cytokine, HDAC inhibitor, or DNA-damaging agent) is administered about days or weeks (such as about any of 1 day, 2 days, 3 days, 4 days, 5 days, 6 days. 1 week, 2 weeks, 3 weeks. 4 weeks, or more) after the administration of the anti-CD137 antibody. In some embodiments, the anti-CD137 antibody and the CD137-inducing agent (e.g., cytokine, HDAC inhibitor, or DNA-damaging agent) are administered with one immediately after another (e.g., within 5 minutes or less between the two administrations). For example, in some embodiments, the CD137-inducing agent (e.g., cytokine, HDAC inhibitor, or DNA-damaging agent) is administered immediately before the administration of the anti-CD137 antibody. In some embodiments, the CD137-inducing agent (e.g., cytokine, HDAC inhibitor, or DNA-damaging agent) is administered immediately after the administration of the anti-CD137 antibody.

In some embodiments, the anti-CD137 antibody and the CD137-inducing agent (e.g., cytokine, HDAC inhibitor, or DNA-damaging agent) are administered simultaneously. In some embodiments, the anti-CD137 antibody and the CD137-inducing agent (e.g., cytokine, HDAC inhibitor, or DNA-damaging agent) are administered simultaneously via separate compositions. In some embodiments, the anti-CD137 antibody and the CD137-inducing agent (e.g., cytokine, HDAC inhibitor, or DNA-damaging agent) are administered as a single composition. In some embodiments, the anti-CD137 antibody and the CD137-inducing agent (e.g., cytokine, HDAC inhibitor, or DNA-damaging agent) are mixed prior to (such as immediately prior to, e.g., within less than about 10, 5, or 1 minutes before) the administration of the composition. In some embodiments, the composition comprising the anti-CD137 antibody and the CD137-inducing agent (e.g., cytokine, HDAC inhibitor, or DNA-damaging agent) is pre-made and stored for at least about 1 hours, 2 hours, 3 hours, 4 hours, 5 hours, 6 hours. 12 hours, 24 hours, 2 days, 3 days, 4 days, 5 days. 6 days, 7 days, 2 weeks, 3 weeks, or more prior to the administration.

In some embodiments, the anti-CD137 antibody and the CD137-inducing agent are administered for 2 or more cycles, such as about any one of 2, 3, 4, 5, 6, 7, 8, 9, 10, 11, 12, or more cycles. The administration of the anti-CD137 antibody and the CD137-inducing agent can be extended over an extended period of time, such as from about a week to about a month, from about a month to about a year, from about a year to about several years. In some embodiments, the anti-CD137 antibody and the CD137-inducing agent are administered over a period of at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more.

The methods described herein are useful for treating a variety of cancers. In some embodiments, the cancer is a solid cancer. In some embodiments, the cancer is a liquid cancer. A variety of cancers where CD137 is implicated, whether malignant or benign and whether primary or secondary, may be treated or prevented with a method provided by the disclosure. The methods are applicable to liquid or solid cancers of all stages, including stages, I, II, III, and IV, according to the American Joint Committee on Cancer (AJCC) staging groups. In some embodiments, the cancer is an/a: early stage cancer, non-metastatic cancer, primary cancer, advanced cancer, locally advanced cancer, metastatic cancer, cancer in remission, cancer in an adjuvant setting, or cancer in a neoadjuvant setting. In some embodiments, the cancer is localized resectable, localized unresectable, or unresectable. In some embodiments, the cancer has been refractory to prior therapy.

In some embodiments, the cancer is a liquid cancer. In some embodiments, the cancer is non-Hodgkin's lymphoma (NHL). In some embodiments, the NHL arises from B-lymphocytes. In some embodiments, the cancer is a B cell lymphoma. In some embodiments, the cancer is a diffuse large B-cell lymphoma (DLBCL).

In some embodiments, the cancer is T cell lymphoma (TCL). In some embodiments, the cancer is T-lymphoblastic lymphoma or leukemia. In some embodiments, the cancer is peripheral T-cell lymphoma. In some embodiments, the cancer is angioimmunoblastic T-cell lymphoma (AITL). In some embodiments, the cancer is extranodal natural killer/T-cell lymphoma, e.g., nasal type. In some embodiments, the cancer is enteropathy-associated intestinal T-cell lymphoma (EATL). In some embodiments, the cancer is lymph node/tonsil type of TCL. In some embodiments, the cancer is anaplastic large cell lymphoma (ALCL). In some embodiments, the cancer is peripheral T-cell lymphoma (PTCL).

In some embodiments, the cancer is multiple myeloma.

In some embodiments, the cancer is a solid cancer. In some embodiments, the cancer is selected from the group consisting of breast cancer, ovarian cancer, colorectal cancer, gastric cancer, melanoma, liver cancer, lung cancer, thyroid cancer, kidney cancer, brain cancer, cervical cancer, bladder cancer, and esophageal cancer. In some embodiments, the cancer is lung cancer, e.g., non-small cell lung cancer or NSCLC. In some embodiments, the cancer is melanoma.

The methods described herein are useful for various aspects of cancer treatment. In some embodiments, there is provided a method of inhibiting cell proliferation (such as tumor growth) in an individual, comprising administering to the individual an effective amount of any one of the anti-CD137 antibodies described herein and an effective amount of any one of the CD137-inducing agents described herein. In some embodiments, at least about 10% (including for example at least about any of 20%, 30%, 40%, 60%, 70%, 80%, 90%, 95% or more) cell proliferation is inhibited.

In some embodiments, there is provided a method of inhibiting tumor metastasis in an individual, comprising administering to the individual an effective amount of any one of the anti-CD137 antibodies described herein and an effective amount of any one of the CD137-inducing agents described herein. In some embodiments, at least about 10% (including for example at least about any of 20%, 30%, 40%, 60%, 70%, 80%, 90%, 95% or more) metastasis is inhibited.

In some embodiments, there is provided a method of reducing (such as eradicating) pre-existing tumor metastasis (such as metastasis to the lymph node) in an individual, comprising administering to the individual an effective amount of any one of the anti-CD137 antibodies described herein and an effective amount of any one of the CD137-inducing agents described herein. In some embodiments, at least about 10% (including for example at least about any of 20%, 30%, 40%, 60%, 70%, 80%, 90%, 95% or more) metastasis is reduced.

In some embodiments, there is provided a method of reducing incidence or burden of preexisting tumor metastasis (such as metastasis to the lymph node) in an individual, comprising administering to the individual an effective amount of any one of the anti-CD137 antibodies described herein and an effective amount of any one of the CD137-inducing agents described herein.

In some embodiments, there is provided a method of reducing tumor size in an individual, comprising administering to the individual an effective amount of any one of the anti-CD137 antibodies described herein and an effective amount of any one of the CD137-inducing agents described herein. In some embodiments, the method reduces tumor size by at least about 10% (including for example at least about any of 20%, 30%, 40%, 60%, 70%, 80%, 90%, 95% or more).

In some embodiments, there is provided a method of prolonging time to disease progression of cancer in an individual, comprising administering to the individual an effective amount of any one of the anti-CD137 antibodies described herein and an effective amount of any one of the CD137-inducing agents described herein. In some embodiments, the method prolongs the time to disease progression by at least any of 1, 2, 3, 4, 5, 6, 7, 8, 9, 10, 11, 12, 16, 20, 24, 28, 32, 36, or more weeks.

In some embodiments, there is provided a method of prolonging survival (e.g., overall survival or progression-free survival) of an individual having cancer, comprising administering to the individual an effective amount of any one of the anti-CD137 antibodies described herein and an effective amount of any one of the CD137-inducing agents described herein. In some embodiments, the method prolongs the survival of the individual by at least any of 1, 2, 3, 4, 5, 6, 7, 8, 9, 10, 11, 12, 18, or 24 months.

In some embodiments, there is provided a method of alleviating one or more symptoms in an individual having cancer, comprising administering to the individual an effective amount of any one of the anti-CD137 antibodies described herein and an effective amount of any one of the CD137-inducing agents described herein.

In some embodiments, there is provided a method of improving the quality of life in an individual having cancer, comprising administering to the individual an effective amount of any one of the anti-CD137 antibodies described herein and an effective amount of any one of the CD137-inducing agents described herein.

Also provided are compositions of any one of the anti-CD137 antibodies described herein and any one of the CD137-inducing agents described herein for use in the methods described in this section, and use of any one of the anti-CD137 antibodies described herein and any one of the CD137-inducing agents described herein in the manufacture of a medicament for treating cancer.

CD137-inducing Agents

The methods described herein comprise administration of an agent (also referred herein as “CD137-inducing agent”) that induces expression of CD137 on an immune cell and/or induces expression of CD137L on a cancer cell of the subject.

In some embodiments, the CD137-inducing agent induces expression of CD137 on immune cells. Exemplary immune cells include, but are not limited to, peripheral blood mononuclear cells (PBMCs), CD8+ T cells, regulatory T (Treg) cells, natural killer (NK) cells, and NK-T cells. In some embodiments, the CD137-inducing agent induces expression of CD137 by at least about any one of 5, 10, 20, 50, 100, 200, 500, 1000, or more folds. In some embodiments, the CD137-inducing agent increases the percentage of CD137+ immune cells (e.g., CD8+ T cells, Treg cells, NK cells, and/or NK-T cells) in a sample (e.g., blood sample or tumor biopsy) of the subject by at least about any one of 5, 10, 20, 50, 100, 200, 500, 1000, or more folds. In some embodiments, the percentage of CD137-expressing immune cells (e.g., CD8+ T cells, Treg cells, NK cells, and/or NK-T cells) in the subject after treatment with the CD137-inducing agent is at least about any one of 10%, 15%, 20%, 25%, 30%, 35%, 40%, or more. Expression of CD137 can be assessed at RNA or protein level using known methods in the art, including, for example, quantitative polymerase chain reaction (qPCR), RNA sequencing, Western blotting, and immunohistochemical staining.

In some embodiments, the CD137-inducing agent induces expression of CD137L on cancer cells. In some embodiments, the CD137-inducing agent induces expression of CD137L by at least about any one of 5, 10, 20, 50, 100, 200, 500, 1000, or more folds. In some embodiments, the CD137-inducing agent increases the percentage of CD137L+ cancer cells in a sample (e.g., tumor biopsy) of the subject by at least about any one of 5, 10, 20, 50, 100, 200, 500, 1000, or more folds. In some embodiments, the percentage of CD137L-expressing cancer cells in the subject after treatment with the CD137-inducing agent is at least about any one of 10%, 15%, 20%, 25%, 30%, 35%, 40%, or more. Expression of CD137L can be assessed at RNA or protein level using known methods in the art, including, for example, quantitative polymerase chain reaction (qPCR), RNA sequencing. Western blotting, and immunohistochemical staining.

In some embodiments, the CD137-inducing agent is a cytokine. Exemplary cytokines include, but are not limited to IL-2, IL-12, IL-10 and INFγ. In some embodiments, the cytokine is a wild-type cytokine, a native cytokine, a recombinant cytokine, a chemically modified cytokine (e.g., a PEGylated cytokine, a deglycosylated cytokine, etc.), or a cytokine analog. A “cytokine analog” refers to an engineered polypeptide having insertion(s), deletion(s), and/or substitution(s) of one or more amino acid residues while retaining substantially the same (e.g., at least about any one of 60%, 70%, 80%, 90%, 95%, or more) activity (e.g., receptor binding) as a wild-type cytokine. Typically, a cytokine analog has enhanced pharmacokinetic properties, such as stability and serum half-life, compared to a wild-type cytokine. In some embodiments, a cytokine analog has an amino acid sequence having at least about any one of 80%, 85%, 90%, 91%, 92%, 93%, 94%, 95%, 96%, 97%, 98%, 99%, or more identity to the amino acid sequence of a wildtype cytokine. In some embodiments, a cytokine analog about any one of 10, 9, 8, 7, 6, 5, 4, 3, 2, or 1 mutations (e.g., substitution, insertion and/or deletion) to the amino acid sequence of a wildtype cytokine.

In some embodiments, the CD137-inducing agent is IL-2. In some embodiments, the CD137-inducing agent is an agonist of IL-2 receptor. In some embodiments, the CD137-inducing agent is an IL-2Rβ-selective agonist. In some embodiments, the CD137-inducing agent is a long-acting IL-2 analog. In some embodiments, the CD137-inducing agent is a conjugate of IL-2 to a water-soluble polymer, such as PEG. Non-limiting examples of long acting, IL-2Rβ-selective agonists are described in WO 2012/065086, which is incorporated herein by reference in its entirety.

IL-2 or interleukin-2 is a cytokine that regulates the activities of lymphocytes. Native IL-2 is a protein of about 15.5-16 kDa, which functions by binding to IL-2 receptors. The IL-2 receptor is a complex having three chains: IL-2α (CD25), IL-2β (CD122) and IL-2γ (CD132), with each of IL-2Rα and IL-2Rβ having binding affinity for IL-2 while IL-2Rγ alone has no appreciable affinity. See, Theze et al. (1994) Immunol. Today 17(10):481-486. The IL-2 receptor (IL-2R) α subunit binds IL-2 with low affinity (Kd˜10⁻⁸ M). Interaction of IL-2 and CD25 alone does not lead to signal transduction but has the ability (when bound to the β and γ subunit) to increase the IL-2R affinity. Heterodimerization of the β and γ subunits of IL-2R is essential for signaling in T cells. IL-2 can signal either via intermediate-affinity dimeric CD122/CD132 IL-2R (Kd˜10⁻⁹ M) or via high-affinity trimeric CD25/CD122/CD132 IL-2R (Kd˜10⁻¹¹ M). Dimeric IL-2R is expressed by memory CD8+ T cells and NK cells, whereas regulatory T cells and activated T cells express high levels of trimeric IL-2R.

A high-dose IL-2 therapy, PROLEUKIN®, has been approved by the U.S. Food and Drug Administration (FDA) for treatment of patients with metastatic melanoma and renal cell carcinoma (RCC), with beneficial results in a subset of patients. However, vascular leak syndrome, hypotension, and liver toxicities associated with high-dose IL-2 have limited its use in cancer immunotherapy. In addition, high-dose IL-2 can expand potently suppressive CD4+CD25+ Foxp3+ Tregs in cancer patients, possibly limiting its efficacy as a monotherapy for cancer therapy.

In some embodiments, the IL-2 is a human IL-2. Human IL-2 (UniProt ID: P60568) is a glycosylated protein having 153 amino acids, including a signal peptide at amino acid residues 1-20. In some embodiments, the IL-2 is a wildtype human IL-2 comprising amino acid residues 21-153 of the amino acid sequence of SEQ ID NO: 43.

In some embodiments, the IL-2 is a functional variant of human IL-2.

human IL-2 amino acid sequence
SEQ ID NO: 43
MYRMQLLSCIALSLALVTNSAPTSSSTKKTQLQLE
HLLLDLQMILNGINNYKNPKLTRMLTFKFYMPKKA
TELKHLQCLEEELKPLEEVLNLAQSKNFHLRPRDL
ISNINVIVLELKGSETTFMCEYADETATIVEFLNR
WITFCQSIISTLT

In some embodiments, the IL-2 is an analog of human IL-2. In some embodiments, the IL-2 comprising the amino acid sequence of SEQ ID NO: 44. In some embodiments, the IL-2 is aldesleukin. Aldesleukin (e.g., PROLEUKIN®) is a human recombinant IL-2 product, which is a highly purified protein with a molecular weight of approximately 15,300 Daltons. The chemical name is desalanyl-1, serine-125 human interleukin-2. PROLEUKIN® is produced by recombinant DNA technology using a genetically engineered E. coli strain containing an analog of the human IL-2 gene encoding a modified human IL-2. Aldesleukin differs from native IL-2 in the following ways: a) Aldesleukin is not glycosylated because it is derived from E. coli; b) the molecule has no N-terminal alanine; c) the molecule has serine substituted for cysteine at amino acid position 125; and d) the aggregation state of Aldesleukin is likely to be different from that of native IL-2.

Aldesleukin amino acid sequence
SEQ ID NO: 44
MAPTSSSTKKTQLQLEHLLLDLQMILNGINNYKNP
KLTRMLTFKFYMPKKATELKHLQCLEEELKPLEEV
LNLAQSKNFHLRPRDLISNINVIVLELKGSETTFM
CEYADETATIVEFLNRWITFSQSIISTLT

In some embodiments, the IL-2 is a chemically modified human IL-2, such as a deglycosylated and/or polyethylene glycol-modified (PEGylated) IL-2. In some embodiments, the IL-2 is a PEGylated IL-2 comprising the amino acid sequence of SEQ ID NO: 44. In some embodiments, the IL-2 is a PEGylated aldesleukin. In some embodiments, the IL-2 comprises about any one of 1, 2, 3, 4, 5, 6, or more polyethylene glycol moieties. In some embodiments, the IL-2 is bempegaldesleukin.

Bempegaldesleukin (also known as NKTR-214) is an engineered IL-2R agonist with an average of six releasable polyethylene glycol (PEG) molecules attached to the IL-2Rα binding region of IL-2 (aldesleukin). This site-specific PEGylation preferentially reduces IL-2 binding to CD25 over CD122/CD132. Bempegaldesleukin and other long-acting IL-2 analogs have been described, for example, in Sharma M. et al., Nature Communications, (2020) 11:661; WO2012/065086A1, and WO2015125159A1, which are incorporated herein by reference in their entirety.

In some embodiments, the CD137-inducing agent is a histone deacetylase (HDAC) inhibitor. In some embodiments, the HDAC inhibitor is selected from the group consisting of belinostat, vorinostat, romidepsin, and chidamide. In some embodiments, the HDAC inhibitor is belinostat.

The acetylation state of histones and other proteins is maintained by histone acetyltransferase (HAT) and histone deacetylase (HDAC) enzymes. HATs catalyze the transfer of an acetyl group from acetyl-CoA to lysine residues in proteins and HDAC removes it. Depending on the mechanisms of removing the acetyl group, HDACs can be divided into two distinct families. The “classical family” comprises Zn²⁺-dependent HDACs, the second family of HDACs depends in catalysis on NAD⁺ and subsequently, O-acetyl-ADP-ribose and nicotinamide are formed as a result of the acetyl transfer. HDACs comprise a family of 18 genes in humans and are divided into four classes based on their homology to yeast analogs: class I (HDACs 1, 2, 3, 8), class IIa (HDACs 4, 5, 7, 9), class IIb (HDACs 6, 10) and class IV (HDAC11).

Various classes of HDAC inhibitors have been developed, including: 1) hydroxamic acids; 2) aliphatic acids; 3) benzamides; and 4) cyclic tetrapeptides. Vorinostat (SAHA), panobinostat (LBH-589), belinostat (PXD-101), and romidepsin (FK-228) have been approved by the US FDA for the treatment of cancer (refractory cutaneous/peripheral T-cell lymphoma).

In some embodiments, the CD137-inducing agent is a DNA-damaging agent. In some embodiments, the DNA-damaging agent is an alkylating agent. Exemplary alkylating agents include, but are not limited to, bendamustine (BENDEKA®), chlorambucil (LEUKERAN®), mcyclophosphamide (CYTOXAN®), ifosfamide (IFEX®), mechlorethamine hydrochloride (MUSTARGEN®), thiotepa (THIOPLEX®), streptozotocin (ZANOSAR®), carmustine (BICNU®, GLIADEL WAFER®), lomustine (CEENU®), and dacarbazine (DTIC-DOME®). In some embodiments, the CD137-inducing agent is bendamustine.

In some embodiments, the CD137-inducing agent is a DNA chelator. In some embodiments, the CD137-inducing agent is an alkaloid. Exemplary alkaloids include, but are not limited to, doxorubicin (ADRIAMYCIN®), epirubicin (ELLENCE®, PHARMORUBICIN®), daunorubicin (CERUBIDINE®, DAUNOXOME®), nemorubicin, idarubicin (IDAMYCIN® PFS, ZAVEDOS®), mitoxantrone (DHAD®, NOVANTRONE®), dactinomycin (actinomycin D, COSMEGEN®), plicamycin (MITHRACIN®), mitomycin (MUTAMYCIN®), and bleomycin (BLENOXANE®). In some embodiments, the CD137-inducing agent is mitomycin. In some embodiments, the CD137-inducing agent is bleomycin. In some embodiments, the CD37-inducing agent is doxorubicin. Mitomycin, bleomycin and doxorubicin have been shown to induce expression of CD137 in human T lymphocytes. See, for example, Kim K. et al., Immunology 2002, 107: 472-479.

In some embodiments, the CD137-inducing agent is a proteasome inhibitor. In some embodiments, the CD137-inducing agent is bortezomib (VELCADE®).

In some embodiments, the CD137-inducing agent is a chemotherapeutic agent. The term “chemotherapeutic agent” refers to a chemical or biological substance that can cause death of cancer cells, or interfere with growth, division, repair, and/or function of cancer cells. In some embodiments, the CD137-inducing agent is an alkaloid or a plant vinca alkaloid, such as a cytotoxic antibiotic, e.g., doxorubicin (ADRIAMYCIN®), epirubicin (ELLENCE®, PHARMORUBICIN®), daunorubicin (CERUBIDINE®, DAUNOXOME®), nemorubicin, idarubicin (IDAMYCIN® PFS, ZAVEDOS®), mitoxantrone (DHAD®, NOVANTRONE®), dactinomycin (actinomycin D, COSMEGEN®), plicamycin (MITHRACIN®), mitomycin (MUTAMYCIN®), and bleormycin (BLENOXANE®), vinorelbine tartrate (NAVELBINE®), vinblastine (VELBAN®), vincristine (ONCOVIN®), or vindesine (ELDISINE®). In some embodiments, the CD137-inducing agent is vincristine (ONCOVIN®).

### III. Anti-CD137 Antibodies

The method described herein comprise administration of an anti-CD137 antibody that specifically binds to an extracellular domain of human CD137. The anti-CD137 antibodies described herein include full-length anti-CD137 antibodies, antigen-binding fragments of the CD137 antibodies, and derivatives of the CD137 antibodies. In some embodiments, the anti-CD137 antibody is any one of the antibodies described herein, including antibodies described with reference to epitope binding and antibodies described with reference to specific amino acid sequences of CDRs, variable regions (VL, VH), and IgG (e.g., IgG1, or IgG4) light and heavy chains. In some embodiments, the anti-CD137 antibody has at least one (e.g., at least one, at least two, at least three, at least four, at least five, at least six, at least seven, eight, or all nine) of the following functional properties: (a) bind to human CD137 with a KD of 500 nM or less; (b) have agonist activity on human CD137; (c) do not bind to human OX40, CD40, GITR and/or CD27 receptor at concentration up to 1000 nM; (d) is cross-reactive with monkey, mouse, rat, or dog CD137; (e) do not induce ADCC effects; (f) are capable of inhibiting tumor cell growth; (g) have therapeutic effect on a cancer; (h) blocks binding between CD137 and CD137L; and (i) blocks CD137 signaling stimulated by CD137L (e.g., CD137L-stimulated NF-κB-dependent transcription) in a cell that expresses CD137. In some embodiments, the antibodies disclosed herein can also block, e.g., completely block, the binding between CD137 and its ligand CD137L. In some embodiments, the anti-CD137 antibody is an antibody (or an antigen-binding fragment thereof) that cross-competes for binding to human CD137 with one or more of the antibodies or antigen-binding fragments as described herein. Exemplary anti-CD137 antibodies that are suitable for the methods described herein have been described, for example, in US20190055314A1, WO2019036855A1, and WO2019037711A1, which are incorporated herein by reference in their entirety.

Human CD137, also known as tumor necrosis factor receptor superfamily member 9 (TNFRSF9), 4-1BB and induced by lymphocyte activation (ILA), is a 255 amino acid protein (e.g., GenBank Accession No. NM_001561; NP_001552; SEQ ID NO.: 1). The protein comprises a signal sequence (amino acid residues 1-17), followed by an extracellular domain (169 amino acids), a transmembrane region (27 amino acids), and an intracellular domain (42 amino acids) (Cheuk A T C et al. 2004 Cancer Gene Therapy 11: 215-226). The receptor is expressed on the cell surface in monomer and dimer forms and likely trimerizes with CD137 ligand to signal.

human CD137 amino acid sequence
SEQ ID NO: 1
MGNSCYNIVATLLLVLNFERTRSLQDPCSNCPAGT
FCDNNRNQICSPCPPNSFSSAGGQRTCDICRQCKG
VFRTRKECSSTSNAECDCTPGFHCLGAGCSMCEQD
CKQGQELTKKGCKDCCFGTFNDQKRGICRPWTNCS
LDGKSVLVNGTKERDVVCGPSPADLSPGASSVTPP
APAREPGHSPQIISFFLALTSTALLFLLFFLTLRF
SVVKRGRKKLLYIFKQPFMRPVQTTQEEDGCSCRF
PEEEEGGCEL

In some embodiments, the anti-CD137 antibody specifically binds to one or more amino acid residues within amino acid residues 34-108 of SEQ ID NO: 1. In some embodiments, the anti-CD137 antibody specifically binds to one or more amino acid residues within amino acid residues 34-93 of SEQ ID NO: 1. In some embodiments, the anti-CD137 antibody specifically binds to one or more amino acid residues selected from the group consisting of amino acid residues 34-36, 53-55, and 92-93 of SEQ ID NO:1. In some embodiments, the anti-CD137 antibody specifically binds to one or more of amino acid residues 34-36, one or more of amino acid residues 53-55, and one or more or amino acid residues 92-93 of SEQ ID NO: 1. In some embodiments, the anti-CD137 antibody does not bind to one or more of amino acid residues selected from the group consisting of amino acid residues 109-112, 125, 126, 135-138, 150 and 151 of SEQ ID NO: 1. In some embodiments, the anti-CD137 antibody specifically does not bind to amino acid residues 109-112, 125, 126, 135-138, 150 and 151 of SEQ ID NO: 1. Methods of measuring an antibody or antigen-binding fragment's ability to bind a target antigen may be carried out using any method known in the art, including for example, by surface plasmon resonance, an ELISA, isothermal titration calorimetry, a filter binding assay, an EMSA, etc., or based on the crystal structure of the anti-CD137 antibody with CD137.

In some embodiments, the anti-CD137 antibody specifically binds to one or more amino acid residues selected from the group consisting of amino acid residues 51, 53, 62-73, 83, 89, 92, 95-104 and 112-116 of SEQ ID NO: 1. In some embodiments, the anti-CD137 antibody specifically binds to one or more amino acid residues selected from the group consisting of amino acid residues 51, 53, 63-67, 69-73, 83, 89, 92, 98-104, and 112-116 of SEQ ID NO: 1. In some embodiments, the anti-CD137 antibody specifically binds to one or more amino acid residues selected from the group consisting of amino acid residues 51, 63-67, 69-73, 83, 89, 92, 98-104 and 112-114 of SEQ ID NO: 1.

In some embodiments, the anti-CD137 antibody specifically binds to amino acid residues 51, 53, 62-73, 83, 89, 92, 95-104 and 112-116 of SEQ ID NO: 1. In some embodiments, the anti-CD137 antibody specifically binds to amino acid residues 51, 53, 63-67, 69-73, 83, 89, 92, 98-104, and 112-116 of SEQ ID NO: 1. In some embodiments, the anti-CD137 antibody specifically binds to amino acid residues 51, 63-67, 69-73, 83, 89, 92, 98-104 and 112-114 of SEQ ID NO: 1.

In some embodiments, the anti-CD137 antibody specifically binds to human CD137 with a KD of about 500 nM or less (e.g., about 500 nM or less, about 400 nM or less, about 300 nM or less, about 200 nM or less, about 150 nM or less, about 100 nM or less, about 90 nM or less, about 80 nM or less, about 75 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) In some embodiments, the anti-CD137 antibody specifically binds to human CD137 with a KD of about 100 nM or less. In some embodiments, the anti-CD137 antibody specifically binds to human CD137 with a KD of about 50 nM or less. Methods of measuring the KD of an antibody or antigen-binding fragment may be carried out using any method known in the art, including for example, by surface plasmon resonance, an ELISA, isothermal titration calorimetry, a filter binding assay, an EMSA, etc.

Anti-CD137 antibodies need to be cross-linked to become agonistic. For example, cross-linking is achieved in vivo through Fcgamma receptors, while typically polyclonal anti-Fc antibodies are used in cell-based experiments in vitro. In some embodiments, the anti-CD137 antibodies described herein have agonist activity on human CD137. In some embodiments, the anti-CD137 antibody induces one or more (e.g., one or more, two or more, three or more, etc.) activities of human CD137 when a cell (e.g., a human cell) expressing human CD137 is contacted by the anti-CD137 antibody. Various CD137 activities are known in the art and may include, without limitation, induction of NF-κB-dependent transcription, induction of T cell proliferation, prolonging T cell survival, co-stimulation of activated T cells, induction of cytokine secretion (such as IL-2), and induction of monocyte activation. In some embodiments, the one or more CD137 activities is not CD137 binding to its ligand. Methods of measuring CD137 activity (e.g., the induction of NF-κB-dependent transcription and/or T cell proliferation, etc.) are known in the art. In some embodiments, the anti-CD137 antibody increases NF-κB dependent transcription in cells (e.g., human cells) expressing human CD137. In some embodiments, NF-κB dependent transcription is increased by about 10% or more, about 20% or more, about 30% or more, about 40% or more, about 50% or more, about 60% or more, about 70% or more, about 80% or more, about 90% or more, or about 99% or more in cells (e.g., human cells) expressing CD137 contacted with the anti-CD137 antibody, relative to a corresponding cell not contacted with the anti-CD137 antibody (e.g., a corresponding cell not contacted with an antibody, or contacted with an isotype control antibody). In some embodiments, NF-κB dependent transcription is increased by about 2-fold, 3-fold, 4-fold, 5-fold, 6-fold, 7-fold, 8-fold, 9-fold, 10-fold, 100-fold, 1000-fold or more in cells (e.g., human cells) expressing CD137 contacted with the anti-CD137 antibody, relative to a corresponding cell not contacted with the anti-CD137 antibody (e.g., a corresponding cell not contacted with an antibody, or contacted with an isotype control antibody).

In some embodiments, the anti-CD137 antibody is cross-reactive with monkey (e.g., cynomolgus monkey), mouse, rat, and/or dog CD137. In some embodiments, the anti-CD137 antibody is cross-reactive with monkey CD137. In some embodiments, the anti-CD137 antibody is cross-reactive with mouse CD137. In some embodiments, the anti-CD137 antibody is cross-reactive with rat CD137. In some embodiments, the anti-CD137 antibody is cross-reactive with dog CD137. In some embodiments, the anti-CD137 antibody is cross-reactive with monkey and mouse CD137; monkey and rat CD137; monkey and dog CD137; mouse and rat CD137; mouse and dog CD137; rat and dog CD137; monkey, mouse, and rat CD137; monkey, mouse, and dog CD137; monkey, rat, and dog CD137; mouse, rat, and dog CD137; or monkey, mouse, rat, and dog CD137. In some embodiments, the anti-CD137 antibody is cross-reactive at about 100 nM (e.g., at about 1 nM, at about 10 nM, at about 25 nM, at about 50 nM, at about 75 nM, at about 100 nM). Methods of measuring antibody cross-reactivity are known in the art, including, without limitation, surface plasmon resonance, an ELISA, isothermal titration calorimetry, a filter binding assay, an EMSA, etc.

In some embodiments, the anti-CD137 antibody does not induce ADCC effects. Methods of measuring ADCC effects (e.g., in vivo methods) are known in the art. In some embodiments, the anti-CD137 antibody does not ADCC effects by more than about 10% (do not induce ADCC by more than about 10%, more than about 5%, more than about 1%, more than about 0.1%, more than about 0.01%) relative to a control.

In some embodiments, the anti-CD137 antibody is capable of inhibiting tumor cell growth/proliferation. In some embodiments, the tumor cell growth/proliferation is inhibited by at least about 5% (e.g., at least about 5%, at least about 10%, at least about 20%, at least about 30%, at least about 40%, at least about 50%, at least about 60%, at least about 70%, at least about 800%, at least about 90%, or at least about 99%) when contacted with the anti-CD137 antibody relative to corresponding tumor cells not contacted with the anti-CD137 antibody. In some embodiments, the anti-CD137 antibody is capable of reducing tumor volume in a subject when the subject is administered the anti-CD137 antibody. In some embodiments, the anti-CD137 antibody is capable of reducing tumor volume in a subject by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) relative to the initial tumor volume in the subject (e.g., prior to administration of the anti-CD137 antibody). Methods of monitoring tumor cell growth/proliferation, tumor volume, and/or tumor inhibition are known in the art.

In some embodiments, the anti-CD137 antibody has therapeutic effect on a cancer. In some embodiments, the anti-CD137 antibody reduces one or more signs or symptoms of a cancer. In some embodiments, a subject suffering from a cancer goes into partial or complete remission when administered the anti-CD137 antibody.

In some embodiments, the anti-CD137 antibody is selected from the group consisting of AG10058, AG10059 and ADG106. In some embodiments, the anti-CD137 antibody competes or cross-competes for binding to human CD137 with any of the illustrative antibodies of the present application, such as AG10058, AG10059 and ADG106. In some embodiments, the anti-CD137 antibody is an antibody that competes or cross-competes for binding to the same epitope on human CD137 as AG10058, AG10059 or ADG106. The ability of an antibody to compete or cross-compete for binding with another antibody can be determined using standard binding assays known in the art, such as BIAcore analysis, ELISA assays, or flow cytometry. For example, one can allow an illustrative antibody of the disclosure to bind to human CD137 under saturating conditions and then measure the ability of the test antibody to bind to the CD137. If the test antibody is able to bind to the CD137 at the same time as the illustrative antibody, then the test antibody binds to a different epitope as the illustrative antibody. However, if the test antibody is not able to bind to the CD137 at the same time, then the test antibody binds to the same epitope, an overlapping epitope, or an epitope that is in close proximity to the epitope bound by the illustrative antibody. This experiment can be performed using various methods, such as ELISA, RIA, FACS or surface plasmon resonance.

In some embodiments, the anti-CD137 antibody blocks the binding between CD137 and its ligand (e.g., human CD137 and human CD137L). In some embodiments, the anti-CD137 antibody blocks the binding between CD137 and its ligand in vitro. In some embodiments, the anti-CD137 antibody has a half maximal inhibitory concentration (IC50) of about 500 nM or less (e.g., about 500 nM or less, about 400 nM or less, about 300 nM or less, about 200 nM or less, about 100 nM or less, about 50 nM or less, about 25 nM or less, about 10 nM or less, about 1 nM or less, etc.) for blocking binding of CD137 its ligand. In some embodiments, the anti-CD137 antibody has a half-maximal inhibitory concentration (IC50) of about 100 nM or less for blocking binding of CD137 its ligand. In some embodiments, the anti-CD137 antibody completely blocks binding of human CD137 to its ligand when provided at a concentration of about 100 nM or greater (e.g., about 100 nM or greater, about 500 nM or greater, about 1 μM or greater, about 10 μM or greater, etc.). As used herein, the term “complete blocking” or “completely blocks” refers to the antibody or antigen-binding fragment's ability to reduce binding between a first protein and a second protein by at least about 80% (e.g., at least about 80%, at least about 85%, at least about 90%, at least about 95%, at least about 99%, etc.). Methods of measuring the ability of an antibody or antigen-binding fragment to block binding of a first protein (e.g., a CD137) and a second protein (e.g., CD137L) are known in the art, including, without limitation, via BIAcore analysis, ELISA assays, and flow cytometry.

In some embodiments, the anti-CD137 antibody comprises a heavy chain variable region (VH) and a light chain variable region (VL), a) wherein the VH comprises a HVR-H1, a HVR-H2, and aHVR-H3, wherein the HVR-H1 comprises an amino acid sequence according to a formula selected from the group consisting of: Formula (I): XITFX2X3YX4IHWV (SEQ ID NO: 32), wherein X1 is F or Y, X2 is S or T, X3 is G, N, or S, and X4 is A, G, or W; Formula (II): YSIX ISGX2X3WX4WI (SEQ ID NO: 33), wherein X1 is S or T, X2 is H or Y, X3 is H or Y, and X4 is A, D, G, N, S, or T; and Formula (III): FSLSTX1GVX2VX3WI (SEQ ID NO: 34), wherein X1 is G or S, X2 is A or G, and X3 is A, G, S, or T; wherein the HVR-H2 comprises an amino acid sequence according to a formula selected from the group consisting of: Formula (IV): LALIDWX1X2DKX3YSX4SLKSRL (SEQ ID NO: 35), wherein X1 is A, D, or Y, X2 is D or G, X3 is R, S, or Y, and X4 is P or T; Formula (V): IGX1IYHSGX2TYYX3PSLKSRV (SEQ ID NO: 36), wherein X1 is D or E, X2 is N or S, and X3 is N or S; and Formula (VI): VSX1ISGX2GX3X4TYYADSVKGRF (SEQ ID NO: 37), wherein X1 is A, G, S, V, or Y, X2 is A, D, S, or Y, X3 is D, G, or S, and X4 is S or T; and wherein the HVR-H3 comprises an amino acid sequence according to Formula (VII): ARX1GX2X3X4VX5GDWFX6Y (SEQ ID NO: 38), wherein X1 is E or G, X2 is E or S, X3 is D or T, X4 is A, T, or V, X5 is A, I, L, T, or V, and X6 is A, D, or G; and/or b) wherein the VL comprises a HVR-L1, a HVR-L2, and a HVR-L3, wherein the HVR-L1 comprises an amino acid sequence according to Formula (VIII): X1ASQX2X3X4X5X6X7X8 (SEQ ID NO: 39), wherein X1 is Q or R, X2 is D, G, or S, X3 is I or V, X4 is G, R, S, or T, X5 is P, R, S, or T, X6 is A, D, F, S, V, or Y, X7 is L or V, and X8 is A, G, or N; wherein the HVR-L2 comprises an amino acid sequence according to Formula (IX): X1ASX2X3X4X5GX6 (SEQ ID NO: 40), wherein X1 is A or D, X2 is N, S, or T, X3 is L or R, X4 is A, E, or Q, X5 is S or T, and X6 is I or V; and wherein the HVR-L3 comprises an amino acid sequence according to a formula selected from the group consisting of: Formula (X): YCQQX1YX2X3X4T (SEQ ID NO: 41), wherein X1 is A, G, S, or Y, X2 is Q, S, or Y, X3 is I, L, T, or Y, and X4 is I, S, V, or W; and Formula (XI): YCX1QX2X3X4X5PX6T (SEQ ID NO: 42), wherein X1 is E or Q, X2 is P, S, or Y, X3 is D, L, S, T, or Y, X4 is D, E, H, S, or T, X5 is D, L T, or W, and X6 is L, P, R, or V.

In some embodiments, the anti-CD137 antibody comprises a VH and a VL, wherein the VH comprises a HVR-H1 comprising the amino acid sequence of SEQ ID NO: 34, a HVR-H2 comprising the amino acid sequence of SEQ ID NO: 35, and a HVR-H3 comprising the amino acid sequence of SEQ ID NO: 38; and/or wherein the VL comprises a HVR-L1 comprising the amino acid sequence of SEQ ID NO: 39, a HVR-L2 comprising the amino acid sequence of SEQ ID NO: 40, and a HVR-L3 comprising the amino acid sequence of SEQ ID NO: 41.

Sequences of exemplary anti-CD137 antibodies are shown in Table B below.

TABLE B
Exemplary anti-CD antibodies

| Antibody | HVR-H1 | HVR-H2 | HVR-H3 | HVR-L1 | HVR-L2 | HVR-L3 |
|---|---|---|---|---|---|---|
| ADG106 | FSLSTGG | LALIDWA | ARGGSDT | RASQSIGS | DASNLET | YCQQGY |
| | VGVGWI | DDKYYSP | VIGDWFA | YLA (SEQ | GV (SEQ | YLWT |
| | (SEQ ID | SLKSRL | Y (SEQ ID | ID NO: 5) | ID NO: 6) | (SEQ ID |
| | NO: 2) | (SEQ ID | NO: 4) | | | NO. 7) |
| | | NO: 3) | | | | |
| AG10059 | YSITSGHY | VSSISGYG | ARGGSDA | RASQGIG | DASNLET | YCQQGY |
| | WAWI | STTYYAD | VLGDWF | SFLA (SEQ | GV (SEQ | YLWT |
| | (SEQ ID | SVKGRF | AY (SEQ | ID NO: 15) | ID NO: 16) | (SEQ ID |
| | NO: 12) | (SEQ ID | ID NO: 14) | | | NO: 17) |
| | | NO: 13) | | | | |
| AG10058 | FSLSTSGV | LALIDWD | ARGGSDT | RASQSVS | DASSLES | YCQQGYS |
| | GVGWI | DDKYYSP | VLGDWF | PYLA | GV (SEQ | LWT (SEQ |
| | (SEQ ID | SLKSRL | AY (SEQ | (SEQ ID | ID NO: 26) | ID NO: 27) |
| | NO: 22) | (SEQ ID | ID NO: 24) | NO: 25) | | |
| | | NO: 23) | | | | |

In some embodiments, the anti-CD137 antibody comprises a VH and a VL, wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4; and/or wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7.

In some embodiments, the anti-CD137 antibody comprises a VH comprising a heavy chain complementarity determining region (HC-CDR) 1, a HC-CDR2, and a HC-CDR3 of the amino acid sequence of SEQ ID NO: 8; and/or a VL comprising a light chain complementarity determining region (LC-CDR) 1, a LC-CDR2, and a LC-CDR3 of the amino acid sequence of SEQ ID NO: 9. In certain embodiments, the anti-CD137 antibody comprises a heavy chain variable region comprising the amino acid sequence of SEQ ID NO: 8, and/or a light chain variable region comprising the amino acid sequence of SEQ ID NO: 9. In certain embodiments, the anti-CD137 antibody comprises a heavy chain comprising the amino acid sequence of SEQ ID NO: 10, and/or a light chain comprising the amino acid sequence of SEQ ID NO: 11.

ADG106 VH
SEQ ID NO: 8
EVQLVESGGGLVQPGGSLRLSCAASGFSLSTGGVG
VGWIRQAPGKGLEWLALIDWADDKYYSPSLKSRLT
ISRDNSKNTLYLQLNSLRAEDTAVYYCARGGSDTV
IGDWFAYWGQGTLVTVSS
ADG106 VL
SEQ ID NO: 9
DIQLTQSPSSLSASVGDRVTITCRASQSIGSYLAW
YQQKPGKAPKLLIYDASNLETGVPSRFSGSGSGTD
FTLTISSLQPEDFATYYCQQGYYLWTFGQGTKVEI
KR
ADG106 Heavy Chain
SEQ ID NO: 10
EVQLVESGGGLVQPGGSLRLSCAASGFSLSTGGVG
VGWIRQAPGKGLEWLALIDWADDKYYSPSLKSRLT
ISRDNSKNTLYLQLNSLRAEDTAVYYCARGGSDTV
IGDWFAYWGQGTLVTVSSASTKGPSVFPLAPCSRS
TSESTAALGCLVKDYFPEPVTVSWNSGALTSGVHT
FPAVLQSSGLYSLSSVVTVPSSSLGTKTYTCNVDH
KPSNTKVDKRVESKYGPPCPPCPAPEFLGGPSVFL
FPPKPKDTLMISRTPEVTCVVVDVSQEDPEVQFNW
YVDGVEVHNAKTKPREEQFNSTYRVVSVLTVLHQD
WLNGKEYKCKVSNKGLPSSIEKTISKAKGQPREPQ
VYTLPPSQEEMTKNQVSLTCLVKGFYPSDIAVEWE
SNGQPENNYKTTPPVLDSDGSFFLYSRLTVDKSRW
QEGNVFSCSVMHEALHNHYTQKSLSLSLGK
ADG106 Lieht Chain
SEQ ID NO: 11
DIQLTQSPSSLSASVGDRVTITCRASQSIGSYLAW
YQQKPGKAPKLLIYDASNLETGVPSRFSGSGSGTD
FTLTISSLQPEDFATYYCQQGYYLWTFGQGTKVEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC

In some embodiments, the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14; and/or wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 17.

In some embodiments, the anti-CD137 antibody comprises a VH comprising a HC-CDR1, a HC-CDR2, and a HC-CDR3 of the amino acid sequence of SEQ ID NO: 18; and/or a VL comprising a LC-CDR1, a LC-CDR2, and a LC-CDR3 of the amino acid sequence of SEQ ID NO: 19. In certain embodiments, the anti-CD137 antibody comprises a heavy chain variable region comprising the amino acid sequence of SEQ ID NO: 18, and/or a light chain variable region comprising the amino acid sequence of SEQ ID NO: 19. In certain embodiments, the anti-CD137 antibody comprises a heavy chain comprising the amino acid sequence of SEQ ID NO: 20, and/or a light chain comprising the amino acid sequence of SEQ ID NO: 21.

ADG10059 VH
SEQ ID NO: 18
EVQLVESGGGLVQPGGSLRLSCAASGYSITSGHYW
AWIRQAPGKGLEWVSSLSGYGSTTYYADSVKGRFT
ISRDNSKNTLYLQLNSLRAEDTAVYYCARGGSDAV
LGDWFAYWGQGTLVTVSS
ADG10059 VL
SEQ ID NO: 19
DIQLTQSPSSLSASVGDRVTITCRASQGIGSFLAW
YQQKPGKAPKLLIYDASNLETGVPSRFSGSGSGTD
FTLTISSLQPEDFATYYCQQGYYLWTFGQGTKVEI
KR
ADG10059 Heavy chain
SEQ ID NO. 20
EVQLVESGGGLVQPGGSLRLSCAASGYSITSGHYW
AWIRQAPGKGLEWVSSISGYGSTTYYADSVKGRFT
ISRDNSKNTLYLQLNSLRAEDTAVYYCARGGSDAV
LGDWFAYWGQGTLVTVSSASTKGPSVFPLAPCSRS
TSESTAALGCLVKDYFPEPVTVSWNSGALTSGVHT
FPAVLQSSGLYSLSSVVTVPSSSLGTKTYTCNVDH
KPSNTKVDKRVESKYGPPCPPCPAPEFLGGPSVFL
FPPKPKDTLMISRTPEVTCVVVDVSQEDPEVQFNW
YVDGVEVHNAKTKPREEQFNSTYRVVSVLTVLHQD
WLNGKEYKCKVSNKGLPSSIEKTISKAKGQPREPQ
VYTLPPSQEEMTKNQVSLTCLVKGFYPSDIAVEWE
SNGQPENNYKTTPPVLDSDGSFFLYSRLTVDKSRW
QEGNVFSCSVMHEALHNHYTQKSLSLSLGK
ADG10059 Light chain
SEQ ID NO: 21
DIQLTQSPSSLSASVGDRVTITCRASQGIGSFLAW
YQQKPGKAPKLLIYDASNLETGVPSRFSGSGSGTD
FTLTISSLQPEDFATYYCQQGYYLWTFGQGTKVEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GE

In some embodiments, the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24; and/or wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27.

In some embodiments, the anti-CD137 antibody comprises a VH comprising a HC-CDR1, aHC-CDR2, and a HC-CDR3 of the amino acid sequence of SEQ ID NO: 28; and/or a VL comprising a LC-CDR1, a LC-CDR2, and a LC-CDR3 of the amino acid sequence of SEQ ID NO: 29. In certain embodiments, the anti-CD137 antibody comprises heavy chain variable region comprising the amino acid sequence of SEQ ID NO: 28, and/or a light chain variable region comprising the amino acid sequence of SEQ ID NO: 29. In certain embodiments, the anti-CD137 antibody comprises a heavy chain comprising the amino acid sequence of SEQ ID NO: 30, and/or a light chain comprising the amino acid sequence of SEQ ID NO: 31.

AG 10058 VH
SEQ ID NO: 28
EVQLVESGGGLVQPGGSLRLSCAASGFSLSTSGVG
VGWIRQAPGKGLEWLALIDWDDDKYYSPSLKSRLT
ISRDNSKNTLYLQLNSLRAEDTAVYYCARGGSDTV
LGDWFAYWGQGTLVTVSS
AG10058 VL
SEQ ID NO: 29
DIQLTQSPSSLSASVGDRVTITCRASQSVSPYLAW
YQQKPGKAPKLLIYDASSLESGVPSRFSGSGSGTD
FTLTISSLQPEDFATYYCQQGYSLWTFGQGTKVEI
KR
AG 10058 Heavy chain
SEQ ID NO: 30
EVQLVESGGGLVQPGGSLRLSCAASGFSLSTSGVG
VGWIRQAPGKGLEWLALIDWDDDKYYSPSLKSRLT
ISRDNSKNTLYLQLNSLRAEDTAVYYCARGGSDTV
LGDWFAYWGQGTLVTVSSASTKGPSVFPLAPCSRS
TSESTAALGCLVKDYFPEPVTVSWNSGALTSGVHT
FPAVLQSSGLYSLSSVVTVPSSSLGTKTYTCNVDH
KPSNTKVDKRVESKYGPPCPPCPAPEFLGGPSVFL
FPPKPKDTLMISRTPEVTCVVVDVSQEDPEVQFNW
YVDGVEVHNAKTKPREEQFNSTYRVVSVLTVLHQD
WLNGKEYKCKVSNKGLPSSIEKTISKAKGQPREPQ
VYTLPPSQEEMTKNQVSLTCLVKGFYPSDIAVEWE
SNGQPENNYKTTPPVLDSDGSFFLYSRLTVDKSRW
QEGNVFSCSVMHEALHNHYTQKSLSLSLGK
AG10058 Light chain
SEQ ID NO: 31
DIQLTQSPSSLSASVGDRVTITCRASQSVSPYLAW
YQQKPGKAPKLLIYDASSLESGVPSRFSGSGSGTD
FTLTISSLQPEDFATYYCQQGYSLWTFGQGTKVEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC

The CD137 antibodies described herein can be in any class, such as IgG, IgM, IgE, IgA, or IgD. It is preferred that the CD137 antibodies are in the IgG class, such as IgG1, IgG2, IgG3, or IgG4 subclass. A CD137 antibody can be converted from one class or subclass to another class or subclass using methods known in the art. An exemplary method for producing an antibody in a desired class or subclass comprises the steps of isolating a nucleic acid encoding a heavy chain of an CD137 antibody and a nucleic acid encoding a light chain of a CD137 antibody, isolating the sequence encoding the VH region, ligating the VH sequence to a sequence encoding a heavy chain constant region of the desired class or subclass, expressing the light chain gene and the heavy chain construct in a cell, and collecting the CD137 antibody. In some embodiments, the anti-CD137 antibody comprises a human IgG1 Fc region. In some embodiments, the anti-CD137 antibody comprises a human IgG4 Fc region. In some embodiments, the human IgG4 Fc region comprises an S241P mutation, wherein numbering is according to Kabat. In some embodiments, the anti-CD137 antibody comprises an Fc region comprising one or more mutations that promotes cross-linking.

### Antigen-Binding Fragments and Antibody Derivatives

In some embodiments, the anti-CD137 antibody is an antigen-binding fragment of any one of the anti-CD137 antibodies described herein.

In some embodiments, the antigen-binding fragments of an CD137 antibody include: (i) a Fab fragment, which is a monovalent fragment consisting of the V_{L}, V_{H}, C_{L} and C_{H}1 domains; (ii) a F(ab′)₂ fragment, which is a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al., (1989) Nature 341:544-546), which consists of a V_{H} domain; (vi) an isolated CDR, and (vii) single chain antibody (scFv), which is a polypeptide comprising a V_{L} region of an antibody linked to a V_{H} region of an antibody. Bird et al., (1988) Science 242:423-426 and Huston et al., (1988) Proc. Natl. Acad. Sci. USA 85:5879-5883.

In some embodiments, the anti-CD137 antibody is a derivative of any one of the anti-CD137 antibodies described herein.

In some embodiments, the antibody derivative is derived from modifications of the amino acid sequences of an illustrative antibody (“parent antibody”) of the disclosure while conserving the overall molecular structure of the parent antibody amino acid sequence. Amino acid sequences of any regions of the parent antibody chains may be modified, such as framework regions, CDR regions, or constant regions. Types of modifications include substitutions, insertions, deletions, or combinations thereof, of one or more amino acids of the parent antibody.

In some embodiments, the antibody derivative comprises a VH comprising an amino acid sequence that is at least 80%, at least 85%, at least 90%, at least 95%, at least 96%, at least 97%, at least 98%, or at least 99% identical to the amino acid sequence of SEQ ID NO: 8; and/or a VL comprising an amino acid sequence that is at least 80%, at least 85%, at least 90%, at least 95%, at least 96%, at least 97%, at least 98%, or at least 99% identical to the amino acid sequence of SEQ ID NO: 9. In some embodiments, the antibody derivative comprises a HVR-H1 amino acid sequence region that is at least 80%, at least 85%, at least 90%, at least 95%, at least 96%, at least 97%, at least 98%, or at least 99% identical to an amino acid sequence as set forth in SEQ ID NO: 2. In some embodiments, the antibody derivative comprises a HVR-H2 amino acid sequence region that is at least 80%, at least 90%, at least 95%, at least 96%, at least 97%, at least 98%, or at least 99% identical to an amino acid sequence as set forth in SEQ ID NO: 3. In some embodiments, the antibody derivative comprises a HVR-H3 amino acid sequence region that is at least 80%, at least 85%, at least 90%, at least 95%, at least 96%, at least 97%, at least 98%, or at least 99% identical to an amino acid sequence as set forth in SEQ ID NO: 4. In some embodiments, the antibody derivative comprises a HVR-L1 amino acid sequence region that is at least 80%, at least 85%, at least 90%, at least 95%, at least 96%, at least 97%, at least 98%, or at least 99% identical to an amino acid sequence as set forth in SEQ ID NO: 5. In some embodiments, the antibody derivative comprises a HVR-L2 amino acid sequence region that is at least 80%, at least 90%, at least 95%, at least %%, at least 97%, at least 98%, or at least 99% identical to an amino acid sequence asset forth in SEQ ID NO: 6. In some embodiments, the antibody derivative comprises a HVR-L3 amino acid sequence region that is at least 80%, at least 85%, at least 90%, at least 95%, at least 96%, at least 97%, at least 98%, or at least 99% identical to an amino acid sequence as set forth in SEQ ID NO: 7. In some particular embodiments, the antibody derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence as set forth in any of SEQ ID NOs: 8, 9, 10, and 11.

In some embodiments, the antibody derivative comprises a VH comprising an amino acid sequence that is at least 80%, at least 85%, at least 90%, at least 95%, at least 96%, at least 97%, at least 98%, or at least 99% identical to the amino acid sequence of SEQ ID NO: 18; and/or a VL comprising an amino acid sequence that is at least 80%, at least 85%, at least 90%, at least 95%, at least 96%, at least 97%, at least 98%, or at least 99% identical to the amino acid sequence of SEQ ID NO: 19. In some embodiments, the antibody derivative comprises a HVR-H1 amino acid sequence region that is at least 80%, at least 85%, at least 90%, at least 95%, at least 96%, at least 97%, at least 98%, or at least 99% identical to an amino acid sequence as set forth in SEQ ID NO: 12. In some embodiments, the antibody derivative comprises a HVR-H2 amino acid sequence region that is at least 80%, at least 90%, at least 95%, at least 96%, at least 97%, at least 98%, or at least 99% identical to an amino acid sequence as set forth in SEQ ID NO: 13. In some embodiments, the antibody derivative comprises a HVR-H3 amino acid sequence region that is at least 80%, at least 85%, at least 90%, at least 95%, at least 96%, at least 97%, at least 98%, or at least 99% identical to an amino acid sequence as set forth in SEQ ID NO: 14. In some embodiments, the antibody derivative comprises a HVR-L1 amino acid sequence region that is at least 80%, at least 85%, at least 90%, at least 95%, at least 96%, at least 97%, at least 98%, or at least 99% identical to an amino acid sequence as set forth in SEQ ID NO: 15. In some embodiments, the antibody derivative comprises a HVR-L2 amino acid sequence region that is at least 80%, at least 90%, at least 95%, at least 96%, at least 97%, at least 98%, or at least 99% identical to an amino acid sequence as set forth in SEQ ID NO. 16. In some embodiments, the antibody derivative comprises a HVR-L3 amino acid sequence region that is at least 80%, at least 85%, at least 90%, at least 95%, at least 96%, at least 97%, at least 98%, or at least 99% identical to an amino acid sequence as set forth in SEQ ID NO: 17. In some particular embodiments, the antibody derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence as set forth in any of SEQ ID NOs: 18, 19, 20, and 21.

In some embodiments, the antibody derivative comprises a VH comprising an amino acid sequence that is at least 80%, at least 85%, at least 90%, at least 95%, at least 96%, at least 97%, at least 98%, or at least 99% identical to the amino acid sequence of SEQ ID NO: 28; and/or a VL comprising an amino acid sequence that is at least 80%, at least 85%, at least 90%, at least 95%, at least 96%, at least 97%, at least 98%, or at least 99% identical to the amino acid sequence of SEQ ID NO: 29. In some embodiments, the antibody derivative comprises a HVR-H1 amino acid sequence region that is at least 80%, at least 85%, at least 90%, at least 95%, at least 96%, at least 97%, at least 98%, or at least 99% identical to an amino acid sequence as set forth in SEQ ID NO: 22. In some embodiments, the antibody derivative comprises a HVR-H2 amino acid sequence region that is at least 80%, at least 90%, at least 95%, at least 96%, at least 97%, at least 98%, or at least 99% identical to an amino acid sequence as set forth in SEQ ID NO: 23. In some embodiments, the antibody derivative comprises a HVR-H3 amino acid sequence region that is at least 80%, at least 85%, at least 90%, at least 95%, at least 96%, at least 97%, at least 98%, or at least 99% identical to an amino acid sequence as set forth in SEQ ID NO: 24. In some embodiments, the antibody derivative comprises a HVR-L1 amino acid sequence region that is at least 80%, at least 85%, at least 90%, at least 95%, at least 96%, at least 97%, at least 98%, or at least 99% identical to an amino acid sequence as set forth in SEQ ID NO: 25. In some embodiments, the antibody derivative comprises a HVR-L2 amino acid sequence region that is at least 80%, at least 90%, at least 95%, at least 96%, at least 97%, at least 98%, or at least 99% identical to an amino acid sequence as set forth in SEQ ID NO: 26. In some embodiments, the antibody derivative comprises a HVR-L3 amino acid sequence region that is at least 80%, at least 85%, at least 90%, at least 95%, at least 96%, at least 97%, at least 98%, or at least 99% identical to an amino acid sequence as set forth in SEQ ID NO: 27. In some particular embodiments, the antibody derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence as set forth in any of SEQ ID NOs: 28, 29, 30, and 31.

Amino acid substitutions encompass both conservative substitutions and non-conservative substitutions. The term “conservative amino acid substitution” means a replacement of one amino acid with another amino acid where the two amino acids have similarity in certain physico-chemical properties such as polarity, charge, solubility, hydrophobicity, hydrophilicity, and/or the amphipathic nature of the residues involved. For example, substitutions typically may be made within each of the following groups: (a) nonpolar (hydrophobic) amino acids, such as alanine, leucine, isoleucine, valine, proline, phenylalanine, tryptophan, and methionine; (b) polar neutral amino acids, such as glycine, serine, threonine, cysteine, tyrosine, asparagine, and glutamine; (c) positively charged (basic) amino acids, such as arginine, lysine, and histidine; and (d) negatively charged (acidic) amino acids, such as aspartic acid and glutamic acid.

The modifications may be made in any positions of the amino acid sequences of the antibody, including the CDRs, framework regions, or constant regions. In some embodiments, the present disclosure provides an antibody derivative that contains the VH and VL CDR sequences of an illustrative antibody of this disclosure, yet contains framework sequences different from those of the illustrative antibody. Such framework sequences can be obtained from public DNA databases or published references that include germline antibody gene sequences. For example, germline DNA sequences for human heavy and light chain variable region genes can be found in the Genbank database or in the “VBase” human germline sequence database (Kabat, E. A., et al., Sequences of Proteins of Immunological Interest, Fifth Edition, U.S. Department of Health and Human Services, NIH Publication No. 91-3242 (1991): Tomlinson. I. M., et al., J. Mol. Biol. 227:776-798 (1992); and Cox, J. P. L. et al., Eur. J. Immunol. 24:827-836 (1994)). Framework sequences that may be used in constructing an antibody derivative include those that are structurally similar to the framework sequences used by illustrative antibodies of the disclosure, e.g., similar to the VH 3-23 framework sequences and/or the VL λ3 or λ1-13 framework sequences used by illustrative antibodies of the disclosure. For example, the HVR-H1, HVR-H2, and HVR-H3 sequences, and the HVR-L1, HVR-L2, and HVR-L3 sequences of an illustrative antibody can be grafted onto framework regions that have the identical sequence as that found in the germline immunoglobulin gene from which the framework sequence derive, or the CDR sequences can be grafted onto framework regions that contain one or more mutations as compared to the germline sequences.

In some embodiments, the antibody derivative is a chimeric antibody, which comprises an amino acid sequence of an illustrative antibody of the disclosure. In one example, one or more CDRs from one or more illustrative human antibodies are combined with CDRs from an antibody from a non-human animal, such as mouse or rat. In another example, all of the CDRs of the chimeric antibody are derived from one or more illustrative antibodies. In some particular embodiments, the chimeric antibody comprises one, two, or three CDRs from the heavy chain variable region or from the light chain variable region of an illustrative antibody. Chimeric antibodies can be generated using conventional methods known in the art.

Another type of modification is to mutate amino acid residues within the CDR regions of the VH and/or VL chain. Site-directed mutagenesis or PCR-mediated mutagenesis can be performed to introduce the mutation(s) and the effect on antibody binding, or other functional property of interest, can be evaluated in in vitro or in vivo assays known in the art. Typically, conservative substitutions are introduced. The mutations may be amino acid additions and/or deletions. Moreover, typically no more than one, two, three, four or five residues within a CDR region are altered. In some embodiments, the antibody derivative comprises 1, 2, 3, or 4 amino acid substitutions in the heavy chain CDRs and/or in the light chain CDRs. In some embodiments, the amino acid substitution is to change one or more cysteines in an antibody to another residue, such as, without limitation, alanine or serine. The cysteine may be a canonical or non-canonical cysteine. In some embodiments, the antibody derivative has 1, 2, 3, or 4 conservative amino acid substitutions in the heavy chain CDR regions relative to the amino acid sequences of an illustrative antibody.

Modifications may also be made to the framework residues within the VH and/or VL regions. Typically, such framework variants are made to decrease the immunogenicity of the antibody. One approach is to “back mutate” one or more framework residues to the corresponding germline sequence. An antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis.

In addition, modifications may also be made within the Fc region of an illustrative antibody, typically to alter one or more functional properties of the antibody, such as serum half-life, complement fixation, Fc receptor binding, and/or antigen-dependent cellular cytotoxicity. In one example, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody. In another case, the Fc hinge region of an antibody is mutated to decrease the biological half-life of the antibody.

Furthermore, an antibody of the disclosure may be modified to alter its potential glycosylation site or pattern in accordance with routine experimentation known in the art. In some embodiments, the anti-CD137 antibody derivative contains at least one mutation in a variable region of a light chain or heavy chain that changes the pattern of glycosylation in the variable region. Such an antibody derivative may have an increased affinity and/or a modified specificity for binding an antigen. The mutations may add a novel glycosylation site in the V region, change the location of one or more V region glycosylation site(s), or remove a pre-existing V region glycosylation site. In some embodiments, the anti-CD137 antibody derivative has a potential N-linked glycosylation site at asparagine in the heavy chain variable region, wherein the potential N-linked glycosylation site in one heavy chain variable region is removed. In some embodiments, the anti-CD137 antibody derivative has having a potential N-linked glycosylation site at asparagine in the heavy chain variable region, wherein the potential N-linked glycosylation site in both heavy chain variable regions is removed. Method of altering the glycosylation pattern of an antibody is known in the art, such as those described in U.S. Pat. No. 6,933,368, the disclosure of which incorporated herein by reference.

In some embodiments, the antibody derivative is a CD137 antibody multimer, which is a multimeric form of a CD137 antibody, such as antibody dimers, trimers, or higher-order multimers of monomeric antibodies. Individual monomers within an antibody multimer may be identical or different. In addition, individual antibodies within a multimer may have the same or different binding specificities. Multimerization of antibodies may be accomplished through natural aggregation of antibodies. For example, some percentage of purified antibody preparations (e.g., purified IgG1 or IgG4 molecules) spontaneously form protein aggregates containing antibody homodimers, and other higher-order antibody multimers. Alternatively, antibody homodimers may be formed through chemical linkage techniques known in the art, such as through using crosslinking agents. Suitable crosslinkers include those that are heterobifunctional, having two distinctly reactive groups separated by an appropriate spacer (such as m-maleimidobenzoyl-N-hydroxysuccinimide ester, succinimidyl 4-(maleimidomethyl)cyclohexane-1-carboxylate, and N-succinimidyl S-acethylthio-acetate) or homobifunctional (such as disuccinimidyl suberate). Such linkers are commercially available from, for example, Pierce Chemical Company, Rockford, Ill. Antibodies can also be made to multimerize through recombinant DNA techniques known in the art.

In some embodiments, the anti-CD137 antibody is a multimeric antibody (e.g., a bispecific antibody). In some embodiments, the anti-CD137 antibody is an IgM antibody, e.g., comprises an IgM Fc region (e.g., a human IgM Fc region).

Examples of other antibody derivatives provided by the present disclosure include single chain antibodies, diabodies, domain antibodies, and unibodies. A “single-chain antibody” (scFv) consists of a single polypeptide chain comprising a VL domain linked to a VH domain wherein VL domain and VH domain are paired to form a monovalent molecule. Single chain antibody can be prepared according to method known in the art (see, for example, Bird et al., (1988) Science 242:423-426 and Huston et al., (1988) Proc. Natl. Acad. Sci. USA 85:5879-5883). A “diabody” consists of two chains, each chain comprising a heavy chain variable region connected to a light chain variable region on the same polypeptide chain connected by a short peptide linker, wherein the two regions on the same chain do not pair with each other but with complementary domains on the other chain to form a bispecific molecule. Methods of preparing diabodies are known in the art (See, e.g., Holliger P. et al., (1993) Proc. Natl. Acad. Sci. USA 90:6444-6448, and Poljak R. J. et al., (1994) Structure 2:1121-1123). Domain antibodies (dAbs) are small functional binding units of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821. WO04/003019 and WO03/002609). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### Methods of Making

Antibodies of the present disclosure can be produced by techniques known in the art, including conventional monoclonal antibody methodology e.g., the standard somatic cell hybridization technique (See e.g., Kohler and Milstein, Nature 256:495 (1975), viral or oncogenic transformation of B lymphocytes, or recombinant antibody technologies as described in detail herein below.

Hybridoma production is a very well established procedure. The common animal system for preparing hybridomas is the murine system. Immunization protocols and techniques for isolation of immunized splenocytes for fusion are known in the art. Fusion partners (e.g., murine myeloma cells) and fusion procedures are also known. One well-known method that may be used for making human CD137 antibodies provided by the present disclosure involves the use of a XENOMOUSE™ animal system. XENOMOUSE™ mice are engineered mouse strains that comprise large fragments of human immunoglobulin heavy chain and light chain loci and are deficient in mouse antibody production. See, e.g., Green et al., Nature Genetics 7:13-21 (1994) and WO2003/040170. The animal is immunized with a CD137 antigen. The CD137 antigen is isolated and/or purified CD137, preferably CD137. It may be a fragment of CD137, such as the extracellular domain of CD137, particularly a CD137 extracellular domain fragment comprising amino acid resides 34-108 or 34-93 of SEQ ID NO: 1. Immunization of animals can be carried out by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane, supra, and U.S. Pat. No. 5,994,619. The CD137 antigen may be administered with an adjuvant to stimulate the immune response. Exemplary adjuvants include complete or incomplete Freund's adjuvant, RIBI (muramyl dipeptides) or ISCOM (immunostimulating complexes). After immunization of an animal with a CD137 antigen, antibody-producing immortalized cell lines are prepared from cells isolated from the immunized animal. After immunization, the animal is sacrificed and lymph node and/or splenic B cells are immortalized. Methods of immortalizing cells include, but are not limited to, transferring them with oncogenes, inflecting them with the oncogenic virus cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Immortalized cells are screened using CD137, a portion thereof, or a cell expressing CD137. CD137 antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art. Antibodies of the disclosure can also be prepared using phage display or yeast display methods. Such display methods for isolating human antibodies are established in the art, such as Achim Knappik, et al., “Fully Synthetic Human Combinatorial Antibody Libraries (HuCAL) Based on Modular Consensus Frameworks and CDRs Randomized with Trinucleotides.” J. Mol. Biol. (2000) 296, 57-86; and Michael J. Feldhaus, et al., “Flow-cytometric isolation of human antibodies from a non-immune Saccharomyces cerevisiae surface display library” Nat Biotechnol (2003) 21:163-170.

In some embodiments, the anti-CD137 antibody is prepared by expressing one or more nucleic acids encoding the anti-CD137 antibody or polypeptide chains thereof in a host cell. In some embodiments, the one or more nucleic acids is a DNA or RNA, and may or may not contain intronic sequences. Typically, the nucleic acid is a cDNA molecule.

Nucleic acids of the disclosure can be obtained using any suitable molecular biology techniques. For antibodies expressed by hybridomas, cDNAs encoding the light and heavy chains of the antibody made by the hybridoma can be obtained by PCR amplification or cDNA cloning techniques. For antibodies obtained from an immunoglobulin gene library (e.g., using phage display techniques), the nucleic acid encoding the antibody can be recovered from the library.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG4 or IgG2 constant region without ADCC effect. The IgG4 constant region sequence can be any of the various alleles or allotypes known to occur among different individuals. These allotypes represent naturally occurring amino acid substitution in the IgG4 constant regions. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat et al (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al., Science 242:423-426 (1988); Huston et al., Proc. Natl. Acad Sci. USA 85:5879-5883 (1988); and McCafferty et al., Nature 348:552-554 (1990)).

In some embodiments, there is provided a vector that comprises one or more nucleic acid molecules encoding an anti-CD137 antibody, described herein. In some embodiments, the vector is an expression vector useful for the expression of the anti-CD137 antibody. In some embodiments, provided herein are vectors, wherein a first vector comprises a polynucleotide sequence encoding a heavy chain variable region as described herein, and a second vector comprises a polynucleotide sequence encoding a light chain variable region as described herein. In some embodiments, a single vector comprises polynucleotides encoding a heavy chain variable region as described herein and a light chain variable region as described herein.

To express an anti-CD137 antibody described herein, DNAs encoding partial or full-length light and heavy chains are inserted into expression vectors such that the DNA molecules are operatively linked to transcriptional and translational control sequences. In this context, the term “operatively linked” means that an antibody gene is ligated into a vector such that transcriptional and translational control sequences within the vector serve their intended function of regulating the transcription and translation of the DNA molecule. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by any suitable methods (e.g., ligation of complementary restriction sites on the antibody gene fragment and vector, or homologous recombination-based DNA ligation). The light and heavy chain variable regions of the antibodies described herein can be used to create full-length antibody genes of any antibody isotype and subclass by inserting them into expression vectors already encoding heavy chain constant and light chain constant regions of the desired isotype and subclass such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the expression vectors of the disclosure typically carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SR promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by any suitable techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies of the disclosure in either prokaryotic or eukaryotic host cells, expression of antibodies in eukaryotic cells, and typically mammalian host cells, is most typical.

In some embodiments, there is provided a host cell containing a nucleic acid molecule provided by the present disclosure. The host cell can be virtually any cell for which expression vectors are available. It may be, for example, a higher eukaryotic host cell, such as a mammalian cell, a lower eukaryotic host cell, such as a yeast cell, and may be a prokaryotic cell, such as a bacterial cell. Introduction of the recombinant nucleic acid construct into the host cell can be effected by calcium phosphate transfection, DEAE, dextran mediated transfection, electroporation or phage infection.

Suitable prokaryotic hosts for transformation include E. coli, Bacillus subtilis, Salmonella typhimurium and various species within the genera Pseudomonas, Streptomyces, and Staphylococcus.

Mammalian host cells for expressing a binding molecule of the disclosure include, for example, Chinese Hamster Ovary (CHO) cells (including dhfr-CHO cells, described in Urlaub and Chasin, Proc. Natl. Acad. Sci. USA 77:4216-4220 (1980), used with a DHFR selectable marker, e.g., as described in Kaufman and Sharp. J. Mol. Biol. 159:601-621 (1982), NS0 myeloma cells, COS cells and Sp2 cells. In particular, for use with NS0 myeloma or CHO cells, another expression system is the GS (glutanine synthetase) gene expression system disclosed in WO 87/04462, WO 89/01036 and EP 338,841. When expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using any suitable protein purification methods.

### IV. Pharmaceutical Compositions, Kits, and Articles of Manufacture

One aspect of the present application provides a composition comprising any one of the anti-CD137 antibodies described herein and/or any one of the CD137-inducing agents (e.g., cytokine, HDAC inhibitor or DNA-damaging agent) described herein. Also provided are pharmaceutical compositions comprising any one of the anti-CD137 antibodies described herein and/or any one of the CD137-inducing agents described herein. In some embodiments, the composition is a pharmaceutical composition comprising: (a) an effective amount of the anti-CD137 antibody; (b) an effective amount of the CD137-inducing agent; and (c) a pharmaceutically acceptable carrier. In some embodiments, the composition further comprises an anti-CD20 antibody (e.g., rituximab). The compositions can be prepared by conventional methods known in the art.

The term “pharmaceutically acceptable carrier” refers to any inactive substance that is suitable for use in a formulation for the delivery of an active agent (e.g., the anti-CD137 antibody and/or the CD137-inducing agent). A carrier may be an antiadherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering an anti-CD137 antibody and/or a CD137-inducing agent is a sterile liquid, such as a solution, suspension, or dispersion, for injection or infusion. Sterile solutions can be prepared by incorporating the antibody in the required amount in an appropriate carrier, followed by sterilization microfiltration. Generally, dispersions are prepared by incorporating the antibody into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of an anti-CD137 antibody and/or a CD137-inducing agent included in the composition will vary depending upon a number of factors, such as the specific anti-CD137 antibody and/or CD137-inducing agent and carriers used, dosage form, and desired release and pharmacodynamic characteristics. The amount of an anti-CD137 antibody and/or a CD137-inducing agent in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In some embodiments, there is provided an article of manufacture comprising materials useful for the treatment of a cancer. The article of manufacture can comprise a container and a label or package insert on or associated with the container. Suitable containers include, for example, bottles, vials, syringes, etc. The containers may be formed from a variety of materials such as glass or plastic. Generally, the container holds a composition, which is effective for treating a cancer, described herein, and may have a sterile access port (for example, the container may be an intravenous solution bag or a vial having a stopper pierceable by a hypodermic injection needle). Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a cancer. The label or package insert may further comprise instructions for administering the composition to a patient.

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a cancer described herein, optionally in combination with the articles of manufacture. Kits of the present application include one or more containers comprising any one of the compositions described herein (or unit dosage form and/or article of manufacture). In some embodiments, the kit further comprises instructions for use in accordance with any of the methods described herein. The kit may further comprise a description of selection of individuals suitable for treatment. Instructions supplied in the kits of the present application are typically written instructions on a label or package insert (e.g., a paper sheet included in the kit), but machine-readable instructions (e.g., instructions carried on a magnetic or optical storage disk) are also acceptable.

For example, in some embodiments, there is provided a kit comprising: (a) a pharmaceutical composition comprising any one of the anti-CD137 antibodies described herein and a pharmaceutically acceptable carrier; (b) a pharmaceutical composition comprising any one of the CD137-inducing agents described herein and a pharmaceutically acceptable carrier; and (c) instructions for administering the pharmaceutical composition to a subject having a cancer. In some embodiments, the kit further comprises a pharmaceutical composition comprising an anti-CD20 antibody (e.g., rituximab).

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The containers may be unit doses, bulk packages (e.g., multi-dose packages) or sub-unit doses. Kits may also include multiple unit doses of the pharmaceutical compositions and instructions for use and packaged in quantities sufficient for storage and use in pharmacies, for example, hospital pharmacies and compounding pharmacies.

### EXAMPLES

The examples below are intended to be purely exemplary of the invention and should therefore not be considered to limit the invention in any way. The following examples and detailed description are offered by way of illustration and not by way of limitation.

### Example 1. CD137 Expression on the Surface of IL-2-Stimulated PBMCs

The following example describes CD137 expression on the surface of peripheral blood mononuclear cells (PBMCs) stimulated with the cytokine IL-2.

Fresh human PBMCs were separated from the whole blood by a density gradient centrifugation method using Ficoll Histopaque. The isolated cells were cultured in 96-well plates (2×10⁵ cells/well) and treated in vitro with different concentrations of IL-2 (0, 100, 1000 IU/mL). After 72 hours, PBMCs were collected and CD137 on the cell surface was stained as follows.

IL-2-stimulated PBMCs were stained with panels of antibodies to detect cell surface markers and thereby identify subsets of cell types in the PBMCs. As shown in Table 1, Panel 1 consisted of anti-human CD45, CD3, CD4, CD8, CD56 and CD137 antibodies, and Panel 2 consisted of anti-human CD45, CD3, CD4, CD20, CD25, CD127 and CD137 antibodies. Subsets of cell types in the PBMCs were sorted by fluorescence-activated cell sorting (FACS). CD137 expression on the different subsets of PBMCs was detected, and the data was analyzed using FlowJo software.

TABLE 1
PBMC FACS Staining Panel

| FACS Staining Panel 1 | | | FACS Staining Panel 2 | | |
|---|---|---|---|---|---|
| T-NK | Markers | Fluorochrome | B-Treg | Markers | Fluorochrome |
| 1 | CD45 | BV510 | 1 | CD45 | BV510 |
| 2 | CD3 | FITC | 2 | CD3 | APC-Cy7 |
| 3 | CD4 | PerCP-Cy5.5 | 3 | CD4 | PerCP-Cy5.5 |
| 4 | CD8 | APC-Cy7 | 4 | CD20 | PE-Cy7 |
| 5 | CD56 | APC | 5 | CD25 | FITC |
| 6 | CD137 | PE-Cy7 | 6 | CD127 | BV421 |
| | | | 7 | CD137 | AF647 |

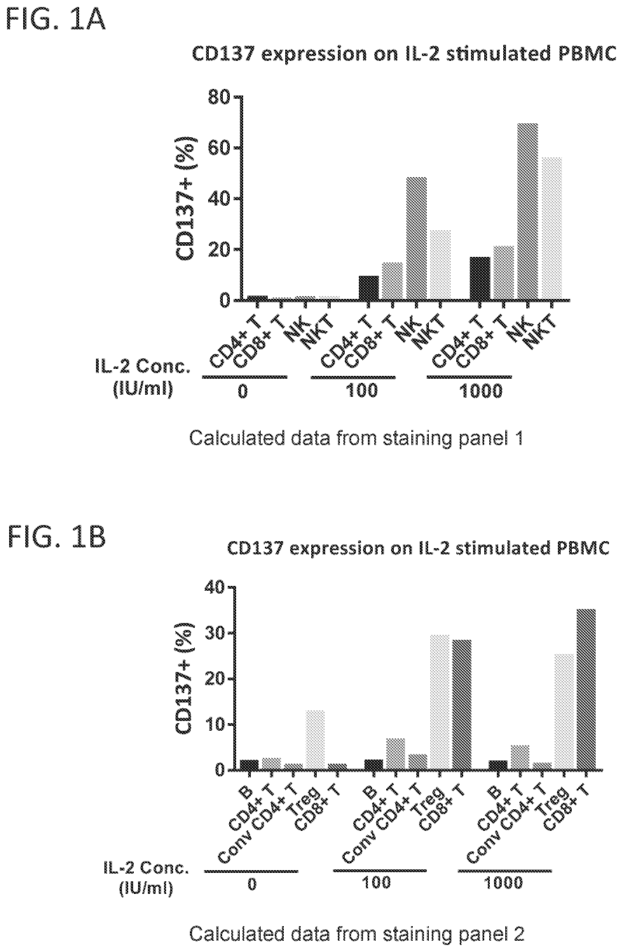

As shown in FIGS. 1A-1B, 100 and 1000 IU/mL of IL-2 strongly induced CD137 expression on human NK, NKT, CD8+ and Treg cells in vitro.

### Example 2. ADG106 in Combination with Continuous High Dose IL-2 in a Mouse Model of Lewis Lung Cancer

The following example describes the therapeutic efficacy of the anti-CD137 antibody ADG106 in combination with IL-2 in a mouse model of Lewis lung cancer.

C57BL/6 mice were transplanted subcutaneously with 5×10⁵ Lewis lung cancer cells. After tumors were established (i.e., having reached a volume of 75 mm³), mice were treated with either vehicle only, ADG106 (5 mg/kg, twice weekly for 4 doses), IL-2 (1.4×10⁷ IU/m², twice a day for 13 consecutive days), or ADG106 (5 mg/kg, twice weekly for 4 doses) and IL-2 (1.4×10⁷ IU/m², twice a day for 13 consecutive days) by intraperitoneal injection. Tumor growth was monitored twice weekly and reported as tumor volume±SEM over time.

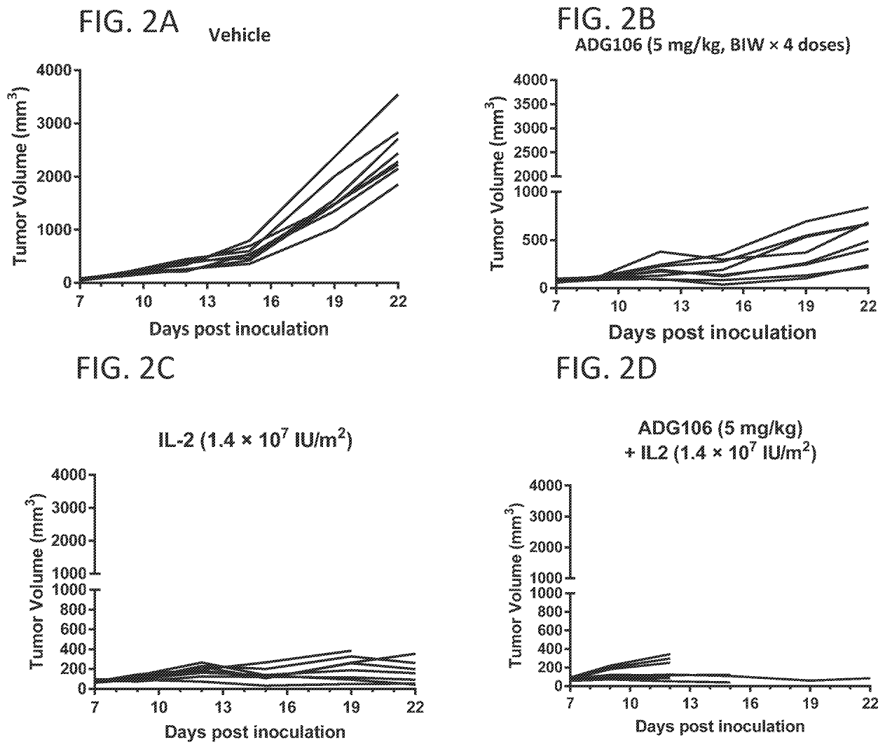

As shown in FIGS. 2A-2D, the continuous high dose IL-2 exhibited robust anti-tumor activity but also caused significant toxicity in mice, resulting in one animal death (⅛) on Day 19 and early termination of all other sick animals (⅞) on Day 22 (FIG. 2C). Combination of ADG106 with the continuous high dose of IL-2 further induced serious toxicity and resulted in accelerated animal death (⅞) from Day 12 to Day 19 (FIG. 2D).

### Example 3. ADG106 in Combination with IL-2 at a High Dose and Low Frequency, or a Continuous Low Dose, in a Mouse Model of Lewis Lung Cancer

This example describes the therapeutic efficacy of anti-CD137 antibody ADG106 in combination with IL-2. IL-2 was administered at high dose in low frequency, or at a continuous low dose, in a mouse model of Lewis lung cancer.

C57BL/6 mice were transplanted subcutaneously with 5×10⁵ Lewis lung cancer cells. After tumors were established (i.e., having reached a volume of 76 mm³), mice were treated with vehicle alone, ADG106 (2.5 mg/kg, twice weekly for 4 doses), IL-2 at a low frequency high dose (1.4-10⁷ IU/m², twice a day, every 3 days for 4 doses), IL-2 at a continuous low dose (2.8×10⁶ IU/m², twice a day for 5 consecutive days), a combination of ADG106 and IL-2 at a low frequency high dose, or a combination of ADG106 and IL-2 at a continuous low dose, by intraperitoneal injection. Tumor growth was monitored twice weekly and is reported as tumor volume±SEM over time.

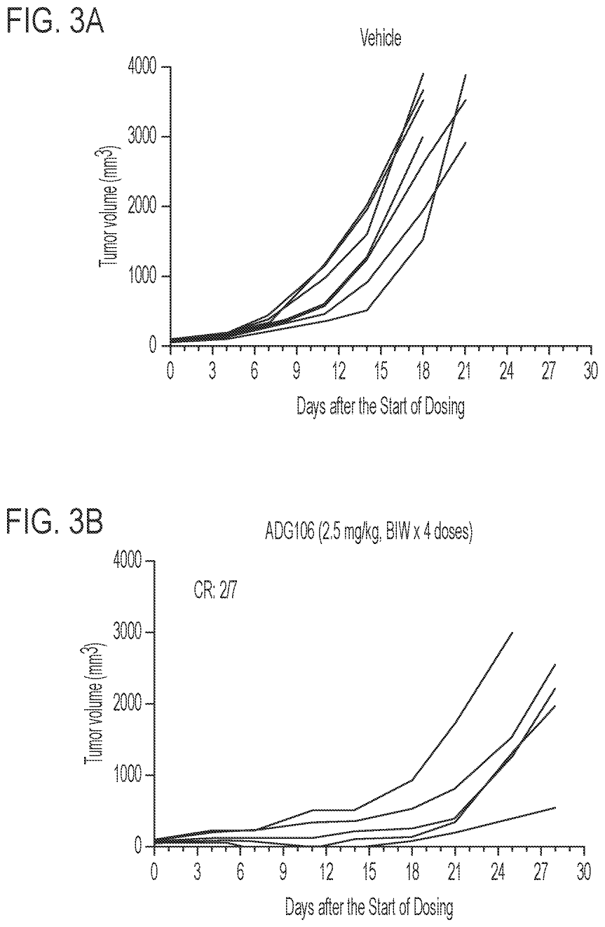

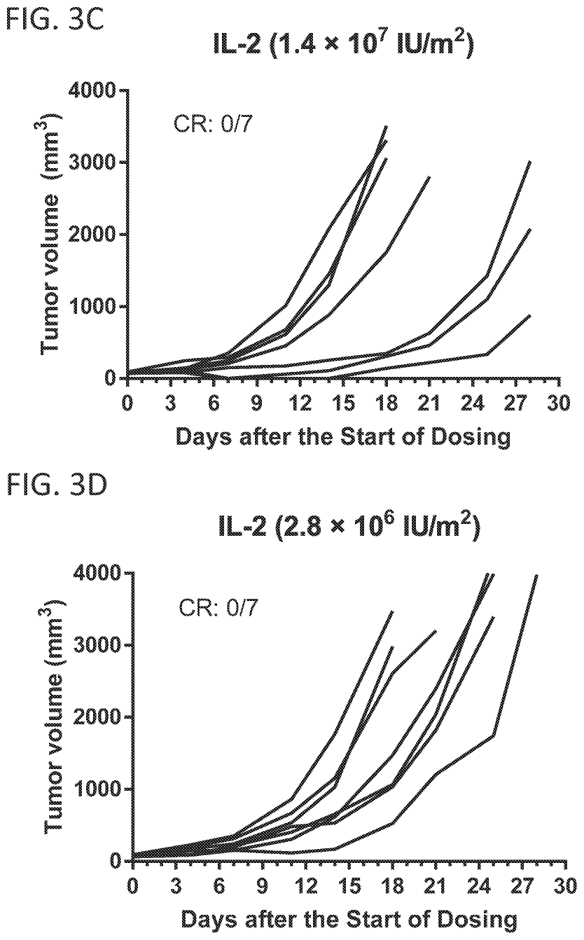

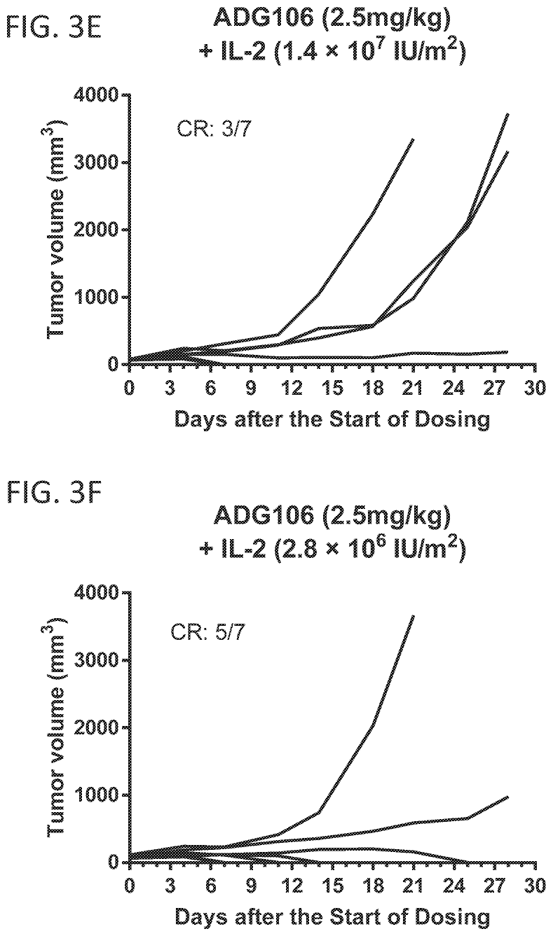

As shown in FIGS. 3A-3F, the low-frequency high dose of IL-2 and the continuous low dose of IL-2 were well tolerated by the mice, but had weak anti-tumor activity (FIGS. 3C-3D). Combination of ADG106 with either IL-2 dose regimen exhibited enhanced anti-tumor efficacy, especially with the continuous low dose IL-2 regimen (FIGS. 3E-3F). No obvious toxicity was observed during the study.

### Example 4. ADG106 in Combination with DNA Damaging Agents in a Mouse Model of A20 B Cell Lymphoma

BALB/c mice (n=8 per group, female, 6-8 weeks old) were transplanted subcutaneously with 5×10⁵ A20 B cell lymphoma cells. After tumors were established (i.e., having reached a volume of 100 mm³), mice were treated with vehicle, ADG106 (5 mg/kg, twice a week for 4 doses by intraperitoneal injection), Bendamustine (12.5 mg/kg, once a day for 4 doses by intraperitoneal injection), or ADG106 (5 mg/kg, twice a week for 4 doses by intraperitoneal injection) in combination with Bendamustine (12.5 mg/kg, once a day for 4 doses by intraperitoneal injection). Tumor growth was monitored twice weekly and reported as mean tumor volume±SEM over time.

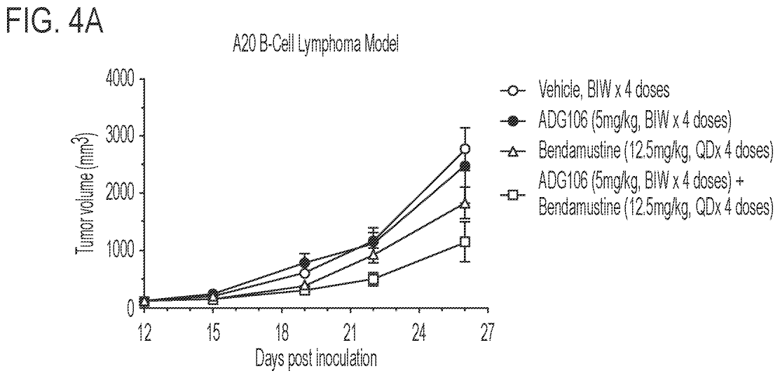

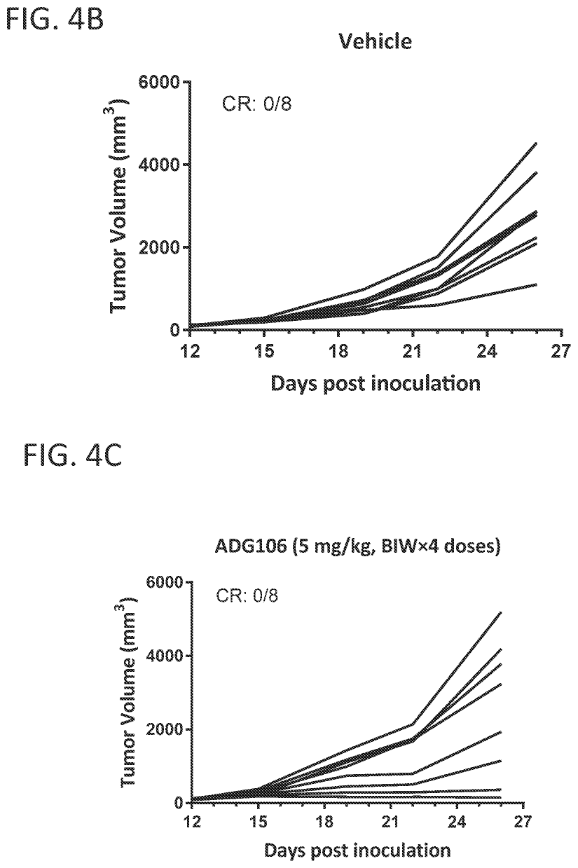

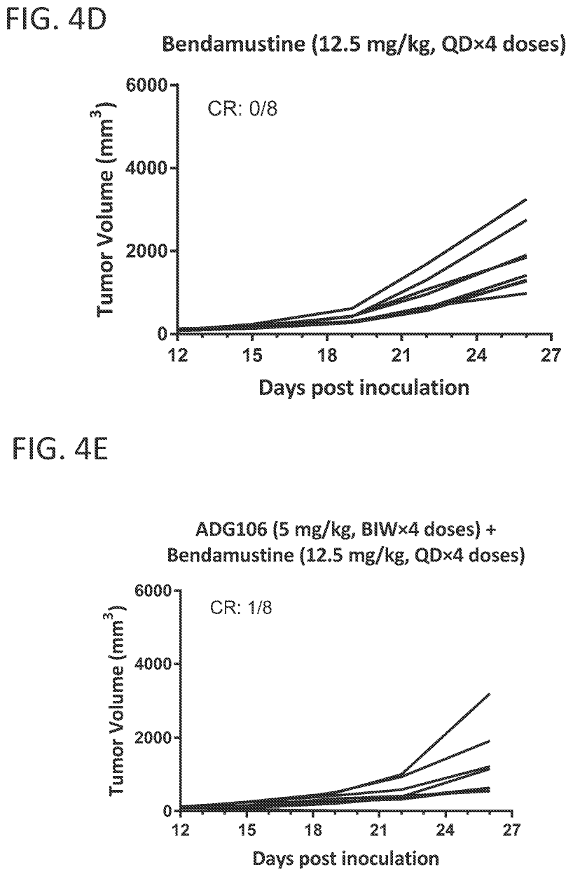

As shown in FIGS. 4A-4E, both ADG106 and Bendamustine treatment were well tolerated by mice as monotherapies. Combination of ADG106 with Bendamustine exhibited enhanced antitumor efficacy compared to ADG106 and Bendamustine monotherapies. Additionally, no obvious toxicity was observed for the combination therapy.

### Example 5. Dose-Dependent Effects of Standard of Care (SOC) Drug Treatments on CD137L Protein Surface Expression

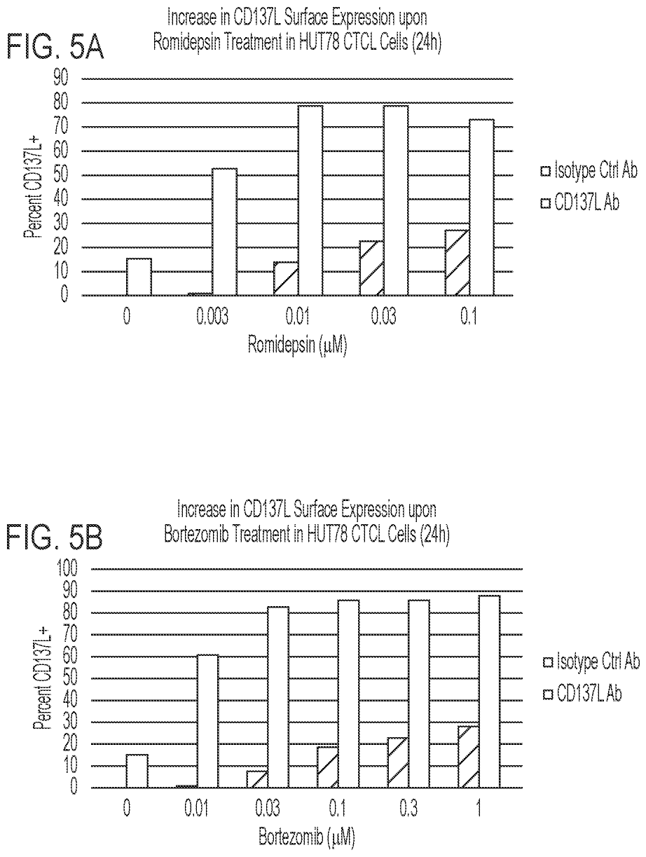

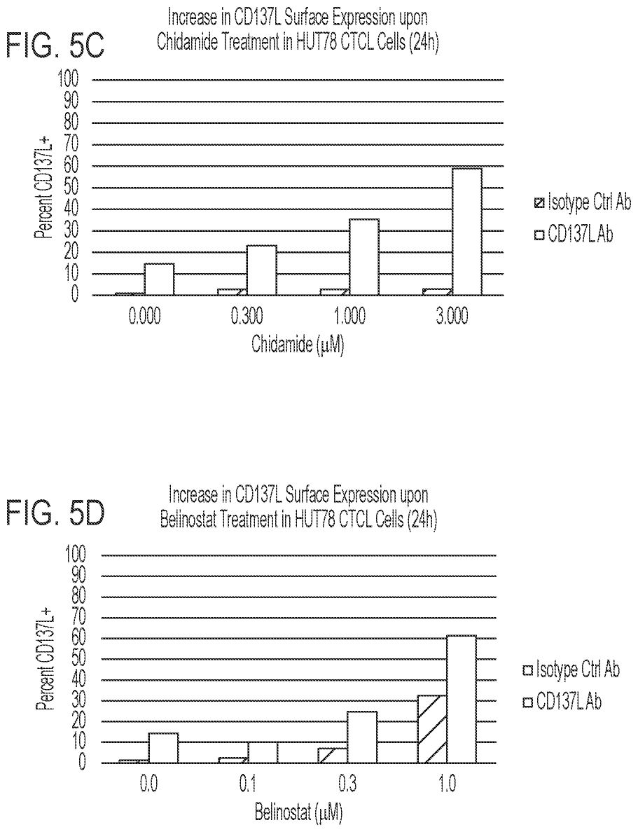

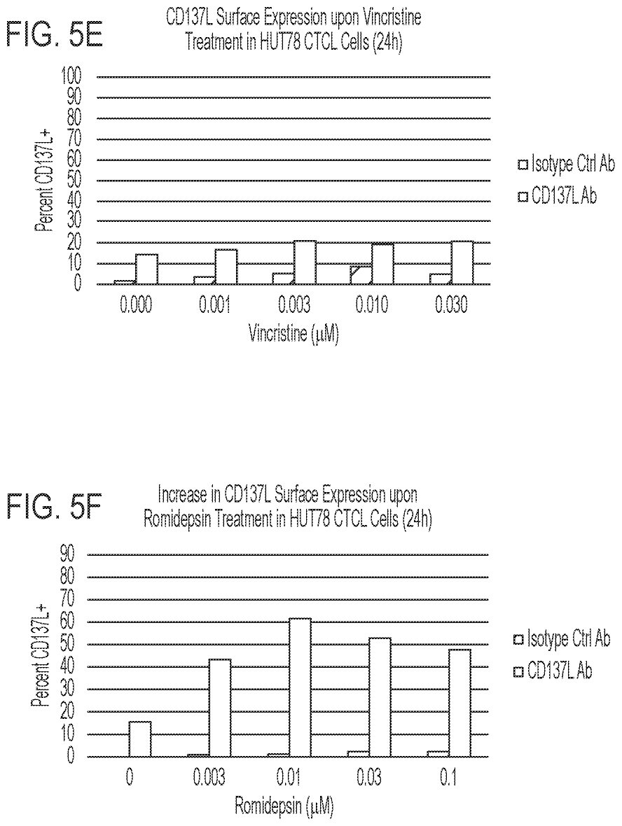

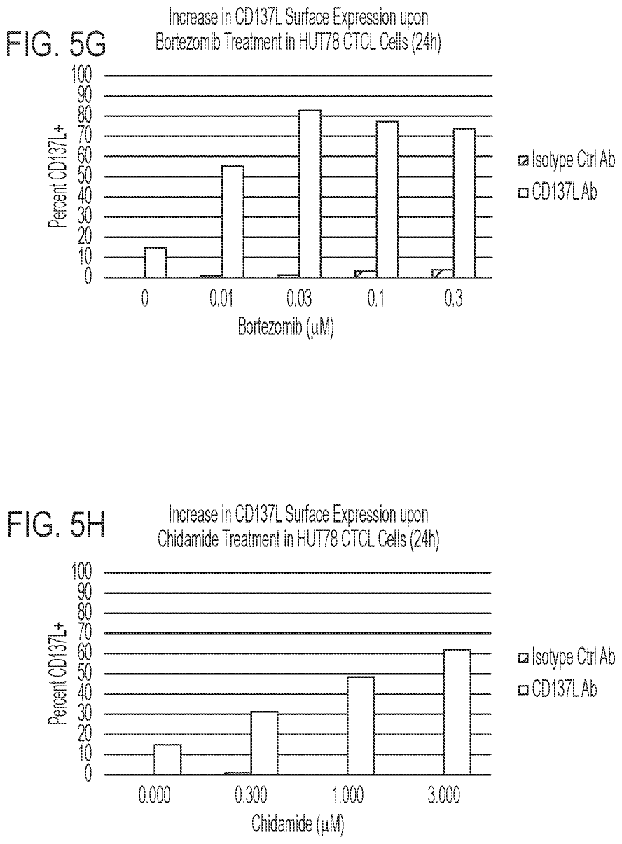

HUT78 cells, cultured in 6-well plates in RPMI-1640 medium containing 20% fetal bovine serum (FBS), were treated with a dose range of Romidepsin (0-0.1 μM), Bortezomib (0-1.0 μM), Belinostat (0-1.0 μM), Chidamide (0-3.0 μM), or Vincristine (0-0.03 μM) for 24 hours. Cells (1×10⁵) were washed twice in Dulbecco's Phosphate Buffered Saline (DPBS), stained with either Phycoerythrin (PE)-conjugated Isotype Control (Biolegend catalog #400112) and anti-human-CD137L (Biolegend catalog #311504) antibodies (1 μL antibody in 100 μL DPBS, FIG. 5A-5E) or PE-Cy7-conjugated Isotype Control (Thermofisher catalog #25-4714-80) and anti-human-CD137L (Thermofisher catalog #25-5906-42) antibodies (1 μL antibody in 100 μL DPBS, FIG. 5F-5H) at 4° C. for 30 minutes, washed twice in DPBS, and resuspended in 100 μL DPBS for flow cytometry analysis.

As shown in FIGS. 5A-5H, upon treatment of Romidepsin (FIGS. 5A and 5F), Bortezomib (FIGS. 5B and 5G), Chidamide (FIGS. 5C and 5H), and Belinostat (FIG. 5D), HUT78 human TCL cells showed dose-dependent upregulation of CD137L protein surface expression. On the other hand, upon treatment of Vincristine (FIG. 5E), HUT78 human TCL cells did not show upregulation of CD137L protein surface expression.

### Example 6. Time-Dependent Effects of SOC Drug Treatments on CD137L Protein Surface Expression

HUT78 cells, cultured in 6-well plates in RPMI-1640 medium containing 20% FBS, were treated with 0.003 μM Romidepsin or 0.01 μM Bortezonib for 2, 6, 16, or 24 hours. Cells (1×103) were washed twice in DPBS, stained with PE-conjugated Isotype Control (Biolegend catalog #400112) and anti-human-CD137L (Biolegend catalog #311504) antibodies (1 μL antibody in 100 μL DPBS) at 4° C. for 30 minutes, washed twice in DPBS, and resuspended in 100 μL DPBS for flow cytometry analysis.

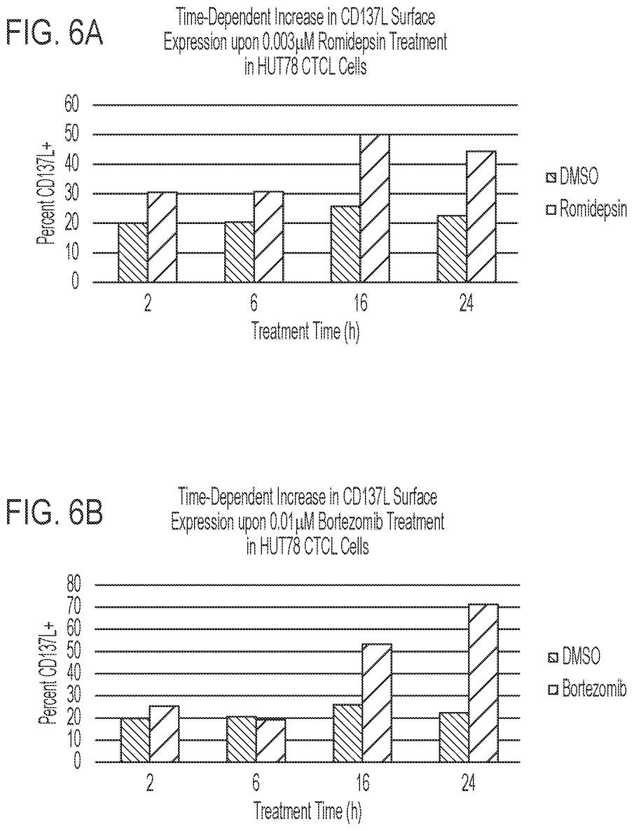

As shown in FIGS. 6A and 6B, upon treatments with Romidepsin (FIG. 6A) and Bortezomib (FIG. 6B), HUT78 human TCL cells showed time-dependent upregulation of CD137L protein surface expression.

### Example 7. Dose-Dependent Effects of SOC Drug Treatments on CD137L mRNA Expression

HUT102, HUT78, and SU-DHL1 cells, cultured in 96-well plates, were treated with a dose range of Romidepsin (0-0.1 μM), Bortezomib (0-1.0 μM), Belinostat (0-10.0 μM), or Vincristine (0-0.03 μM) for 24 hours. Cells were lysed with Lysis Buffer and cell lysates were used for the QuantiGene Plex assay (7-gene multiplex (CD80, CD86, CD274, CD137, CD137L, HPRT1, GAPDH))

Table 2 shows basal expression levels of HPRT1, CD86, CD80, CD274 (PD-L1), GAPDH, TNFSF9 (CD137L/4-1BBL), and TNFRSF9 (CD137/4-1BB) in HUT102, HUT78, and SU-DHL1 human TCL cells. Mean Fluorescence Intensity (MFI) levels, which are surrogates for gene expression levels, are shown. HUT78 and SU-DHL1 cells express very low or no mRNA levels of CD86 and CD80. HUT78 cells express very low or no mRNA levels of CD274 and TNFRSF9 (CD137/4-1BB). When MFI levels are low, slight changes may MFI values may cause dramatic changes in fold-change; thus, caution should be used when interpreting fold-changes in these genes (^{#}).

TABLE 2
Mean Fluorescence Intensity (MFI) levels of HPRT1, CD86, CD80,
CD274 (PD-L1), GAPDH, TNFSF9 (CD137L/4-1BBL), and TNFRSF9 (CD137/4-
1BB) in HUT102, HUT78, and SU-DHL1 human TCL cells

| | | | | CD274 | | TNFSF9 | TNFRSF9 |
|---|---|---|---|---|---|---|---|
| | HPRT1 | CD86 | CD80 | (PD-L1) | GAPDH | (CD137L/4-1BBL) | (CD137/4-1BB) |
| HUT102 | 2427 | 4113 | 3575 | 713 | 27138 | 135 | 1661 |
| HUT78 | 9845 | ^{#}5 | ^{#}5 | ^{#}79 | 29538 | 4353 | ^{#}56 |
| SU-DHL1 | 5492 | ^{#}3 | ^{#}1 | 3294 | 29140 | 577 | 150 |

^{#}Low basal expression, caution should be used when interpreting fold-changes in these genes.

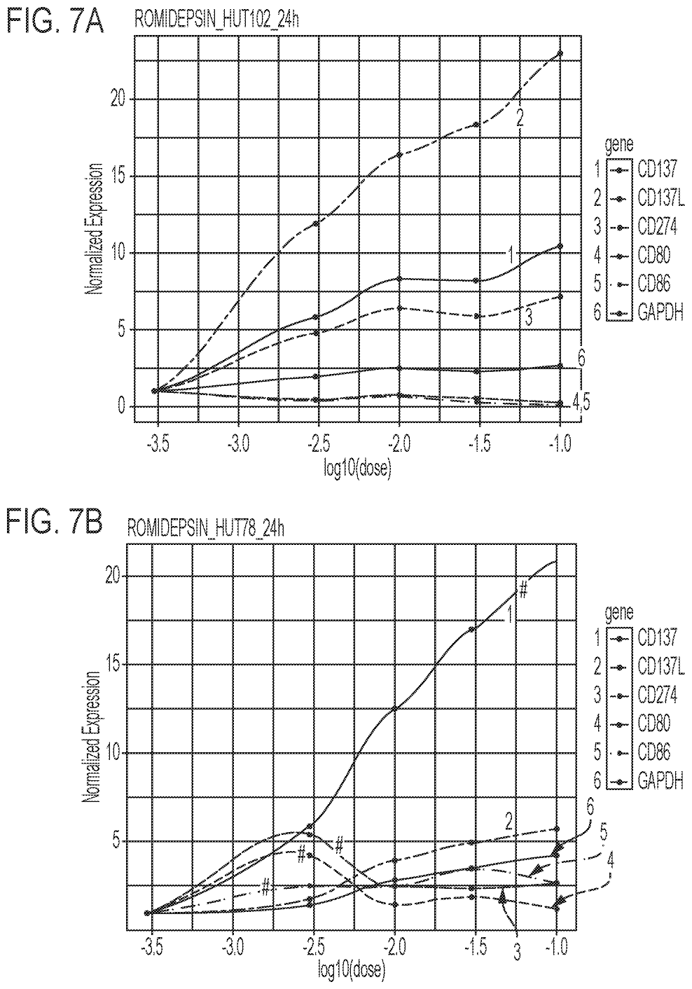

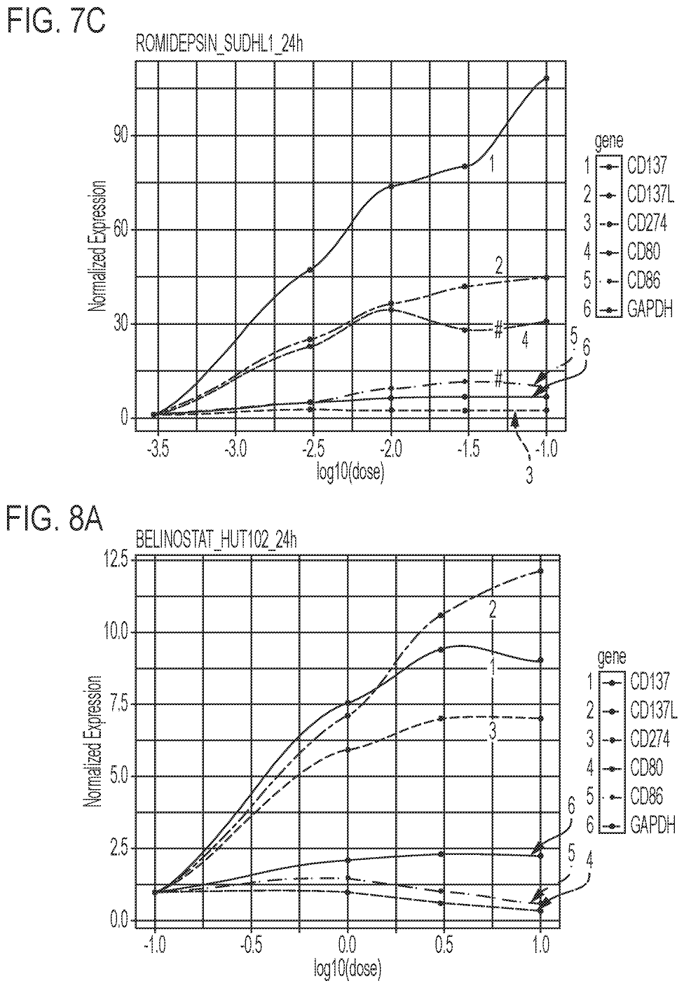

As shown in FIGS. 7A-7C, Romidepsin treatment induced dose-dependent upregulation of CD137L mRNA expression in HUT102 (FIG. 7A), HUT78 (FIG. 7B), and SU-DHL1 (FIG. 7C) human TCL cells. Romidepsin treatment also induced CD137 and CD274 mRNA expression in HUT102 cells (FIG. 7A), and CD137 mRNA expression in SU-DHL1 cells (FIG. 7C).

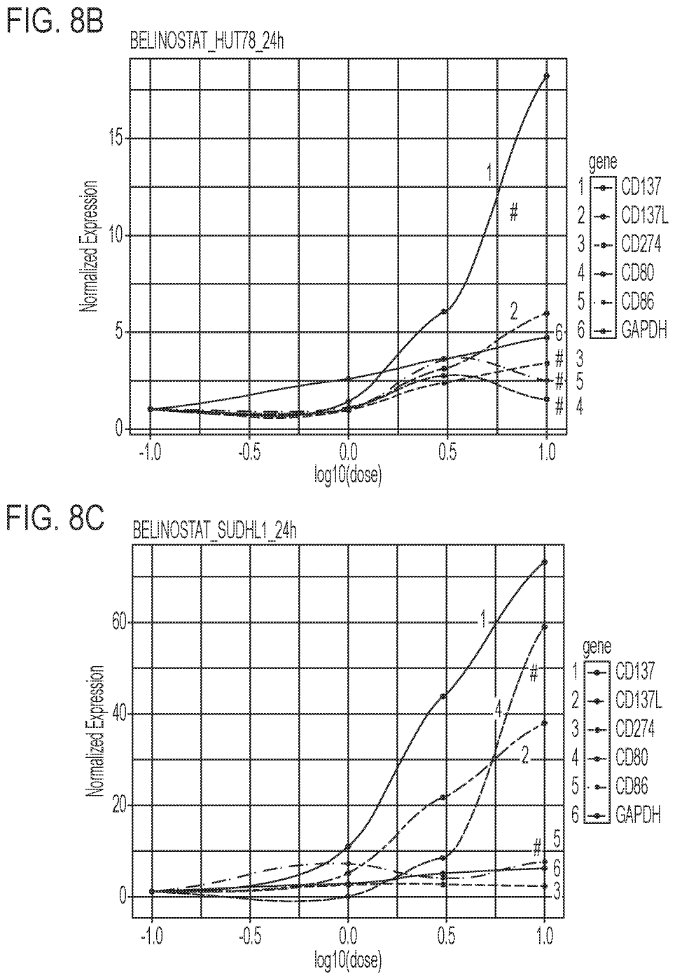

As shown in FIGS. 8A-8C, Belinostat treatment induced dose-dependent upregulation of CD137L mRNA expression in HUT102 (FIG. 8A), HUT78 (FIG. 8B), and SU-DHL1 (FIG. 8C) human TCL cells. Belinostat treatment also induced CD137 and CD274 mRNA expression in HUT102 cells (FIG. 8A), and CD137 mRNA expression in SU-DHL1 cells (FIG. 7C).

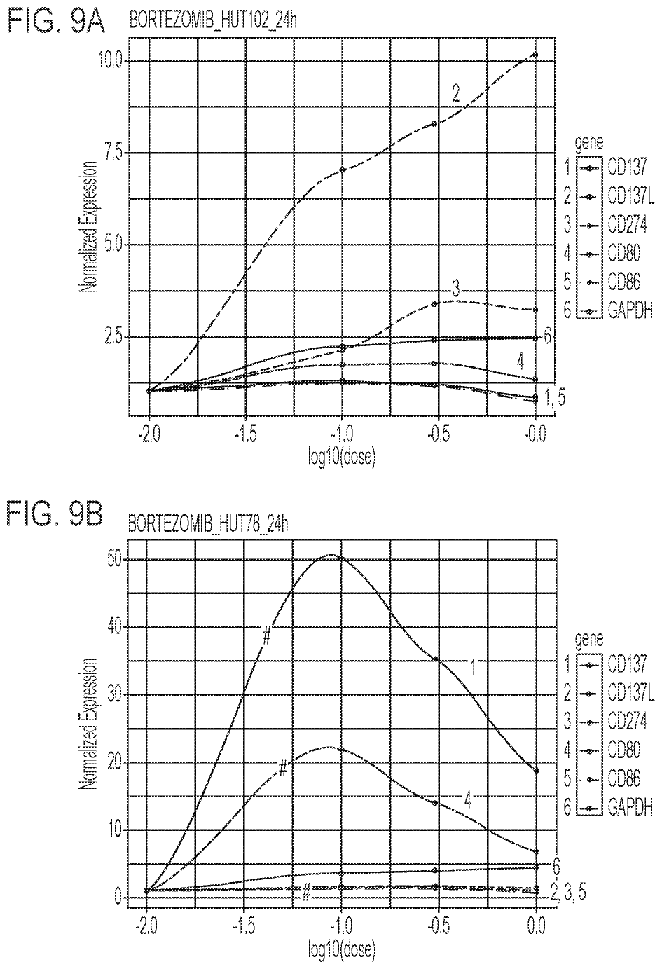

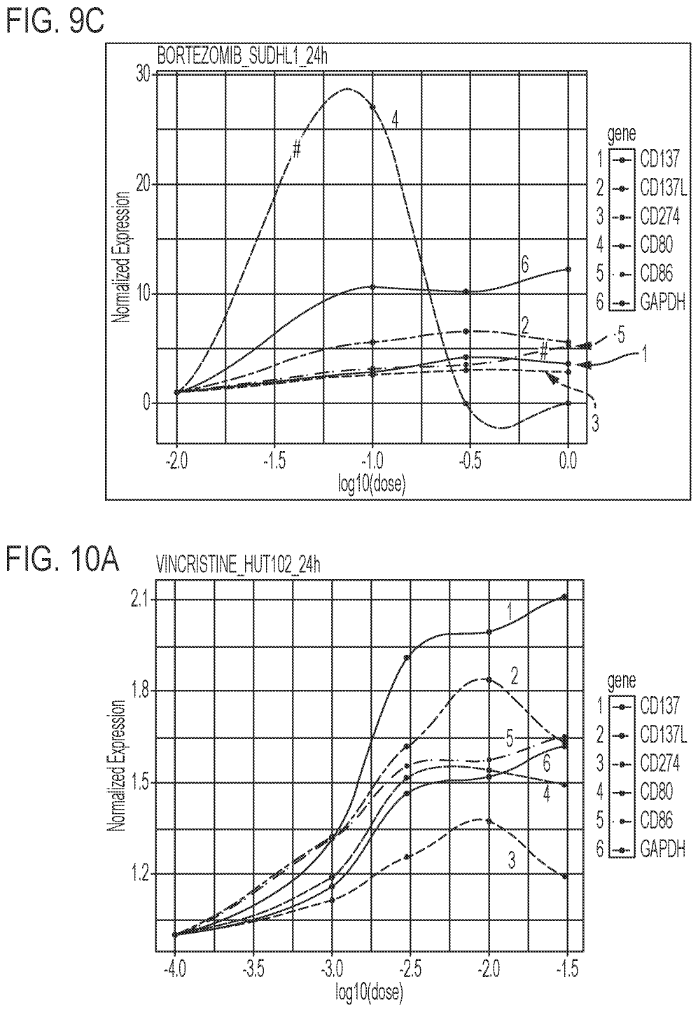

As shown in FIGS. 9A-9C, Bortezomib treatment induced dose-dependent upregulation of CD137L mRNA expression in HUT102 (FIG. 9A) and SU-DHL1 (FIG. 9C), but not HUT78 (FIG. 9B) human TCL cells. Bortezomib treatment also induced CD274 mRNA expression in HUT102 cells (FIG. 9A).

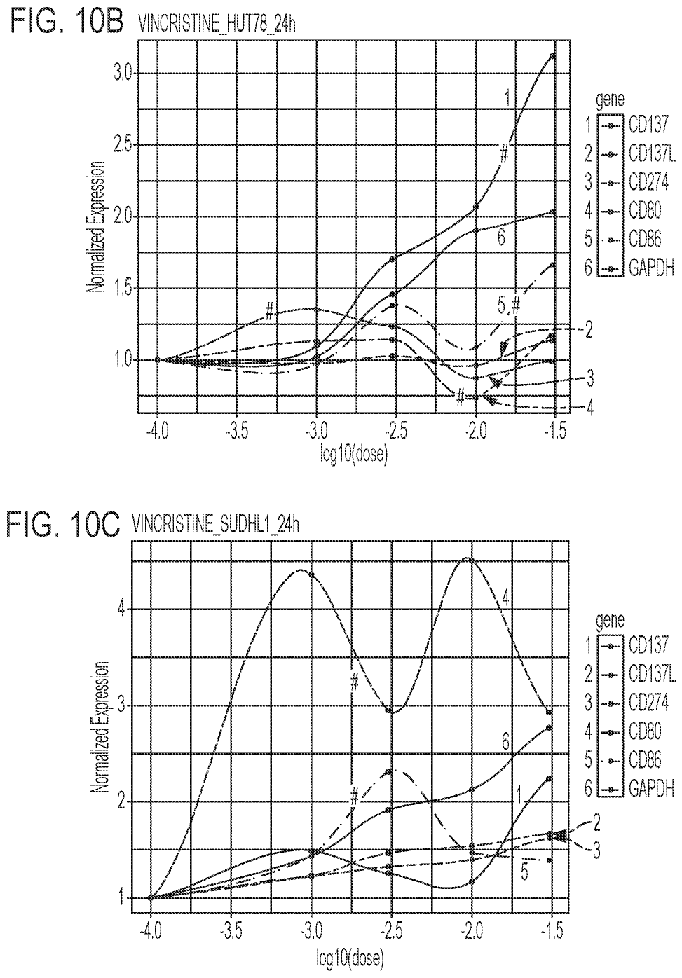

As shown in FIGS. 10A-10C, Vincristine treatment induced upregulation of CD137L mRNA expression in HUT102 (FIG. 10A), but not HUT78 (FIG. 10A B) or SU-DHL1 (FIG. 10A C), human TCL cells. Vincristine treatment also induced CD137 mRNA expression in HUT102 cells (FIG. 10A).

### Example 8. Effects of SOC Drug Treatments on Cell Viability of HUT78 Cells

HUT78 cells, cultured in 96-well plates in RPMI-1640 medium containing 20% fetal bovine serum (FBS), were treated with a dose range of Romidepsin (0-0.1 μM), Bortezomib (0-1.0 μM), or Chidamide (0-3.0 μM) for 24 hours. The CellTiter-Glo assay (Promega) was used to assess cell viability according to the manufacturer's instructions.

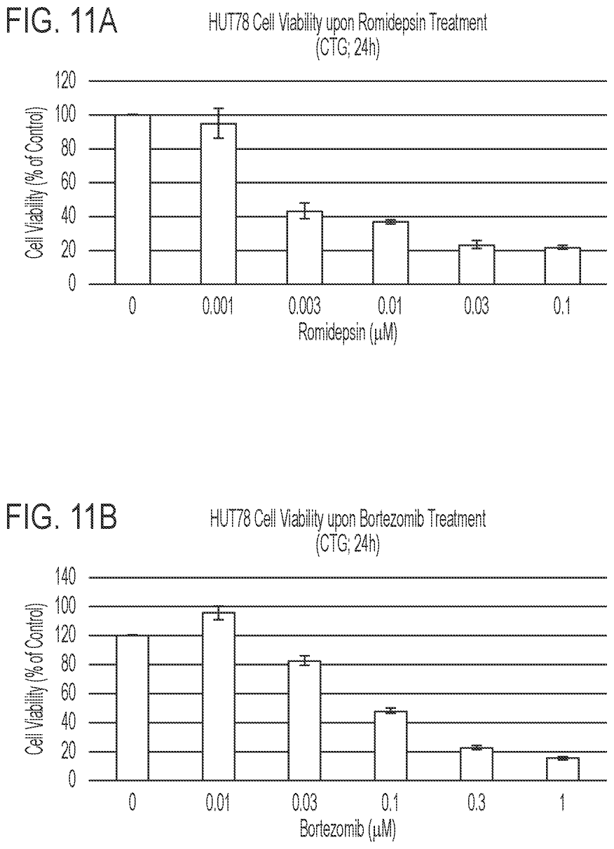

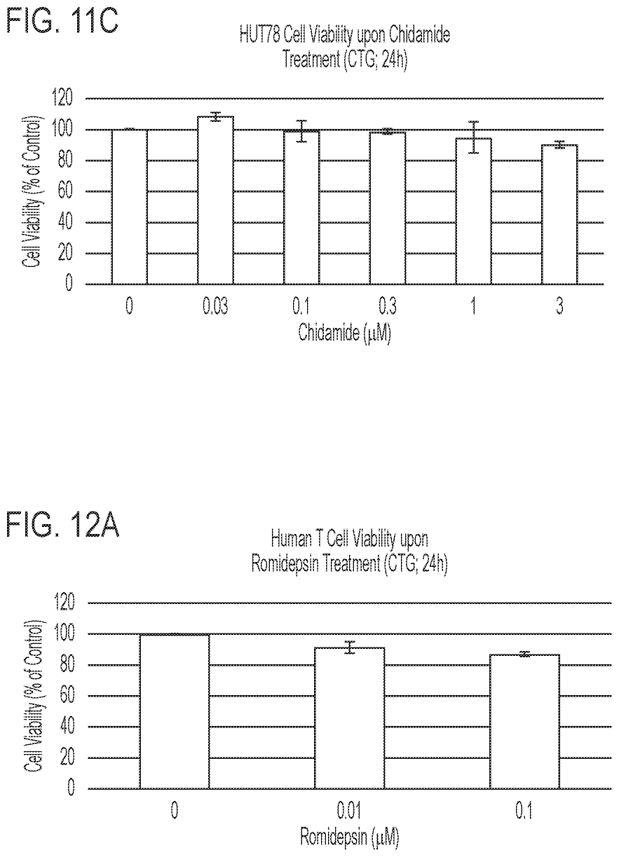

As shown in FIGS. 11A-11C, Romidepsin (FIG. 11A) and Bortezomib (FIG. 11B), but not Chidamide (FIG. 11C), inhibited cell viability of HUT78 human TCL cells in a dose-dependent manner.

### Example 9. Effects of SOC Drug Treatments on Cell Viability of Purified Human T Cells

T cells were purified from human PBMCs (>95% purity, data not shown), cultured in 96-well plates in RPMI-1640 medium containing 10% fetal bovine serum (FBS), and treated with a dose range of Romidepsin (0-0.1 μM), Bortezomib (0-1.0 μM), or Chidamide (0-3.0 μM) for 24 hours. The CellTiter-Glo assay (Promega) was used to assess cell viability according to the manufacturer's instructions.

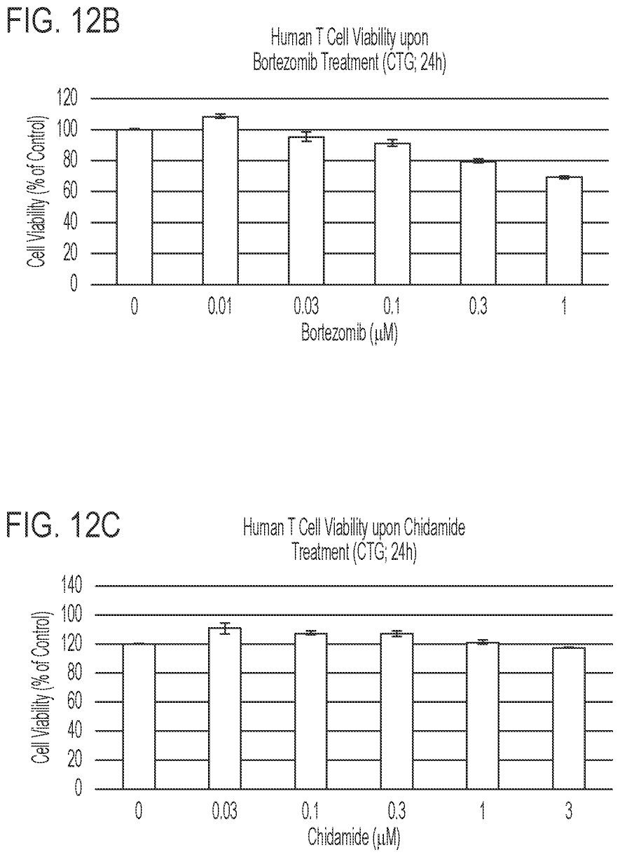

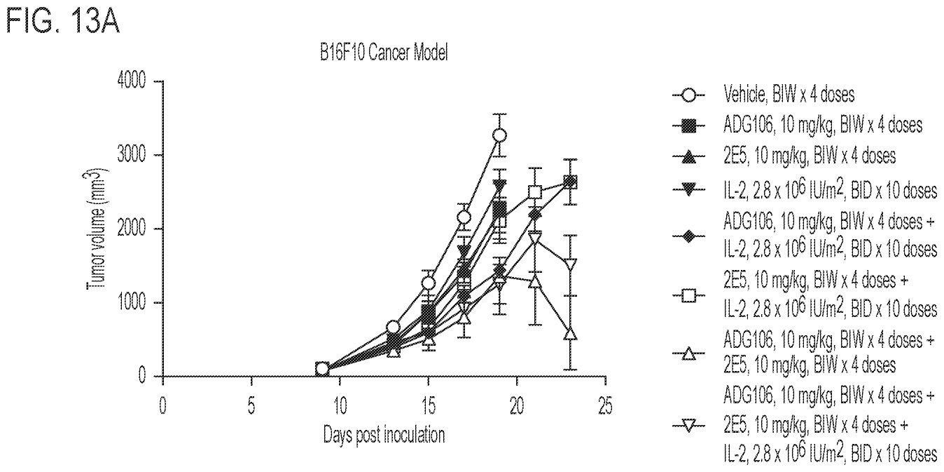

As shown in FIGS. 12A-12C, Romidepsin (FIG. 12A), Bortezomib (FIG. 12B), and Chidamide (FIG. 12C) had minimal or no effect on the viability of purified human T cells.

Table 3, shows a summary for the regulation of CD137L expression by SOC drugs in TCL cell lines. Regulation of CD137L protein surface expression is indicated by the “Protein” columns while regulation of CD137L mRNA expression is indicated by the “mRNA” columns. “Y” indicates the treatment caused upregulation; “N” indicates the treatment did not cause upregulation.

TABLE 3

| | HUT78 | | HUT102 | SU-DHL1 |
|---|---|---|---|---|
| Drugs (type) | Protein | mRNA | mRNA | mRNA |
| Romidepsin (HDACi) | Y | Y | Y | Y |
| Belinostat (HDACi) | Y | Y | Y | Y |
| Chidamide (HDACi) | Y | Not tested | Not tested | |
| Bortezomib | Y | N | Y | Y |
| (Proteasome Inhibitor) | | | | |
| Vincristine | N | N | Y | N |
| (Chemotherapy) | | | | |

### Example 10. ADG106, IL-2, Anti-PD-1 Antibody as Monotherapies or in Combination in B16F10 Mouse Model

C57BL/6 mice (n=8 per group, female. 6-8 weeks old) were transplanted subcutaneously with 5×10⁵ B16F10 murine melanoma cancer cells. After tumors were established (i.e., having reached a volume of 80 mm³), mice were treated with vehicle alone, ADG106 (mouse IgG1, 10 mg/kg, twice weekly for 4 doses), anti-PD1 antibody 2E5 (10 mg/kg, twice weekly for 4 doses), a combination of ADG106 and anti-PD1 antibody 2E5, IL-2 at a continuous low dose (2.8×10⁶ IU/m², twice a day for 5 consecutive days), a combination of ADG106 and IL-2 at a continuous low dose, a combination of anti-PD1 antibody 2E5 and IL-2 at a continuous low dose, or a combination of ADG106, anti-PD1 antibody 2E5 and IL-2 at a continuous low dose by intraperitoneal injection. Tumor growth was monitored twice weekly and is reported as tumor volume±SEM over time.

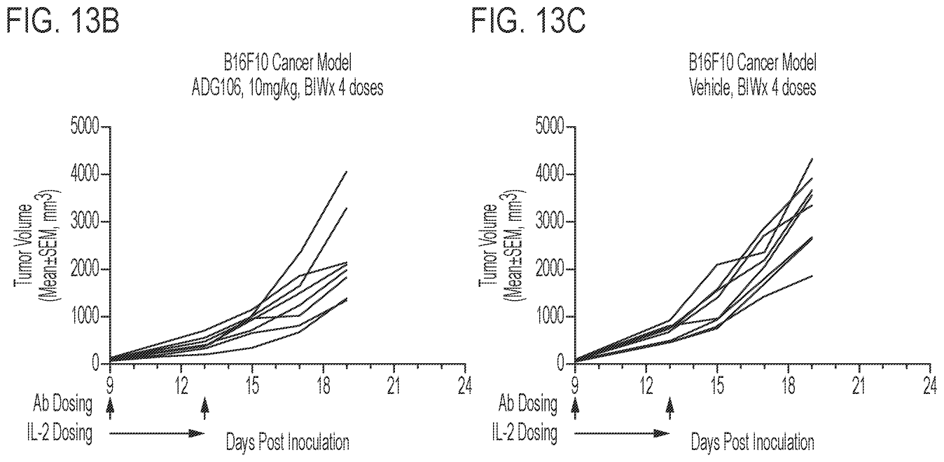

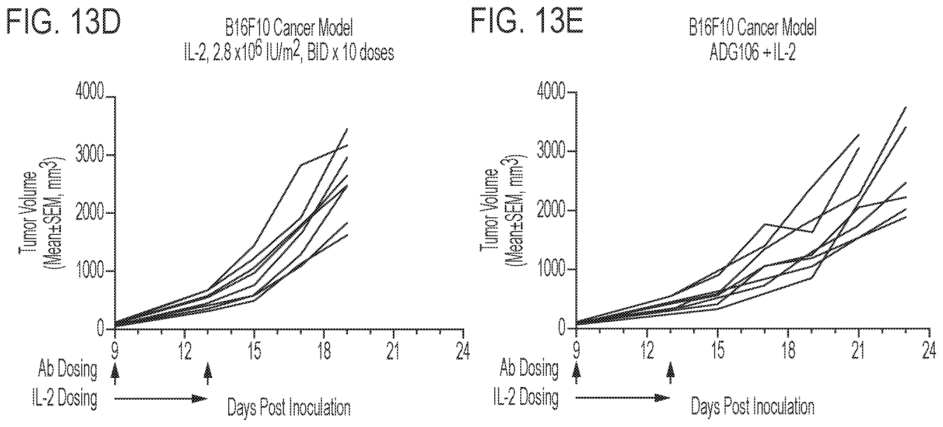

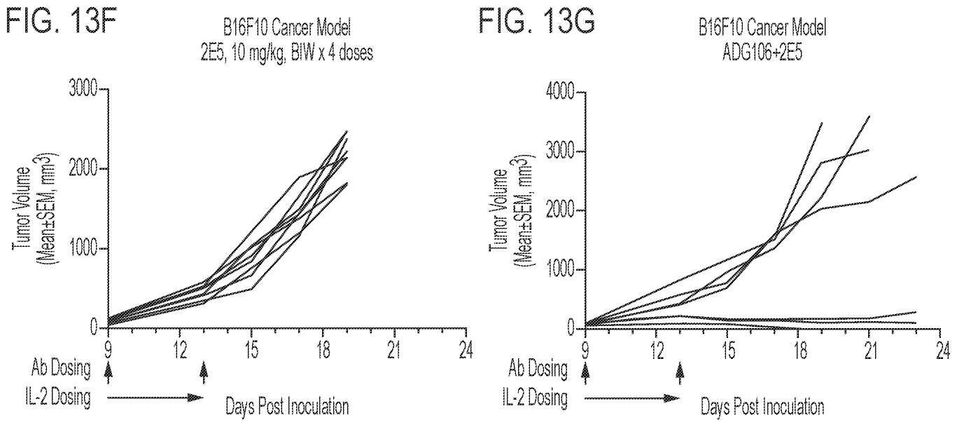

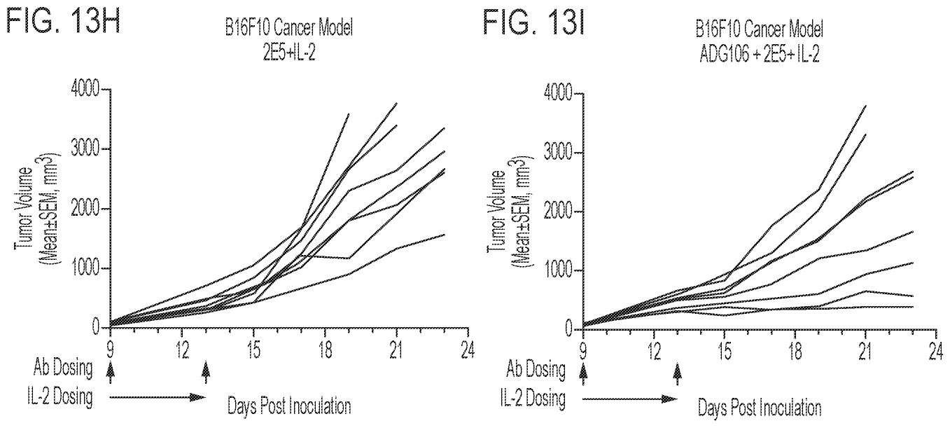

As shown in FIGS. 13A-13I, combination therapy of ADG106, IL-2 and 2E5 exhibited enhanced anti-tumor efficacy compared to ADG106, IL-2 and 2E5 monotherapies.

1. A method of treating a cancer in a subject, comprising administering to the subject: (a) an effective amount of an anti-CD137 antibody that specifically binds to an extracellular domain of human CD137, wherein the antibody binds to one or more amino acid residues selected from the group consisting of amino acid residues 51, 53, 62-73, 83, 89, 92, 95-104 and 112-116 of SEQ ID NO: 1; and (b) an effective amount of an agent that induces expression of CD137 on an immune cell and/or induces expression of CD137L on a cancer cell of the subject.

2. The method of claim 1, wherein the immune cell is selected from the group consisting of CD8+ T cells, regulatory T (Treg) cells, natural killer (NK) cells, and NK-T cells.

3. The method of claim 1, wherein the agent is a cytokine selected from the group consisting of IL-2, IL-12, IL-10 and INFγ.

4-5. (canceled)

6. The method of claim 1, wherein the IL-2 is a wildtype IL-2, a chemically modified IL-2 variant, or an IL-2 analog.

7. The method of claim 1, wherein the IL-2 is aldesleukin or bempegaldesleukin.

8. The method of claim 3, wherein the IL-2 is administered at a dose of no more than about 2.8×10⁶ IU/m².

9-12. (canceled)

13. The method of claim 1, wherein the agent is a histone deacetylase (HDAC) inhibitor selected from the group consisting of belinostat, vorinostat, romidepsin, and chidamide.

14-15. (canceled)

16. The method of claim 1, wherein the agent is a DNA-damaging agent selected from the group consisting of mitomycin, bleomycin, doxorubicin and bendamustine.

17-18. (canceled)

19. The method of claim 1, further comprising administering an effective amount of an anti-CD20 antibody or an immune checkpoint inhibitor.

20. The method of claim 19, wherein the anti-CD20 antibody is rituximab and/or the immune checkpoint inhibitor is an anti-PD-1 antibody.

21-25. (canceled)

26. The method of claim 1, wherein the cancer is a liquid cancer.

27. The method of claim 26, wherein the cancer is non-Hodgkin's lymphoma, T-cell lymphoma, B-cell lymphoma, or multiple myeloma.

28-30. (canceled)

31. The method of claim 1, wherein the cancer is a solid cancer.

32. The method of claim 31, wherein the cancer is selected from the group consisting of breast cancer, ovarian cancer, colorectal cancer, gastric cancer, melanoma, liver cancer, lung cancer, thyroid cancer, kidney cancer, brain cancer, cervical cancer, bladder cancer, and esophageal cancer.

33-34. (canceled)

35. The method of claim 1, wherein the cancer is in adjuvant setting or neoadjuvant setting.

36. The method of claim 1, wherein the anti-CD137 antibody is administered at a dose of no more than 500 mg, no more than about 10 mg/kg, or both.

37-43. (canceled)

44. The method of claim 1, wherein the anti-CD137 antibody is cross-reactive with a CD137 polypeptide from at least one non-human species selected from the group consisting of cynomolgus monkey, mouse, rat and dog.

45. The method of claim 1, wherein the anti-CD137 antibody binds to amino acid residues 51, 63-67, 69-73, 83, 89, 92, 98-104 and 112-114 of SEQ ID NO: 1.

46. The method of claim 1, wherein the anti-CD137 antibody comprises a heavy chain variable region (VH) and a light chain variable region (VL), wherein the VH comprises a HVR-H1 comprising the amino acid sequence of SEQ ID NO: 2, a HVR-H2 comprising the amino acid sequence of SEQ ID NO: 3, and a HVR-H3 comprising the amino acid sequence of SEQ ID NO: 4; and wherein the VL comprises a HVR-L1 comprising the amino acid sequence of SEQ ID NO: 5, a HVR-L2 comprising the amino acid sequence of SEQ ID NO: 6, and a HVR-L3 comprising the amino acid sequence of SEQ ID NO: 7.

47. The method of claim 46, wherein the VH comprises the amino acid sequence of SEQ ID NO: 8, and/or the VL comprises the amino acid sequence of SEQ ID NO: 9.

48. The method of claim 47, wherein the antibody comprises a heavy chain and a light chain, and wherein the heavy chain comprises the amino acid sequence of SEQ ID NO: 10, and/or the light chain comprises the amino acid sequence of SEQ ID NO: 11.

49. The method of claim 1, wherein the anti-CD137 antibody comprises a VH and a VL, wherein the VH comprises a HVR-H1 comprising the amino acid sequence of SEQ ID NO: 12, a HVR-H2 comprising the amino acid sequence of SEQ ID NO: 13, and a HVR-H3 comprising the amino acid sequence of SEQ ID NO: 14; and wherein the VL comprises a HVR-L1 comprising the amino acid sequence of SEQ ID NO: 15, a HVR-L2 comprising the amino acid sequence of SEQ ID NO: 16, and a HVR-L3 comprising the amino acid sequence of SEQ ID NO: 17.

50. The method of claim 49, wherein the VH comprises the amino acid sequence of SEQ ID NO: 18, and/or the VL comprises the amino acid sequence of SEQ ID NO: 19.

51. The method of claim 50, wherein the antibody comprises a heavy chain and a light chain, wherein the heavy chain comprises the amino acid sequence of SEQ ID NO: 20, and/or the light chain comprises the amino acid sequence of SEQ ID NO: 21.

52. The method of claim 1, wherein the anti-CD137 antibody comprises a VH and a VL, wherein the VH comprises a HVR-H1 comprising the amino acid sequence of SEQ ID NO: 22, a HVR-H2 comprising the amino acid sequence of SEQ ID NO: 23, and a HVR-H3 comprising the amino acid sequence of SEQ ID NO: 24; and wherein the VL comprises a HVR-L1 comprising the amino acid sequence of SEQ ID NO: 25, a HVR-L2 comprising the amino acid sequence of SEQ ID NO: 26, and a HVR-L3 comprising the amino acid sequence of SEQ ID NO: 27.

53. The method of claim 52, wherein the VH comprises the amino acid sequence of SEQ ID NO: 28, and/or the VL comprises the amino acid sequence of SEQ ID NO: 29.

54. The method of claim 53, wherein the antibody comprises a heavy chain and a light chain, wherein the heavy chain comprises the amino acid sequence of SEQ ID NO: 30, and/or the light chain comprises the amino acid sequence of SEQ ID NO: 31.

55. The method of claim 1, wherein the anti-CD137 antibody comprises a human IgG1 Fc region or a human IgG4 Fc region.

56-58. (canceled)
